(19) United States
(12) Patent Application Publication
(10) Pub. No.: US 2025/0376687 A1
(43) Pub. Date: Dec. 11, 2025

(54) COMPOSITIONS AND METHODS TO TREAT NEUROLOGICAL DISEASES

(71) Applicant: The Regents of the University of California, Oakland, CA (US)

(72) Inventors: Alysson Renato Muotri, San Diego, CA (US); Lauro Thiago Turaca, San Diego, CA (US); Angels Almenar, La Jolla, CA (US)

(21) Appl. No.: 18/877,063

(22) PCT Filed: Aug. 4, 2023
(86) PCT No.: PCT/US2023/071686

§ 371 Date: Dec. 19, 2024

Related U.S. Application Data
(60) Provisional application No. 63/395,696, filed on Aug. 5, 2022.

(51) Int. Cl. C12N 15/113

(52) U.S. Cl. CPC C12N 15/1136; C12N 2310/113

(57) ABSTRACT

Disclosed is a method of treating a subject who has a neurological disease. The neurological disease may be associated with altered FOXG1 expression. In one aspect, the method includes a step of administering an effective dose of a FOXG1-AS antisense or inhibitory nucleic acid to a subject in need thereof, thereby rescuing the defects associated with altered FOXG1 expression.

### CROSS REFERENCE TO RELATED APPLICATION(S)

This application claims priority to U.S. Provisional Application No. 63/395,696, filed on Aug. 5, 2022, the disclosures of which are incorporated herein by reference in their entirety.

### TECHNICAL FIELD

The present disclosure is directed to methods to prevent and/or treat neurological diseases and neurodevelopmental disorders. Compositions useful in the herein described methods include antisense oligonucleotides (ASOs).

### INCORPORATION BY REFERENCE OF SEQUENCE LISTING

Accompanying this filing is a Sequence Listing entitled, “00015-416WO1.xml” created on Aug. 4, 2023 and having 190,776 bytes of data, machine formatted on IBM-PC, MS-Windows operating system. The sequence listing is hereby incorporated by reference in its entirety for all purposes.

### BACKGROUND

Autism spectrum disorder (ASD) is a complex neurodevelopmental disorder in which different combinations of genetic mutations can contribute to the phenotype. One of these disorders is known as FOXG1 syndrome (FS), which presents variable symptoms such as epilepsy, microcephaly (congenital or postnatal), severe intellectual disability, abnormal or involuntary movements, and unexplained episodes of crying. The syndrome is considered an autosomal dominant condition, which means one copy of the altered gene in each cell is sufficient to cause the disorder. This gene encodes a protein called forkhead box G1 that plays an important role in the development of the embryonic telencephalon. The telencephalon develops into an important region of the brain, which controls most voluntary activity, language, sensory perception, learning, and memory.

FOXG1 syndrome can be caused by mutations in the FOXG1 gene, which prevent the production of the G1 forkhead box or impair the function of the protein. The absence of FOXG1 disrupts normal brain development during embryonic development, inducing premature differentiation and depletion of the progenitor pool.

FoxG1 is an evolutionarily conserved winged helix or forkhead-box (Fox) transcription factor that is expressed in the forebrain. Several studies show that the main role of FoxG1 is to prevent neuronal progenitor cells from undergoing premature differentiation. In mice with a Foxg1 null mutation, the cerebral hemispheres are reduced due to depletion of the progenitor pool due to premature exit from the cycle. cellular and neuronal differentiation, particularly towards layer 1 neurons.

The human FOXG1 gene is located on chromosome 14q12 and contains only 1 coding exon. Four alternative transcripts for FOXG1 (exon 2 to 5) have been identified in the fetal brain. FoxG1 acts as a transcriptional repressor, with several target genes. Among them, cell cycle inhibitors, such as p27Xic in Xenopus 12 and p21Cip in mice.

### SUMMARY

The disclosure provides an oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NOs: 1-14. In one embodiment, the nucleobase sequence of the oligonucleotide is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to any one of SEQ ID NOs: 1-14. In another embodiment, the oligonucleotide consists of a single-stranded modified oligonucleotide. In yet another embodiment of any of the foregoing, the oligonucleotide is complementary to a FOXG1 antisense (FOXG1 AS) molecule (see, e.g., SEQ ID NO:15, 16, 17, 18, or 19) (T can be U or vice-a-versa where appropriate for RNA and DNA). In yet another embodiment of any of the foregoing embodiments, at least one internucleoside linkage is a modified internucleoside linkage. In a further embodiment, at least one modified internucleoside linkage is a phosphorothioate internucleoside linkage. In yet another embodiment, each modified internucleoside linkage is a phosphorothioate internucleoside linkage. In still another embodiment of any of the foregoing embodiments, at least one internucleoside linkage is a phosphodiester internucleoside linkage. In still further embodiments, at least one internucleoside linkage is a phosphorothioate linkage and at least one internucleoside linkage is a phosphodiester linkage. In vet further embodiments of the foregoing, at least one nucleoside comprises a modified nucleobase. In a further embodiment, the modified nucleobase is a 5-methylcytosine. In yet another embodiment of any of the foregoing at least one nucleoside of the modified oligonucleotide comprises a modified sugar. In a further embodiment, the at least one modified sugar is a bicyclic sugar. In still a further embodiment, the bicyclic sugar comprises a 4′-CH(R)-0)-2′ bridge wherein R is, independently, H, C₁₋₁₂ alkyl, or a protecting group. In yet a further embodiment, R is methyl. In another embodiment, R is H. In another embodiment, at least one modified sugar comprises a 2′-O-methoxyethyl group. In still another embodiment of any of the foregoing embodiments, the oligonucleotide comprises a gap segment consisting of 8 to 12 linked deoxynucleosides; a 5′ wing segment consisting of 3 to 5 linked nucleosides; and a 3′ wing segment consisting of 3 to 5 linked nucleosides; wherein the gap segment is positioned between the 5′ wing segment and the 3′ wing segment and wherein a nucleoside of each wing segment comprises a modified sugar. In a further embodiment, each nucleoside of each wing segment comprises a modified sugar. In another embodiment of any of the foregoing, the oligonucleotide consists of 20 linked nucleosides.

The disclosure also provides an antisense oligonucleotide comprising a sequence and/or structure as set forth in Table 1 and 2, wherein the sequence or structure is at least 8-22 nucleotide in length and sequences that are at least 98-99% identical thereto and which inhibit the activity of FOXG1 AS and/or bind to FOXG1 AS.

The disclosure also provides a method of treating a subject having a FOXG1 syndrome, the method including the step of administering to the subject an effective dose of a FOXG1 AS antisense molecule, vector expressing a FOXG1 AS antisense molecule, a FOXG1 AS inhibitory nucleic acid and/or a vector expressing a FOXG1 AS inhibitory nucleic acid. In one embodiment, the FOXG1 AS antisense molecule is an oligonucleotide as described in any of the embodiments herein.

The disclosure also provides a modified oligonucleotide, wherein the modified oligonucleotide is a gapmer consisting of a 5′ wing segment, a central gap segment, and a 3′ wing segment, wherein: the 5′ wing segment consists of 3-5 modified nucleosides, the central gap segment consists of 8-12 nucleosides, and the 3′ wing segment consists of 3-5 modified nucleosides; wherein the modified oligonucleotide has the nucleobase sequence of any one of SEQ ID NOs: 1-14. In one embodiment, the 3′ and/or 5′ wing segments comprise modified nucleobases selected from the group consisting of 2′-OMe, 2′-MOE, LNA, DNA and any combination thereof.

The disclosure provides a single stranded antisense oligonucleotide (ASO) that suppresses the expression and/or activity of a FOXG1 antisense (FOXG1-AS) nucleic acid, wherein the ASO comprises 12 to 50 linked nucleosides. In one embodiment, the ASO has 18 to 20 linked nucleosides. In another embodiment, at least one internucleoside linkage is a modified internucleoside linkage. In still another embodiment, at least one modified internucleoside linkage is a phosphorothioate internucleoside linkage. In a further embodiment, each modified internucleoside linkage is a phosphorothioate internucleoside linkage. In another embodiment, at least one internucleoside linkage is a phosphodiester internucleoside linkage. In a further embodiment, at least one internucleoside linkage is a phosphorothioate linkage and at least one internucleoside linkage is a phosphodiester linkage. In another embodiment, at least one nucleoside comprises a modified nucleobase. In a further embodiment, the modified nucleobase is a 5-methylcytosine. In another embodiment, at least one nucleoside of the ASO comprises a modified sugar moiety. In a further embodiment, at least one modified sugar moiety is a bicyclic sugar moiety. In still a further embodiment, the bicyclic sugar moiety comprises a 4′-CH(R)-0-2′ bridge wherein R is, independently, H, C₁₋₁₂ alkyl, or a protecting group. In still a further embodiment R is methyl or R is H. In another embodiment, the modified sugar moiety comprises a 2′-O-methoxyethyl group. In still another embodiment, the ASO is a gapmer. In a further embodiment, the ASO comprises: a gap segment consisting of 8 to 12 linked deoxynucleosides; a 5′ wing segment consisting of 3 to 5 linked nucleosides; and a 3′ wing segment consisting of 3 to 5 linked nucleosides; wherein the gap segment is positioned between the 5′ wing segment and the 3′ wing segment and wherein a nucleoside of each wing segment comprises a modified sugar moiety. In still a further embodiment, each nucleoside of each wing segment comprises a modified sugar moiety. In a further or another embodiment, the nucleosides making up each wing segment comprises at least two different modified sugar moieties. In a further or another embodiment, the nucleosides making up each wing segment comprises the same modified sugar moiety. In a further or another embodiment, the modified sugar moiety comprises a 2′-O-methoxyethyl group. In another embodiment, the ASO has a nucleobase sequence that comprises at least 15 consecutive nucleobases of any of the nucleobase sequences of SEQ ID NOs: 1-14. In yet another embodiment, the ASO has a nucleobase sequence of any one of SEQ ID NOs:1-14. In still another embodiment, the ASO has the sequence of SEQ ID NO:2. In still another embodiment, the ASO has a nucleobase sequence of any one of SEQ ID NOs:2, 3 or 4. In yet another embodiment, the ASO is a gapmer consisting of a 5′ wing segment, a central gap segment, and a 3′ wing segment, wherein: the 5′ wing segment consists of 3-5 modified nucleosides, the central gap segment consists of 8-12 nucleosides, and the 3′ wing segment consists of 3-5 modified nucleosides; wherein a modified nucleoside of each wing segment comprises a modified sugar moiety; and wherein the ASO has the nucleobase sequence of any one of SEQ ID NOs: 1-14. In a further embodiment, the ASO has a nucleobase sequence of any one of SEQ ID NOs:2, 3 or 4. In another embodiment, each modified nucleoside of each wing segment comprises a modified sugar moiety. In a further embodiment, the modified nucleosides making up each wing segment comprises at least two different modified sugar moieties. In another or further embodiment, the modified nucleosides making up each wing segment comprises the same modified sugar moiety. In still another or further embodiment, the modified sugar moiety comprises a 2′-O-methoxyethyl group. In another embodiment, the FOXG1-AS nucleic acid has a sequence selected from the group consisting of SEQ ID NO:15, 16, 17, 18 and 19 or a sequence that is at least 80% identical to SEQ ID NO:15, 16, 17, 18 or 19. The disclosure further provides a pharmaceutical composition comprising any of the preceding ASO embodiment and a pharmaceutically acceptable carrier, diluent and/or excipient. In a further embodiment, the pharmaceutical composition is formulated for parenteral delivery. In still another embodiment, the pharmaceutical composition is formulated for intracerebroventricular injection.

The disclosure also provide a method of treating a subject having a neurological or neurodegenerative disease in need of treatment thereof, comprising: administering a therapeutically effective amount of the pharmaceutical composition as set forth herein.

The disclosure also provides a method of increasing the expression of a FOXG1 in a cell, comprising contacting the cell with a composition comprising an antisense oligonucleotide (ASO) complementary to a target nucleic acid having a sequence selected from SEQ ID NO:15, 16, 17, 18, and 19 or a sequence that is at least 80% identical to SEQ ID NO:15, 16, 17, 18 or 19. In a further embodiment, the cell is a located in a brain of a subject. In still a further embodiment, the subject is a mammal, and preferably a human. In another embodiment, the subject comprises a mutant FOXG1 gene. In still another embodiment, the subject has FOXG1 syndrome or Alzheimer's disease. In still another or further embodiment, the FOXG1 nucleic acid is a ribonucleic acid (RNA). In another embodiment, the ASO has 18 to 20 linked nucleosides. In still another embodiment, at least one internucleoside linkage of the ASO is a modified internucleoside linkage. In a further embodiment, at least one modified internucleoside linkage is a phosphorothioate internucleoside linkage. In another embodiment, each modified internucleoside linkage is a phosphorothioate internucleoside linkage. In yet another embodiment, at least one internucleoside linkage of the ASO is a phosphodiester internucleoside linkage. In a further embodiment, at least one internucleoside linkage of the ASO is a phosphorothioate linkage and at least one internucleoside linkage of the ASO is a phosphodiester linkage. In still another embodiment, at least one nucleoside of the ASO comprises a modified nucleobase. In a further embodiment, the modified nucleobase is a 5-methylcytosine. In yet another embodiment, at least one nucleoside of the ASO comprises a modified sugar moiety. In a further embodiment, the at least one modified sugar moiety is a bicyclic sugar moiety. In still a further embodiment, the bicyclic sugar moiety comprises a 4′-CH(R)-0-2′ bridge wherein R is, independently, H, C₁₋₁₂ alkyl, or a protecting group. In yet a further embodiment, R is methyl or H. In another embodiment, the modified sugar moiety comprises a 2′-O-methoxyethyl group. In yet another embodiment, the ASO is a gapmer. In a further embodiment, the ASO comprises: a gap segment consisting of 8 to 12 linked deoxynucleosides; a 5′ wing segment consisting of 3 to 5 linked nucleosides; and a 3′ wing segment consisting of 3 to 5 linked nucleosides; wherein the gap segment is positioned between the 5′ wing segment and the 3′ wing segment and wherein a nucleoside of each wing segment comprises a modified sugar moiety. In still a further embodiment, each nucleoside of each wing segment comprises a modified sugar moiety. In another embodiment, the nucleosides making up each wing segment comprises at least two different modified sugar moieties. In yet another embodiment, the nucleosides making up each wing segment comprises the same modified sugar moiety. In still another embodiment, the modified sugar moiety comprises a 2′-O-methoxyethyl group. In yet another embodiment, the ASO has a nucleobase sequence that comprises at least 15 consecutive nucleobases of any of the nucleobase sequences of SEQ ID NOs: 1-14. In still another embodiment, the ASO has a nucleobase sequence of any one of SEQ ID NOs:1-14. In a further embodiment, the ASO has a nucleobase sequence of any one of SEQ ID NOs:2, 3 or 4. In another embodiment, the ASO is a gapmer consisting of a 5′ wing segment, a central gap segment, and a 3′ wing segment, wherein: the 5′ wing segment consists of 3-5 modified nucleosides, the central gap segment consists of 8-12 nucleosides, and the 3′ wing segment consists of 3-5 modified nucleosides; wherein a modified nucleoside of each wing segment comprises a modified sugar moiety; and wherein the ASO has the nucleobase sequence of any one of SEQ ID NOS: 1-14. In a further embodiment, the ASO has a nucleobase sequence of any one of SEQ ID NOs:2, 3 or 4. In still another embodiment, each modified nucleoside of each wing segment comprises a modified sugar moiety. In a further embodiment, the modified nucleosides making up each wing segment comprises at least two different modified sugar moieties. In still another embodiment, the modified nucleosides making up each wing segment comprises the same modified sugar moiety. In another embodiment, the modified sugar moiety comprises a 2′-O-methoxyethyl group.

The disclosure also provides a method of treating or ameliorating a FOXG1 syndrome in a subject having, or at risk of having, the FOXG1 syndrome, comprising administering to the subject an antisense oligonucleotide as set forth herein, wherein the antisense oligonucleotide comprises a sequence complementary to a sequence that is at least 80%, 90%, 92%, 95%, 97%, 98%, 99% or 100% identical to SEQ ID NO:15, 16, 17, 18, or 19.

In a particular embodiment, the disclosure further provides a method of treating a subject having Alzheimer's disease, comprising: administering a therapeutically effective amount of a pharmaceutical composition disclosed herein, or a therapeutically effective amount of an ASO disclosed herein.

The disclosure also provides methods of treating FOXG1 syndrome comprising administering a de-methylating agent so as to increase FOXG1 expression. Examples of de-methylating agents include, but are not limited to, cytidine derivatives, including 5-azacytidine and 5-azadeoxycytidine; and procainamide and derivatives, such as procaine.

The disclosure also provides a method of screening agents for use in treating FOXG1 syndrome, the method comprising generating neuronal organoids from induced pluripotent stem cells generated from a subject having FOXG1 syndrome; contacting the organoid with an agent and determining if the agent increases FOXG1 expression.

The disclosure also provides pharmaceutical compositions comprising the oligonucleotides or modified oligonucleotides of the disclosure and a pharmaceutically acceptable diluent or carrier.

### BRIEF DESCRIPTION OF THE DRAWINGS

FIG. 1 shows Standardization of messenger RNA amplification of the FOXG1 gene by qPCR.

FIG. 2 shows a graph of MALAT1 expression in Wild type organoids 3 months (ANOVA-p<0.05).

FIG. 3 shows graphs of relative expression of FOXG1 antisense (left) and FOXG1 gene (right) in wild-type organoids (3 months) treated with 12 different ASOs (ANOVA-P<0.05).

FIG. 4 shows graphs of relative expression of FOXG1 antisense (left) and FOXG1 gene (right) in wild-type organoids (3 months) with the 3 best candidate ASOs (ANOVA-P<0.05).

FIG. 5 shows epigenetic regulation of FOXG1 in human brain cells. Treatment of neural progenitor cells (NPCs) with epigenetic drugs upregulate the expression of FOXG1.

FIG. 6A-H provides (A)—bright-field microscopy images of wild type and FOXG1 mutant Brain cortical organoids (BCOs) over 2 and 4 weeks of culture in vitro. (B)—BCO size distribution at 2 and 4 weeks in vitro, for 2 patients carrying Q86fx*35 mutation compared with neurotypical. (C)—Relative expression (RT-qPCR) of FOXG1 gene in Neurotypical and FOXG1 mutants BCOs evaluated at 1, 2, 3 and 4 weeks in vitro. N=2 subjects. (D)—Analysis of protein expression by western blotting in two neurotypical clones, two clones carrying the W308X mutation (mutant 1) and two clones carrying the Q86fx*35 mutation (mutant 2). As a positive control, HEK cells overexpressing FOXG1 were used. Normalization was performed using B-actin. (E)—Flow cytometry performed on neurotypical and mutant BCOs. The FOXG1 mouse antibody (abcam cat. ab 196868) was used in analyze of a minimum of 10 k events. (F)—Graph of FOXG1 positive cells present in neurotypical and mutant BCOs detected by flow cytometry. (G)—Immunofluorescence of organoids for detection of FOXG1, Nestin, MAP2, CC3 and Ki67 markers. Fluorescence images after immunostaining FOXG1 (red), Nestin (green-left), MAP2 (green-right), CC3 (purple) and Ki67 (yellow) in neurotypical and FOXG1 organoids at 4 weeks in vitro. (H)—Fluorescence quantification for analyzed markers in 4-week BCOs.

FIG. 7A-B provides (A) analysis of markers for cell cluster identification through scRNAseq. Populations were defined using already established markers such as MAPT for mature neurons, DCX for postmitotic neurons, TTYH1 for radial glia, TOP2A for proliferative cells, PDE1A for glutamatergic neuroblasts, SLIT2 for glioblasts, and SOX10 for Oligodendrocyte progenitor cells (OCPs). On the right, it showed the identification of FOXG1 expression after at 1, 3, 6 and 10 months of neurotypical BCOs. (B)—Analysis of scRNAseq of mutant and control BCOs at 4 months. Shown on the right are some subpopulation markers and their UMAP2×UMAP1 distribution such as VIM and MKI67 for dividing Radioglia (RG), GADD45G for Oligodendrocyte/NPCDCX, TTYH1 and MAPT for RG, NEUROD2 for glutamatergic cells, and OLIG1 for intermediate Oligodendrocyte progenitor cells (OPCs). The scRNA-Seq data show lower percentages of cells in N-GABA and higher N-Glut neuronal subpopulations in mutants BCOs compared to control. The data shows Oligodendrocyte progenitor cell (OPC) were in less proportion in mutants. Radial glial progenitor cells (RGPs) cells also were found in smaller amounts in W308X CtOs.

FIG. 8A-H shows (A)—Tests of 14 ASOs in BCOs with two doses of 5 uM for 4 days. FOXG1 RNA expression using the TAQMAN system (RT-qPCR). (B)—Verification of FOXG1-AS silencing through expression of messenger RNA using the SYBER system (RT-qPCR). (C) and (D)—Analysis of the RNA expression of FOXG1-As and FOXG1, respectively, after co-treatment with ASO4 and sodium butyrate. (E) and (F)—Verification of monoallelic expression in BCO carrying the W308X and G224S mutation, respectively. After treatment with ASO4 it was possible to verify the reactivation of the inactive allele. (G)—Immunofluorescence in reporter cell FOXG1-NEON after treatment with ASO4. All treatments were performed with 5 uM for 4 days. (H)—Quantification of total fluorescence obtained through expression of NEON in reporter line after treatment.

FIG. 9 shows gene regulatory network model of cranial neural crest cell (CNCC) development (p-adj 0.002) altered in Q68fs*35 VS Control (Neurotypical). Most interactions in the model are proposed to regulate the transcription of core factors involved in neural crest and downstream progenitor specification.

FIG. 10 shows differential expression analysis of the W308X vs Control+Rescue lines revealed alterations in genes involved in active pathways in Fetal brain (p-adj 0.002).

FIG. 11 shows differential expression analysis of the W308X vs Control+Rescue lines revealed altered genes that are involved in the Pathway MECP2 and Associated Rett Syndrome (p-adj-0.05541).

FIG. 12A-E shows (A)—Scheme of the FOXG1-AS gene region and the target regions of ASOs. (B and C)—Processed and unprocessed FOXG1-AS expression, qualitative analysis in agarose gel, and quantitative by RT-qPCR (Syber), respectively. (D)—Tests of ASO MALAT1 in different concentrations (0.1 uM, 1 um, and 5 uM) in 2-month organoids versus decrease in MALAT1 gene expression. (E)—Test of use of lipofectamine and efficiency in decreasing gene expression.

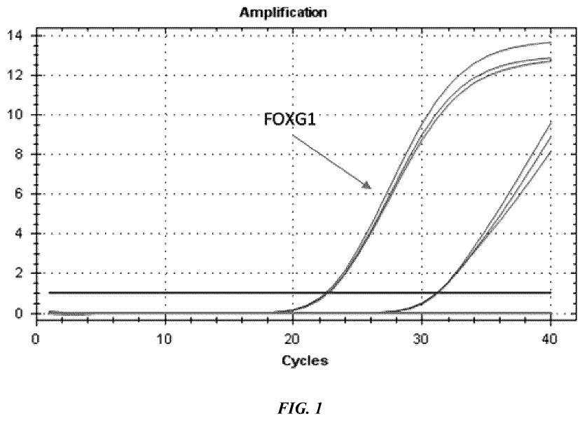

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting. The terminology used herein is for the purpose of describing particular embodiments only and is not intended to be limiting.

The terms “comprise(s),” “include(s),” “having,” “has,” “can,” “contain(s),” “may” and variants thereof, as used herein, are intended to be open-ended transitional phrases, terms, or words that do not preclude the possibility of additional acts or structures.

The singular forms “a,” “an” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

As used herein, “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “antisense molecule” means an oligomeric nucleic acid or oligomeric duplex capable of achieving at least one antisense activity.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety

As used herein, “complementary” in reference to an oligonucleotide means that at least 70%, at 80%, at least 90%, at least 95%, at least 98%, or at least 99% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methylcytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified 2′-deoxyfuranosyl. Thus, the term “MOE gapmer” indicates a gapmer having a sugar motif of 2′-MOE nucleosides in both wings and a gap of 2′-deoxynucleosides. Unless otherwise indicated, an MOE gapmer may comprise one or more modified internucleoside linkages and/or modified nucleobases and such modifications do not necessarily follow the gapmer pattern of the sugar modifications. Table 2, below, provides exemplary MOE-gapmers.

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif include the “5′ wing”, the “gap” and the “3′ wing” which form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise a number of nucleosides selected from 1, 2, 3, 4, 5, 6, 7, 8, 9, 10, or a range that includes or is between any two of the foregoing numbers (e.g., 1-5, 2-7, etc.). In certain embodiments, each nucleoside of each wing of a gapmer is a modified nucleoside.

In certain embodiments, the gap of a gapmer comprises comprise a number of nucleosides selected from 5, 6, 7, 8, 9, 10, 11, 12, 13, 14, 15, 16, 17, 18, 19, 20, 21, 22, 23, 24, 25, or a range that includes or is between any two of the foregoing numbers (e.g., 7-15, 10-20, etc.). In certain embodiments, each nucleoside of the gap of a gapmer is an unmodified 2′-deoxy nucleoside.

In certain embodiments, the gapmer is a deoxy gapmer. In further embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

In another embodiments, modified oligonucleotides comprise, consist essentially of or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

“Inhibit” as used herein refers to the ability to substantially antagonize, prohibit, prevent, restrain, slow, disrupt, alter, eliminate, stop, or reverse the progression or severity of the activity of a particular agent (e.g., FOXG1 AS molecule) or disease.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages can be found in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003); Wan et al., Nuc. Acid. Res. 42, 13456 (2014); Chapter 10 of Locked Nucleic Acid Aptamers in Nucleic Acid and Peptide Aptamers: Methods and Protocols v 535, 2009 by Barciszewski et al., editor Gunter Mayerand; and WO 2017/015555. In certain embodiments, a population of modified oligonucleotides is enriched for modified oligonucleotides having at least one indicated phosphorothioate in the (Sp) configuration.

As used herein, “MOE” means methoxyethyl. “2′-MOE” means a —OCH₂CH₂OCH₃ group at the 2′ position of a furanosyl ring.

A “neurological disease” is any disease that causes electrical, biochemical, or structural abnormalities in the brain, spine, or neurons. For example, a neurological disease may be a neurodegenerative disease. The neurodegenerative disease may result in motor neuron degeneration, for example. The neurological disease may be a FOXG1 syndrome. Symptoms of FOXG1 syndrome that can be alleviated by the methods of the disclosure include autism spectrum disorders (ASD), including, but not limited to, epilepsy, microcephaly (congenital or postnatal), severe intellectual disability, abnormal or involuntary movements, and unexplained episodes of crying.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” or “mC” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 2,6-diaminopurine, 5-hydroxymethylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides have 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a range that includes or is between of any two of the foregoing numbers, linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

“FOXG1 gene” refers to a gene the encodes instructions for making a protein known as forkhead box G1. This protein is a transcription factor, which helps regulate the activity of other genes. Specifically, the forkhead box G1 protein acts as a transcriptional repressor, turning off (repressing) the activity of certain genes when they are not needed. Researchers believe that this protein plays an important role in brain development, particularly in a region of the embryonic brain known as the telencephalon. The telencephalon ultimately develops into several critical structures, including the largest part of the brain (the cerebrum), which controls most voluntary activity, language, sensory perception, learning, and memory.

As used herein a “FOXG1 disease or disorder” or “FOXG1 syndrome” includes various Autism Spectrum Disorders (ASD), Alzheimer's disease and the like. For example, a FOXG1 disease or disorder or syndrome includes phenotypic traits such as epilepsy, microcephaly (congenital or postnatal), severe intellectual disability, abnormal or involuntary movements, and unexplained episodes of crying. The compositions, methods, and kits of the disclosure can be used to treat individuals with FOXG1 syndrome or ASD comprising administering to a subject in need of treatment a therapeutically effective amount of an antisense or inhibitory nucleic acid that inhibits the activity of FOXG1 AS. In some embodiments, the compositions of the disclosure decrease or inhibit the activity of FOXG1 AS. FOXG1 has been shown to antagonize cell cycle reentry by negatively regulating the levels of p21-activated kinase (PAK3) and blocked neuronal apoptosis and Aβ deposition, suggesting that increasing FOXG1 may represent a therapeutic strategy for Alzheimer's disease.

As used herein, “RNAi compound” which includes “inhibitory nucleic acids” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, “sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, —OCN, —CF₃, —OCF₃, —O—C₁₋₁₀ alkoxy, —O—C₁₋₁₀ substituted alkoxy, —O—C₁₋₁₀ alkyl, —O—C₁₋₁₀ substituted alkyl, —S-alkyl, —N(Rₘ)-alkyl, —O-alkenyl, —S-alkenyl, —N(Rₘ)-alkenyl, —O-alkynyl, —S-alkynyl, —N(Rₘ)-alkynyl, —O-alkylenyl-O-alkyl, alkynyl, alkaryl, aralkyl, —O-alkaryl, —O-aralkyl, —O(CH₂)₂SCH₃, —O(CH₂)₂ON(Rₘ)(Rₙ) or —OCH₂C(═O)—N(Rₘ)(Rₙ), where each Rₘ and Rₙ is, independently, H, an amino protecting group, or substituted or unsubstituted C₁₋₁₀ alkyl, and the 2′-substituent groups can be further substituted with one or more substituent groups independently selected from among: hydroxyl, amino, alkoxy, carboxy, benzyl, phenyl, nitro, thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), and alkyl. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the like.

In certain embodiments, a 2′-substituted non-bicyclic modified nucleoside comprises a sugar moiety comprising a non-bridging 2′-substituent group selected from: F, NH₂, N₃, —OCF₃, —OCH₃, —O(CH₂)₃NH₂, —CH₂CH═CH₂, —OCH₂CH═CH₂, —OCH₂CH₂OCH₃, —O(CH₂)₂SCH₃, —O(CH₂)₂ON(Rₘ)(Rₙ), —O(CH₂)₂O(CH₂)₂N(CH₃)₂, and N-substituted acetamide (—OCH₂C(═O)—N(Rₘ)(Rₙ)), where each Rₘ and Rₙ is, independently, H, an amino protecting group, or substituted or unsubstituted C₁₋₁₀ alkyl.

In certain embodiments, a 2′-substituted nucleoside non-bicyclic modified nucleoside comprises a sugar moiety comprising a non-bridging 2′-substituent group selected from: F, —OCF₃, —OCH₃, —OCH₂CH₂OCH₃, —O(CH₂)₂SCH₃, —O(CH₂)₂ON(CH₃)₂, —O(CH₂)₂O(CH₂)₂N(CH₃)₂, and —OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, —OCH₃, and —OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′- (CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof, 4′-C(CH₃)(CH₃)—O-2′ and analogs thereof, 4′-CH₂—N(OCH₃)-2′ and analogs thereof, 4′-CH₂—O—N(CH₃)-2′, 4′-CH₂—C(H)(CH₃)-2′, 4′-CH₂—C(═CH₂)-2′ and analogs thereof, 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b}, is, independently, H, a protecting group, or C₁₋₁₂ alkyl.

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁₋₁₂ alkyl, substituted C₁₋₁₂ alkyl, C₁₋₁₂ alkenyl, substituted C₂₋₁₂ alkenyl, C₂₋₁₂ alkynyl, substituted C₂₋₁₂ alkynyl, C₅₋₂₀ aryl, substituted C₅₋₂₀ aryl, heterocycle radical, substituted heterocycle radical, heteroaryl, substituted heteroaryl, C₅₋₇ alicyclic radical, substituted C₅₋₇ alicyclic radical, halogen, OJ₁, NJ₁₋₂, S J₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁₋₁₂ alkyl, substituted C₁₋₁₂ alkyl, C₂₋₁₂ alkenyl, substituted C₂₋₁₂ alkenyl, C₂₋₁₂ alkynyl, substituted C₂₋₁₂ alkynyl, C₅₋₂₀ aryl, substituted C₅₋₂₀ aryl, acyl (C(═O)—H), substituted acyl, a heterocycle radical, a substituted heterocycle radical, C₁₋₁₂ aminoalkyl, substituted C₁₋₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. patent Publication Nos. Allerson et al., US2008/0039618 and Migawa et al., US2015/0191727.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human subject. The subject or patient may be undergoing other forms of treatment.

As used herein, “target nucleic acid” and “target RNA” mean a nucleic acid that an antisense compound is designed to affect.

A “therapeutically effective amount,” or “effective dosage” or “effective amount” as used interchangeably herein unless otherwise defined, means a dosage of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in an animal, mammal, or human, such as reducing and/or inhibiting the function of a receptor. A therapeutically effective amount may be administered in one or more administrations (e.g., the agent may be given as a preventative treatment or therapeutically at any stage of disease progression, before or after symptoms, and the like), applications or dosages and is not intended to be limited to a particular formulation, combination or administration route. It is within the scope of the present disclosure that the drug may be administered at various times during the course of treatment of the subject. The times of administration and dosages used will depend on several factors, such as the goal of treatment (e.g., treating v. preventing), condition of the subject, etc. and can be readily determined by one skilled in the art.

As used herein, the term “treat” or “treating” a subject, refers to administering a composition or agent described herein to the subject, such that at least one symptom of a disease or disorder is healed, alleviated, relieved, altered, remedied, reduced, ameliorated, or improved. Treating includes administering an amount effective to alleviate, relieve, alter, remedy, reduce, ameliorate, and/or improve one or more symptoms associated with a disease or disorder. The treatment may inhibit deterioration or worsening of a symptom associated with the disease or disorder.

“Haploinsufficiency” or “haploinsufficient” as used herein may refer to when a diploid organism has only a single functional copy of a gene (with the other copy affected by mutation (e.g., expansion, deletion, substitution, etc.) such that it is expressed aberrantly) and the single functional copy does not produce enough of a gene product (typically a protein) to bring about a wild-type condition, leading to an abnormal or diseased state, or it may be altered by some epigenetic mechanism, leading to the total absence of the protein.

To understand the pathophysiological mechanisms of FoxG1 Syndrome (FS) and the electrophysiological profiles of organoids derived from cells having mutations in FoxG1, histological analyses and molecular profiles for three mutations were obtained using Bulk RNAseq and scRNAseq. The analysis showed several altered genes in the mutant brain cortical organoids (BCOs), indicating a possible accelerated maturation in organoid development, an imbalance in the GABAergic and glutamatergic pathways, changes in important genes for Neural Crest Differentiation, hypoplastic dendrites, fewer excitatory synapses, reduced excitatory and genes involved in the MCP2 pathway (a gene involved in Rett syndrome). It is worth noting that patients with mutations in the FOXG1 gene were categorized, until recently, as having Rett syndrome, which may partially explain the clinical phenotypes that converge between the two diseases as impaired development, intellectual disability, and problems with communication and language. The results provided herein show that correction in the mutations in FOXG1 can restore expression of genes involved in MCP2 pathways to normal levels. This suggests that the in vitro model is recapitulating literature descriptions that show that FOXG1 shares common molecular mechanisms with MeCP2 during neuronal development, exhibiting partially overlapping domains of expression in the postnatal cortex and subnuclear neuronal localization.

The disclosure also demonstrates that genes that encode ion channels such as NAV3 are significantly reduced in FOXG1 mutation W308X in BCOs. NAV3 is associated with axon guidance and is highly expressed in the inner cortical plate of the developing cortex, in pyramidal neurons (CA1 hippocampus and somatosensory cortex) and cortical interneurons.

The single-cell transcriptomic results agree with the histological and molecular abnormalities found in FOXG1^{-/+} BCOs from W308X and G224S. Both analyses show that FOXG1 organoids have no FOXG1⁺ cells, and in both mutants, there is an imbalance between GABAergic and glutamatergic neurotransmitters. This imbalance between inhibitory and excitatory pathways, in addition to the subpopulation of cells, may explain one of the phenotypes observed in human brains. The phenotype found in the organoids at the subpopulation level could be a valuable tool for FS modeling and in drug screening.

The levels of FOXG1 in rescue lines rescued through CRISPR engineering were partially restored at approximately half of the value presented by the control BCOs. In view of this result, one of the hypotheses speculated in this work was to verify if the expression at some time point could be monoallelic, and if so, to verify if epigenetic alterations that may be negatively modulating the other allele. This is because theoretically, one would expect the same half of FOXG1 expression as obtained from the wild-type allele. Therefore, it was hypothesized that mechanisms that have been discovered for other neurological diseases may also be similar in FS. Thus, as in Angelman syndrome, the inactive allele may be imprinted at some stage of embryonic neurodevelopment and that would explain the unexpected result.

Experiments were performed to research epigenetic mechanisms that may be associated with FOXG1 such as methylation (since the genetic region is located within a CpG island), acetylation and the antisense transcript, called FOXG1-AS. The disclosure demonstrates that treatments with 5-AZAD were able to increase FOXG1 only in early periods, that is, before 3 weeks. It is hypothesized that such demethylation acts in dividing cells, this type of modulation would only occur in more proliferative phases.

One approach using sodium butyrate as an HDAC inhibitor seemed more interesting since it was able to positively modulate FOXG1 in older organoids (3-4 months). These results suggest that acetylation may be a mechanism to upregulate FOXG1. Another therapeutic approach tested was using ASOs as antisense transcript silencing molecules; this approach proved to be efficient. The ASO structure was designed with a 5-10-5 pattern (2′MOE) and all nucleotides with phosphonothioate bonds. One of the ASOs, for example, targets exon 4 of the antisense transcript and was able to increase levels of FOXG1 transcripts by 50% in 4-month organoids.

The disclosure shows that a mechanism to regulate the expression of FOXG1 in humans includes using ASOs as set forth in Tables 1 and 2, which can increase the expression of FOXG1 in neural cells through the reduction or removal of FOXG1-AS. It acts by targeting either exonic, intronic, 3′ or 5′ regions of the FOXG1-AS. Examples of ASO designed to target such regions are provided herein. The disclosed ASOs are capable of positively modulating the expression of FOXG1 through hybridization with FOXG1-AS. The functional ASOs described here can increase FOXG1 expression by at least 10%, 20%, 30%, 40%, 50%, 55%, 60%, 65%, 70%, 75%, 80%, 85%, 90%, 95%, 98% or 100% compared to the level of FOXG1 expression in saline-treated organoid cells (mini-brains) or an untargeted ASO (hereinafter referred to as a scrambled ASO).

The disclosure also demonstrates that the FOXG1 gene is regulated by methylation upstream to its promoter under a CpG island. Upon de-methylation, using reagents such as the ASOs described above or 5-AZAD, it is possible to up-regulate the FOXG1 gene.

Disclosed herein are methods of treatment that may comprise administering to a subject in need thereof a composition comprising an effective amount of one or more antisense oligonucleotides or inhibitor oligonucleotides that treats neurological diseases by inhibiting FOXG1 AS activity. Such treatment may further be in combination with demethylating agents and/or modulators of acetylation (e.g., histone deacetylase inhibitors).

The disclosure provides oligonucleotides (modified or unmodified) that can be used to modulate FOXG1 AS activity (see Table 1). It should be noted that the sequence of Table 1 are DNA and that corresponding RNA sequences are contemplated wherein T is replaced with U in the sequences of Table 1.

TABLE 1
(5′ to 3′) generic sequences useful in designing
FOXG1 AS antisense or inhibitory nucleic acids.

| Sequence | | |
|---|---|---|
| ID | | |
| number | Sequence | position |
| 1 | ATTACATTTATTCTGCTCCC | Exon4-intron4 |
| 2 | GACTTAAATGCAGGGAAGAG | Exon4 (ASO4) |
| 3 | TACTCCAATTACACAAACCC | Intron1 |
| 4 | CAATTCATTAGAAATGGGTTCCC | 3′ |
| 5 | TGCCCCAGCTATTATCTGAG | Exon4 |
| 6 | CAGTTGTGAGTTATTAGCTG | Exon4 |
| 7 | CTGTCTTTAAGTTATGGCCA | Exon4 |
| 8 | ATCCTCATTGAGGCTTGGGAA | Exon3 |
| 9 | AAAATGTGGTATGTTTCGTGC | Exon1 |
| 10 | AGTGTGGCTTTATATACCC | Exon2 |
| 11 | TAAGAAGGGTCTAAGGAGG | Exon4 |
| 12 | AACATTTACTGCATGCCAGT | Exon4 |
| 13 | TCACAGAACATTTTAAAGA | Exon4 |
| 14 | TCAGGCTGTGAGTAAGAGAG | Exon2-Exon3 |

In one embodiment, the disclosure provides modified oligonucleotides comprising 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a range that includes or is between of any two of the foregoing numbers, linked nucleosides. and having a nucleobase sequence comprising at least 8, at least 9, at least 10, at least 11 at least 12, at least 13, at least 14, at least 15, at least 16, at least 17, at least 18, at least 19 or at least 20 consecutive nucleotide bases of any of the nucleobase sequences of SEQ ID NO:1-14 in Table 1. In some embodiments, the modified oligonucleotide is at least 80% to 100% (i.e., 80%, 82%, 84%, 86%, 88%, 90%, 92%, 94%, 96%, 98% or 100%; or any numerical range or value between any of the foregoing values) identical to any of the sequences comprising or consisting of SEQ ID NO:1-14.

The sequences provided in Table 1 can be used to design antisense molecules for inhibition of FOXG1 AS activity. For example, gapmer oligonucleotides can be designed using the sequences in Table 1 and can comprise a 5′-wing of about 3-5 nucleotides, a 3′-wing of about 3-5 nucleotides and a gap region comprising 8-12 consecutive deoxyribonucleosides of any one of the sequences of Table 1. In one embodiment, an oligonucleotide of the disclosure comprises a gapmer having a gap segment of at least 8, at least 9, at least 10, at least 11 at least 12, at least 13, at least 14, at least 15, at least 16, at least 17, at least 18, at least 19 or at least 20 consecutive nucleotide bases of any of the nucleobase sequences of SEQ ID NO:1-14 in Table 1; flanked by a 5′ and 3′ wing segments, wherein the gap segment is located between the 5′ and 3′ wing segments and wherein each of the wing segments comprises a modified sugar. In one embodiment, the gap segment is 8-10 nucleosides in length and each wing segment is 3-5 modified nucleosides in length. In yet another embodiment, an oligonucleotide of the disclosure comprises a 5′ wing segment comprising modified sugars and having the nucleobase sequence of the first 3-5 nucleobases of any of SEQ ID NO:1-14, followed by a gap of the next 8-12 unmodified nucleotides of the same sequence corresponding to SEQ ID NO:1-14, followed by a 3′ wing segment comprising modified sugars and having the nucleobase sequence of the last 3-5 nucleobases of the same sequence corresponding to SEQ ID NO:1-14. Table 2 provides MOE gapmers of the disclosure.

The 5′ and/or 3′ wings can comprise the following chemistries: 2′-OMe, 2′-MOE, LNA or DNA, by themselves or used in combination with one another. The backbone linkage of the 5′ and/or 3′ wings can be phosphorothioate or a mixture of phosphodiester and phosphorothioate. Any combination of phosphorothioate and phosphodiester linkages can be found throughout the wing and gap regions (i.e., any position in the oligo can have either a phosphorothioate or phosphodiester linkage).

In some embodiments, the oligonucleotide is single stranded. In some embodiments the oligonucleotide comprises or is complexed with a moiety that neutralizes charge on the oligonucleotide to promote uptake and transfer across a cell membrane.

TABLE 2
FOXG1 AS Antisense Oligonucleotide Sequences (ASOs).
(Gapmer design: 5′- five 2′-methoxyethylribose nucleotides - ten DNA nucleotides - five 2′-
methoxyethylribose nucleotides - 3′; /i2MOErN/ = 2′-methoxyethylribose nucleotide; A, C,
T, G = adenine, cytosine, thymine, guanosine, respectively; * = phosphorothioate
linkages)(Note that the following table provide 2′MOE wings, however, alternative wings
comprising 2-OMe, LNA, Affinity + locked nucleic acids (see Integrated DNA Technologies
(Coralville, Iowa, U.S.A.) etc. are contemplated)

| # | ASOs | Position |
|---|---|---|
| 1 | /52MOErG/*/i2MOErG/*/i2MOErC/*/i2MOErA/*/i2MOErT/*A | Gene |
| | *T*G*C*A*G*A*T*A*A*/i2MOErT/*/i2MOErG/*/i2MOErT/* | MALAT1 |
| | /i2MOErT/*/32MOErC/ | |
| 2 | /52MOErT/*/i2MOErA/*/i2MOErG/*/i2MOErT/*/i2MOErG/*C | FOXG1-AS |
| | *G*G*A*C*C*T*A*C*C*/i2MOErC/*/i2MOErA/*/i2MOErC/* | Scramble |
| | /i2MOErG/*/32MOErA/ | |
| 3 | /52MOErA/*/i2MOErT/*/i2MOErT/*/i2MOErA/*/i2MOErC/*A | FOXG1-AS |
| | *T*T*T*A*T*T*C*T*G*/i2MOErC/*/i2MOErT/*/i2MOErC/*/ | exon4-intron4 |
| | i2MOErC/*/32MOErC/ | |
| 4 | /52MOErG/*/i2MOErA/*/i2MOErC/*/i2MOErT/*/i2MOErT/*A | FOXG1-AS |
| | *A*A*T*G*C*A*G*G*G*/i2MOErA/*/i2MOErA/*/i2MOErG/ | exon 4 |
| | */i2MOErA/*/32MOErG/ | |
| 5 | /52MOErT/*/i2MOErA/*/i2MOErC/*/i2MOErT/*/i2MOErC/*C | FOXG1-AS |
| | *A*A*T*T*A*C*A*C*A*/i2MOErA/*/i2MOErA/*/i2MOErC/* | Intron 1 |
| | /i2MOErC/*/32MOErC/ | |
| 6 | /52MOErC/*/i2MOErA/*/i2MOErA/*/i2MOErT/*/i2MOErT/*C | FOXG1-AS |
| | *A*T*T*A*G*A*A*A*T*G*G*G/i2MOErT/*/i2MOErT/*/ | 3′ |
| | i2MOErC/*/i2MOErC/*/32MOErC/ | |
| 7 | /52MOErT/*/i2MOErG/*/i2MOErC/*/i2MOErC/*/i2MOErC/*C | FOXG1-AS |
| | *A*G*C*T*A*T*T*A*T*/i2MOErC/*/i2MOErT/*/i2MOErG/*/ | exon 4 |
| | i2MOErA/*/32MOErG/ | |
| 8 | /52MOErC/*/i2MOErA/*/i2MOErG/*/i2MOErT/*/i2MOErT/*G | FOXG1-AS |
| | *T*G*A*G*T*T*A*T*T*/i2MOErA/*/i2MOErG/*/i2MOErC/* | exon 4 |
| | /i2MOErT/*/32MOErG/ | |
| 9 | /52MOErC/*/i2MOErT/*/i2MOErG/*/i2MOErT/*/i2MOErC/*T | FOXG1-AS |
| | *T*T*A*A*G*T*T*A*T*/i2MOErG/*/i2MOErG/*/i2MOErC/* | exon 4 |
| | /i2MOErC/*/32MOErA/ | |
| 10 | /52MOErA/*/i2MOErT/*/i2MOErC/*/i2MOErC/*/i2MOErT/*C | FOXG1-AS |
| | *A*T*T*G*A*G*G*C*T*T*/i2MOErG/*/i2MOErG/*/i2MOErG/ | EXON 3 |
| | */i2MOErA/*/32MOErA/ | |
| 11 | /52MOErA/*/i2MOErA/*/i2MOErA/*/i2MOErA/*/i2MOErT/* | FOXG1-AS |
| | G*T*G*G*T*A*T*G*T*T*T*/i2MOErC/*/i2MOErG/*/i2MOErT/ | Exon1 |
| | */i2MOErG/*/32MOErC/ | |
| 12 | /52MOErA/*/i2MOErG/*/i2MOErT/*/i2MOErG/*/i2MOErT/*G | FOXG1-AS |
| | *G*C*T*T*T*A*T*A*/i2MOErT/*/i2MOErA/*/i2MOErC/*/ | Exon2 |
| | i2MOErC/*/32MOErC/ | |
| 13 | /52MOErT/*/i2MOErA/*/i2MOErA/*/i2MOErG/*/i2MOErA/* | FOXG1-AS |
| | A*G*G*G*T*C*T*A*A*/i2MOErG/*/i2MOErG/*/i2MOErA/*/ | Exon4 |
| | i2MOErG/*/32MOErG/ | |
| 14 | /52MOErA/*/i2MOErA/*/i2MOErC/*/i2MOErA/*/i2MOErT/*T | FOXG1-AS |
| | *T*A*C*T*G*C*A*T*G*/i2MOErC/*/i2MOErC/*/i2MOErA/* | Exon4 |
| | /i2MOErG/*/32MOErT/ | |
| 15 | /52MOErT/*/i2MOErC/*/i2MOErA/*/i2MOErC/*/i2MOErA/*G | FOXG1-AS |
| | *A*A*C*A*T*T*T*T*/i2MOErA/*/i2MOErA/*/i2MOErA/*/ | Exon4 |
| | i2MOErG/*/32MOErA/ | |
| 16 | /52MOErT/*/i2MOErC/*/i2MOErA/*/i2MOErG/*/i2MOErG/*C | FOXG1-AS |
| | *T*G*T*G*A*G*T*A*A*/i2MOErG/*/i2MOErA/*/i2MOErG/ | Exon2- |
| | */i2MOErA/*/32MOErG/ | exon3 |

The FOXG1 AS antisense or inhibitory nucleic acids of the disclosure can inhibit the activity associated with FOXG1 AS. The FOXG1 AS antisense or inhibitory nucleic acids can include any combination of the oligonucleotides set forth in Table 2 and sequences that are 98%-99% identical thereto.

Methods of treatment may include any number of modes of administering a disclosed composition or compound. Modes of administration may include aqueous, lipid, oily or other solutions, emulsions such as oil-in-water emulsions, liposomes, aqueous or oily suspensions and the like. Typically, an ASO of the disclosure will be administered directly to the CNS of the subject. Accordingly, the formulation or composition will be sterile and more preferably be suitable for injection. The following formulations and methods are merely exemplary and are in no way limiting.

Formulations suitable for parenteral administration include aqueous and non-aqueous, isotonic sterile injection solutions, which may contain anti-oxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives. The formulations may be presented in unit-dose or multi-dose sealed containers, such as ampules and vials, and may be stored in a freeze-dried (lyophilized) condition requiring only the addition of the sterile liquid excipient, for example, water, for injections, immediately prior to use. Extemporaneous injection solutions and suspensions may be prepared from sterile powders, granules, and tablets.

Additional therapeutic agent(s) may be administered simultaneously or sequentially with the disclosed one or more antisense or inhibitory nucleic acids and compositions (such as, e.g., sodium butyrate, 5-AZAD, Tichostatin-A (TSA) or similar compounds of either of the foregoing). Sequential administration includes administration before or after the disclosed one or more antisense or inhibitory nucleic acids or compositions. In some embodiments, the additional therapeutic agent or agents may be administered in the same composition as the disclosed one or more antisense or inhibitory nucleic acids. In other embodiments, there may be an interval of time between administration of the additional therapeutic agent and the disclosed one or more antisense or inhibitory nucleic acids. In some embodiments, administration of an additional therapeutic agent with a disclosed one or more antisense or inhibitory nucleic acids may allow lower doses of the other therapeutic agents and/or administration at less frequent intervals. When used in combination with one or more other active ingredients, the one or more antisense or inhibitory nucleic acids of the disclosure and the other active ingredients may be used in lower doses than when each is used singly. Accordingly, the pharmaceutical compositions of the disclosure include those that contain one or more other active ingredients, in addition to one or more antisense or inhibitory nucleic acids of the disclosure. The above combinations include combinations of one or more antisense or inhibitory nucleic acids of the disclosure not only with one other active compound, but also with two or more other active compounds. For example, the compound of the disclosure may be combined with a variety of drugs to treat neurological diseases.

The disclosed one or more antisense or inhibitory nucleic acids can be combined with the following, but are not limited, anticholinergic drugs, anticonvulsants, antidepressants, benzodiazepines, decongestants, muscle relaxants, pain medications, and/or stimulants. Additional types of therapy and treatment include, but are not limited to digital communication devices, feeding tubes, mechanical ventilation, nutritional support, deep brain stimulation, occupational therapy, physical therapy, and/or speech therapy.

The disclosed composition(s) may be incorporated into a pharmaceutical composition suitable for administration to a subject (such as a patient, which may be a human or non-human). The pharmaceutical compositions may comprise a carrier (e.g., a pharmaceutically acceptable carrier). Any suitable carrier can be used within the context of the disclosure, and such carriers are well known in the art. The choice of carrier will be determined, in part, by the particular use of the composition (e.g., administration to an animal) and the particular method used to administer the composition. Accordingly, there is a wide variety of suitable formulations of the composition of the present invention.

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of one or more antisense or inhibitory nucleic acids of the disclosure are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants may also be present in the composition, according to the judgment of the formulator.

The route by which the disclosed one or more compounds or compositions of the disclosure are administered and the form of the composition will dictate the type of carrier to be used.

The pharmaceutical compositions of the disclosure can be administered in a number of ways depending upon whether local or systemic treatment is desired and upon the area to be treated. Administration can be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal, intra-cerebroventricular, or intraventricular, administration. In one embodiment the antisense or inhibitory nucleic acid is administered IV, IP, orally, topically or as a bolus injection or administered directly in to the target organ. In another embodiment, the antisense or inhibitory nucleic acid is administered intrathecal or intra-cerebroventricular as a bolus injection.

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The percentage of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of Theobroma. The percentage of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The percentage of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The percentage of disintegrant(s) in a systemic composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The percentage of flavor(s), when used, in a systemic or topical composition is typically about 0.1 to about 1.0%.

Suitable antioxidants include butylated hydroxyanisole (“BHA”), butylated hydroxytoluene (“BHT”), and vitamin E. The percentage of antioxidant(s) in a systemic or topical composition is typically about 0.1 to about 5%.

Suitable preservatives include benzalkonium chloride, methyl paraben and sodium benzoate. The percentage of preservative(s) in a systemic or topical composition is typically about 0.01 to about 5%.

Suitable glidants include silicon dioxide. The percentage of glidant(s) in a systemic or topical composition is typically about 1 to about 5%.

Suitable solvents include water, isotonic saline, ethyl oleate, glycerin, hydroxylated castor oils, alcohols such as ethanol, and phosphate buffer solutions. The percentage of solvent(s) in a systemic or topical composition is typically from about 0 to about 100%.

Suitable suspending agents include AVICEL RC-591 (from FMC Corporation of Philadelphia, PA) and sodium alginate. The percentage of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and Mccutcheon's Volume 1, Emulsifiers & Detergents, 1994, North American Edition, pp. 236-239. The percentage of surfactant(s) in the systemic or topical composition is typically about 0.1% to about 5%.

Compositions and formulations for parenteral, intrathecal, intra-cerebroventricular, or intraventricular administration can include sterile aqueous solutions which can also contain buffers, diluents and other suitable additives such as, but not limited to, penetration enhancers, carrier compounds and other pharmaceutically acceptable carriers or excipients. For example, an intrathecal cerebrospinal fluid (CSF) catheter can be used to deliver antisense formulations of the disclosure. The catheter can be inserted at the L3 or L4 vertebrae. The distal tip of the catheter extends within the intrathecal space to approximately the L1 vertebrae. Antisense oligonucleotides are dissolved in saline, are sterilized by filtration, and are administered at 0.33 mL/min in a 1.0 mL volume followed by a 0.5 mL sterile water flush. Total infusion time is 4.5 min.

Although the amounts of components in the systemic compositions may vary depending on the type of systemic composition prepared, in general, systemic compositions include 0.01% to 50% of active compound and 50% to 99.99% of one or more carriers. Compositions for parenteral administration typically include 0.1% to 10% of actives and 90% to 99.9% of a carrier including a diluent and a solvent.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

In vivo testing of candidate antisense or inhibitory nucleic acids may be conducted by means known to one of ordinary skill in the art with reference to the screening methodology and examples described herein. For example, the candidate one or more antisense or inhibitory nucleic acids may be administered to a mammal, such as a mouse, rat, pig, dog or a rabbit. The mammal may be administered, by any route deemed appropriate, a dose of a candidate antisense or inhibitory nucleic acids. Conventional methods and criteria can then be used to monitor animals for signs of effects on FOXG1 AS activity or expression. If needed, the results obtained in the presence of the candidate antisense or inhibitory nucleic acids can be compared with results in control animals that are not treated with the candidate antisense or inhibitory nucleic acids. Dosing studies may be performed in, or in conjunction with, the herein described methods for identifying one or more antisense or inhibitory nucleic acids capable of treating a neurological disease or disorder and/or any follow-on testing of candidate antisense or inhibitory nucleic acids in vivo. One of skill in the art of medicine may determine the appropriate dosage of one or more antisense or inhibitory nucleic acids. The dosage may be determined by monitoring the subject for signs of disease inhibition or amelioration. The dosage may be increased or decreased to obtain the desired frequency of treatment. The toxicity and efficacy of one or more antisense or inhibitory nucleic acids may be determined by standard pharmaceutical procedures in cell cultures or experimental animals, e.g. determining the lethal dose to 50% of the population (LD50) and the dose therapeutically effective in 50% of the population (ED50). The dose ratio of LD50/ED50 is the therapeutic index and, indicating the ratio between the toxic and therapeutic effects. A delivery system may be designed to help prevent toxic side effects, by delivering the one or more antisense or inhibitory nucleic acids to specific targets, e.g., delivered specifically to motor or central nervous system neurons. The optimal dose of the one or more antisense or inhibitory nucleic acids may be determined based on results of clinical electrophysiology or electromyography to analyze excitability in peripheral nerves, for example.

The dosage for use in humans may be determined by evaluating data obtained from animal studies and cell culture assays. The preferred dosage will have little or no toxicity and include the ED50. The dosage may vary depending on the dosage form and route of administration. For any antisense or inhibitory nucleic acid used in the methods described herein, the dosage may be estimated initially in cell culture. A dose may be formulated in animal models that includes the concentration of the test compound which achieves a half maximal inhibition of symptoms (LD50) as determined in cell culture. Such information obtained from cell cultures and animal models may be used to more accurately determine useful doses in humans.

The present invention has multiple aspects, illustrated by the following non-limiting examples.

### EXAMPLES

### Example 1

### Model for Studying FOXG1 Syndrome

In order to model the FOXG1 syndrome (FS) in vitro, a protocol was developed to produce a three-dimensional, scaled-down model of the human brain, capable of mimicking several developmental characteristics at the cellular and molecular levels of a human brain. Patients with FS were selected based on clinical and molecular diagnostics information, including the types of FOXG1 mutation they carry. For patients, the details of each FOXG1 mutation was confirmed via directing Sanger sequencing of the FOXG1 locus in iPSC. Control subjects were apparently people, who had no history of psychiatric or genetic disorders.

Reprogramming of Skin Fibroblasts Into iPSCs

Skin fibroblasts were obtained from biopsies taken from FOXG1 and control subjects, followed by culturing in DMEM/F12 medium containing 10% fetal bovine serum and penicillin/streptomycin, iPSCs were derived from fibroblasts via cellular reprogramming, as described in Marchetto et al. 41. Briefly, fibroblast cultures were transduced with Sendai viruses containing over-expression cassettes for OCT4, SOX2, KLF4, and MYC (Cytotune iPS 2.0 Sendai reprogramming kit; Thermo Fisher Scientific). Seven days after transduction, cells were re-plated onto a feeding layer composed of murine embryonic fibroblasts (mEFs) in DMEM/F12 containing 20% Knockout Serum Replacement (Thermo Fisher Scientific), 1% non-essential amino acids (NEAA), and 100 μM β-mercaptoethanol, iPSC colonies were identified after 2 weeks and transferred to 6 cm plates coated with Matrigel (BD Biosciences), after which time they were maintained in mTeSR1 Plus medium (Stem Cell Technologies) and passaged by manual picking with the aid of a pipette tip. All iPSC clones were then passaged until P10 and 2 clones were chosen for organoid derivation after this passage. The analyzed clones were verified about the presence of unwanted chromosomal abnormalities through the karyotype technique. Results provided herein are from experiments conducted with one or two P15 iPSC clones per subject, and confirmation of consistency in the observed phenotypes was obtained from 2 independent iPSC clones per subject. Culture were tested every two weeks for mycoplasma and if contaminated the cultures were discarded.

Validation of iPSC clones was performed via immunostaining for SOX2, OCT4, NANOG, and LIN28. Briefly, a total of 20 colonies were grown inside wells of LabTek II 8-well chambered slides (Thermo Fisher Scientific) until they reached a diameter of 2 mm. Colonies were then fixed with 4% paraformaldehyde solution for 10 min, washed once with 1× Phosphate Buffered Saline (PBS), permeabilized with 1% Triton X-100 for 5 min, washed again in 1×PBS, and blocked with 10% Bovine Serum Albumin (BSA)/1% Triton X-100/1×PBS. Incubation with primary antibodies was performed in the same blocking solution for 16 h at 4° C. Primary antibodies used were rabbit anti-SOX2 (Abcam; ab97959; 1:1000), rabbit anti-OCT4 (Abcam; ab19857; 1:100), rabbit anti-NANOG (GeneTex; GTX100863; 1:100), and rabbit anti-LIN28 (Cell Signaling; 3978; 1:500). After 3 washes in 1×PBS, colonies were incubated with fluorescently labeled secondary antibodies for 3 h, and nuclei were counterstained with 1 μg/mL DAPI (Thermo Fisher Scientific) for 30 min. Slides were mounted with ProLong Gold anti-fading solution (Thermo Fisher Scientific).

IPSCs were used to generate cortical organoids that exhibit an evolving cellular transcriptional profile over several months. For the development of organoids, iPSCs were fed every other day with mTeSR1 Plus for 7 days. Colonies were dissociated using Accutase (Life Technologies) in preheated (37° C.) PBS (1:1) (5 μM ROCK inhibitor) for 10-20 minutes at 37° C. Cells were filtered on a 40 uM filter and centrifuged for 4 minutes at 100×g. The cell pellet was resuspended in mTeSR1 Plus supplemented with 10 μM SB431542 (SB; Stemgent, Cambridge, MA, USA), 5 μM ROCK inhibitor (Y-27632; Calbiochem, Sigma-Aldrich, St. Louis, MO, USA) and 1 μM Dorsomorphin (Dorso; R&D Systems, Minneapolis, MN, USA). Approximately 4×10⁶ cells were transferred to one well of a 6-well plate and kept in suspension under rotation (95 rpm). After 24 hours, the medium was changed for the next 2 days, using the same medium as before, except ROCK inhibitor. After 3 days, mTeSR1 plus was substituted by Medial [Neurobasal (Life Technologies) supplemented with GlutaMAX, 1% Gem21 NeuroPlex (Gemini Bio-Products), 1% N2 NeuroPlex (Gemini Bio-Products), 1% NEAA (Life Technologies), 1% PS (Life Technologies), 10 μM SB and 1 μM Dorso] for 7 days. Then, the cells were maintained in Media2 [Neurobasal with GlutaMAX, 1% Gem21 NeuroPlex, 1% NEAA, and 1% PS] supplemented with 20 ng/ml FGF2 (Life Technologies) for 7 days, followed by 7 additional days in Media2 supplemented with 20 ng/mL of FGF2 and 20 ng/ml EGF (PeproTech, Rocky Hill, NJ, USA). Next, cells were transferred to Media3 [Media2 supplemented with 10 ng/ml of BDNF, 10 ng/ml of GDNF, 10 ng/ml of NT-3 (all from PeproTech), 200 μM L-ascorbic acid and 1 mM dibutyryl-cAMP (Sigma-Aldrich) to promote maturation, gliogenesis and activity]. After 7 days, cortical organoids were maintained in Media2 for as long as needed, with media changes every 3-4 days.

### Immunofluorescence Staining

BCOs were washed with PBS 1×, fixed with 4% paraformaldehyde for 4 hours at 4° C. and cryoprotected in 30% sucrose for 24-48 hs. Brain Cortical Organoids (BCOs) were then embedded in CRYO-OCT COMPOUND 40Z Tissue-Tek O.C.T. To produce 20 μm sections on a Leica CM1850 cryostat. Slides were air-dried for 10 min, permeabilized in 0.2% Triton X-100/1×PBS for 15 min, and blocked with 0.1% Triton X-100/2% BSA/1×PBS for 45 min at 25° C. The slides were incubated using primary antibodies in the same solutions, overnight at 4° C. Primary antibodies used were: chicken anti-MAP2 (Abcam; ab5392; 1:1000); rabbit anti-SOX2 (Cell Signaling Technology; 2748; 1:500); rabbit anti-CC3 (Cleaved Caspase 3) (Cell Signaling; 9664 S; 1:500); mouse anti-Nestin (Abcam; ab22035; clone [10C2]; 1:1000); rabbit anti-FOXG1 (Abcam; ab196868; 1:500); After incubation in a solution containing primary antibodies, slides were washed three times in 1×PBS, for 5 min each, and incubated with fluorescently labeled secondary antibodies (Alexa Fluor 488- or 555-conjugated antibodies; 1:2000 dilution; Thermo Fisher Scientific) in the same type of solution as primary antibodies, for 45 min at 25° C. After further washes in 1×PBS, slides were counterstained with DAPI solution (1 μg/mL) for 5 min and mounted with ProLong Gold. For NPCs, these cells were seeded at a density of 10,000 cells per well of a 96-well plate. They were fixed in PFA 4% at 80% of confluence and incubated with primary antibodies rabbit anti-FOXG1 (Abcam; ab196868; 1:500) followed DAPI solution (1 μg/mL) for 5 min and mounted with ProLong Gold. All images were taken using a Zeiss fluorescence microscope equipped with Apotome (Axio Observer Apotome, Zeiss).

### Real-Time Quantitative PCR

RNeasy Mini Plus kit (Qiagen) was used for RNA extraction. For samples with FOXG1 analysis, DNase I treatment was used on the column, as per the manufacturer's recommendations. A total of 0.5-1 micrograms of RNA were reverse transcribed into cDNA using the Superscript III First-Strand Reverse Transcription System (Thermo Fisher Scientific). For real-time quantitative PCR (RT-qPCR) pre-validated FAM-MGB TaqMan probes (Thermo Fisher Scientific) and Applied Biosystems TaqMan Fast Advanced Master Mix (cat. 4444557) was used on a CFX Connect Real-Time PCR detection system with a Maestro software (Bio-Rad; version 1.1), with the following cycling parameters: 94° C. for 3 min, followed by 40 cycles of 94° C. for 30 s and 68° C. for 1 min. The following TaqMan probes were used: FOXG1 (Hs01850784_s1), FOXG1-AS (Hs03879706_s1), FOXG1-AS (Hs03879706_s1), GAPDH (Hs99999905_m1) and TBP (Hs00427620_m1). For analysis with SYBER, the following primers were used: FOXG1 (Forward: CCCTCTACTGGCCCATGTC (SEQ ID NO:20) and Reverse: GTGGAGAAGGAGTGGTTGTTG (SEQ ID NO:21)), TBP (Forward: GAACCACGGCACTGATTTTC (SEQ ID NO:22) and Reverse: CCCCACCATGTTCTGAATCT (SEQ ID NO:23)) GAPDH (Forward: GTCTCCTCTGACTTCAACAGCG (SEQ ID NO:24) and reverse: ACCACCCTGTTGCTGTAGCCAA (SEQ ID NO:25)). The temperatures were: 95° C. for 10 min, followed by 40 cycles of 95° C. for 15 s and 60° C. for 30 min. All RT-qPCR analyses were conducted using RNA extracted from at least 2 independent biological samples per subject/condition, with 2 or 3 technical replicates for each probe set/sample combination. Normalization was achieved with endogenous control genes TBP or GAPDH, and relative expression was calculated using the traditional ΔΔCt method.

### Genomic and Messenger RNA Sequencing

For genome sequencing FOXG1 gene was amplified in two fragments using Neb fusion hi-fidelity in a final volume of 20 uL using two pair primers. Pair 1: Forward: GAAAATGATCCCCAAGTCCTC (SEQ ID NO:26) and Reverse: GAGTCAACACGGAGCTGTAGG (SEQ ID NO:27)). Pair 2: CGAGAAGAAGAACGGCAAGTA (SEQ ID NO:28) and Reverse GTGGAGAAGGAGTGGTTGTTG (SEQ ID NO:29)). For the first pair, the following temperatures were used: 98° C.-30 sec, 98° C.-10 sec, 64° C.-30 sec, 72° C.-45 sec (35×), 72° C.-7 min. For pair 2: 98° C.-30 sec, 98° C.-10 sec, 64° C.-20 sec, 72° C.-15 sec (40×) and 72° C.-2 min. The PCR amplifications generated two fragments of 1022 bp and 558 bp, respectively. For RNA sequencing, total RNA was used. For each subject, RNA was extracted from 2 independently prepared biological replicates and analyzed in 2 replicates. Analysis of the presence of genomic DNA was performed for all samples submitted to reverse transcription reaction in the absence of reverse transcriptase enzyme. The electropherograms were obtained through analysis via Sanger Sequencing and analyzed using Geneious Prime v.2022.2.1.

### BCO Bulk RNA-Seq

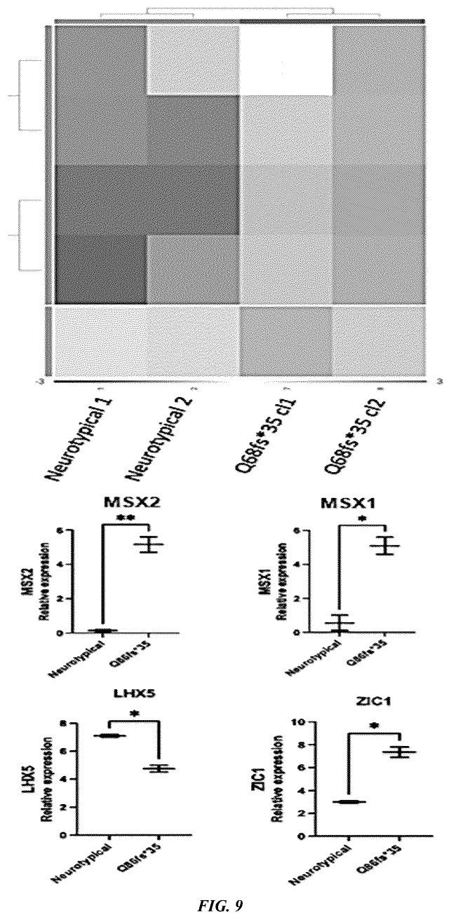

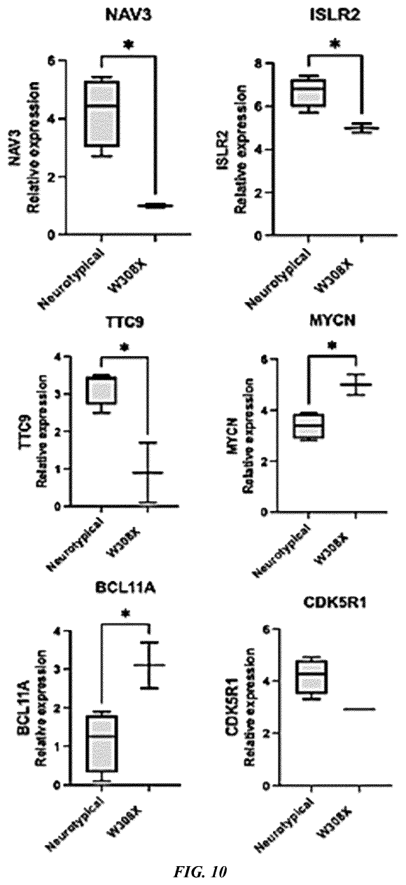

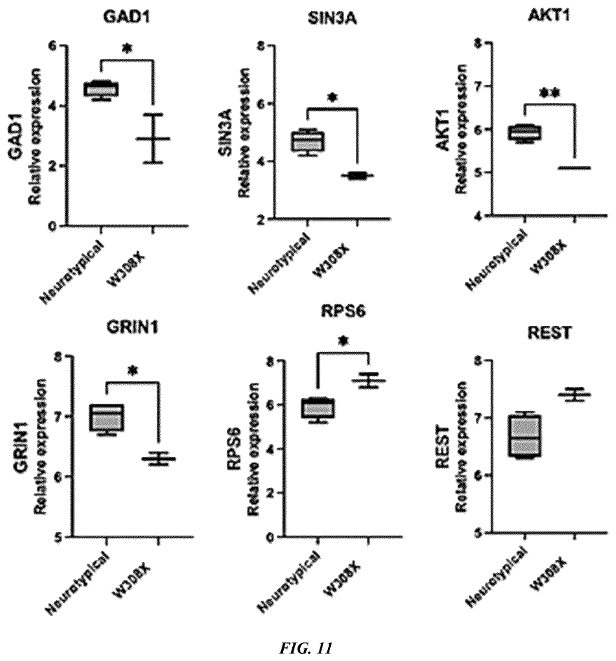

Bulk RNA-Seq BCOs was performed at 4 months on 8 libraries, from 2 clones for W308X, 2 clones for q86FS*35, 2 clones for W308X rescue lines, and 2 different Wild type for controls. RNA was extracted using the RNeasy Mini Plus kit (Qiagen) on 20 organoids. RNA was submitted to Novaseq S4, run type PE100. To estimate transcript-level expression from bulk RNA-Seq data, Rosalind software was used. Pairwise differential expression (DE) was performed with fold change <1.5 or >1.5 and p-value <0.05. The results were plotted in volcano plots, histograms, and independent graphs according to FIGS. 9, 10 and 11.

### Cortical NPC Differentiation

The Neuro progenitor cells (NPC) for Q86fs*35 mutants were generated using iXcell protocol. The NPC was generated from cortical cells derived from iPSC.

Transgenic FOXG1 iPSC Lines.

Homozygous and heterozygous cell lines for the FOXG1 gene were established. Cells were generated and characterized under catalog numbers GM27881 and GM27882 (Coriell Institute; [https]://catalog.coriell.org), respectively.

### Methods of Treating FOXG1 Syndrome via the Use of Specific Oligonucleotides

Long non-coding RNAs (lncRNAs) are defined as a subgroup of non-coding RNA molecules that consist of at least 200 nucleotides and have no or limited protein-coding capacity. Natural antisense transcripts can regulate gene expression through epigenetic mechanisms (DNA methylation, chromatin modifications, and monoallelic expression (e.g., genomic imprinting, X-chromosome inactivation, and random monoallelic deletion of autosomal loci). Antisense transcripts can bind to the corresponding DNA strand, resulting in DNA methylation or providing a scaffold for histone-modifying enzyme (HME) recruitment and subsequent changes in chromatin status. Given this, it was speculated that the non-coding long antisense RNA for the FOXG1 gene (FOXG1-AS) can modulate the expression of the FOXG1 gene. FOXG1-SA is close to the FOXG1 gene but in the opposite direction.

One strategy that was explored for reducing the presence of FOXG1-AS was through the use of Antisense Oligonucleotides (ASOs). ASOS are 15 to 25 nt DNA sequences designed to bind to and degrade complementary RNA targets. ASOs have been used to study the role of long non-coding RNAs (lncRNAs) in gene regulation and drug therapies. To validate the action of ASOs on organoids, test were performed on concentrations and the need to use liposomes in vitro tests. To assess the rates of mRNA loss in the presence or absence of ASOs, wild-type organoids (3 months) were treated with ASOs targeting nuclear RNAs (MALAT1 lncRNA) for 36 hours (with and without lipofectamine 3000). As a negative control, a scrambled ASO was used. ASOs were designed with modifications for greater stability, binding affinity, and less toxicity. For this, phosphorothioate (PS) bonds were used in all bases (indicated with an asterisk), and the inclusion of a 2′-O-Methyl (2′OMe) in the first 5 and last 5 bases of each ASO (see, Table 2).

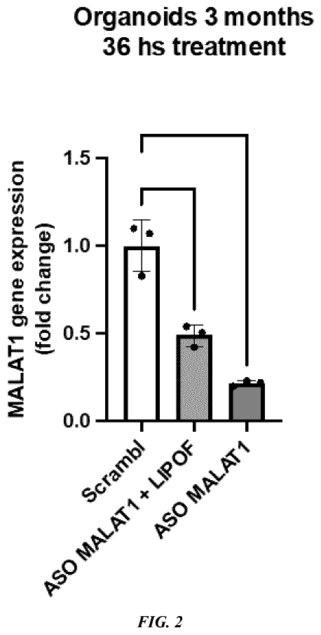

As presented in FIG. 2, the results showed better efficiency in ASO (MALAT1) treatment in the absence of lipofectamine 3000, resulting in an approximately 78% reduction in MALAT1 expression.

Based on the functionality of ASOs against MALAT1 expression 12 ASOs were designed against FOXG1-AS (SEQ ID NO:15). In some embodiments, the ASO is designed to target sequences that are conserved among human subjects or primate subjects. In addition, the secondary structure of the target mRNA transcript and the proximity of the hybridization site to functional motifs in the designated transcript, such as the 5′ CAP region or translation initiation site, can also be analyzed. Other factors such as motive search and GC content calculation; and (iv) prediction of binding energy (ΔG° 37) are also important. The ASOs were designed using these parameters, when possible, in the following regions of the lncRNA FOXG1-AS, as indicated in Table 2.

The 14 ASOS in Table 2 were tested on 3-month Wild type/control organoids under the same conditions as the ASO MALAT1 treatment. Of the 14 ASOs tested, a number of them were able to reduce FOXG1-AS expression (FIG. 3 left panel), and 3 were able to increase FOXG1 gene expression within 36 hours (FIG. 3 right panel).

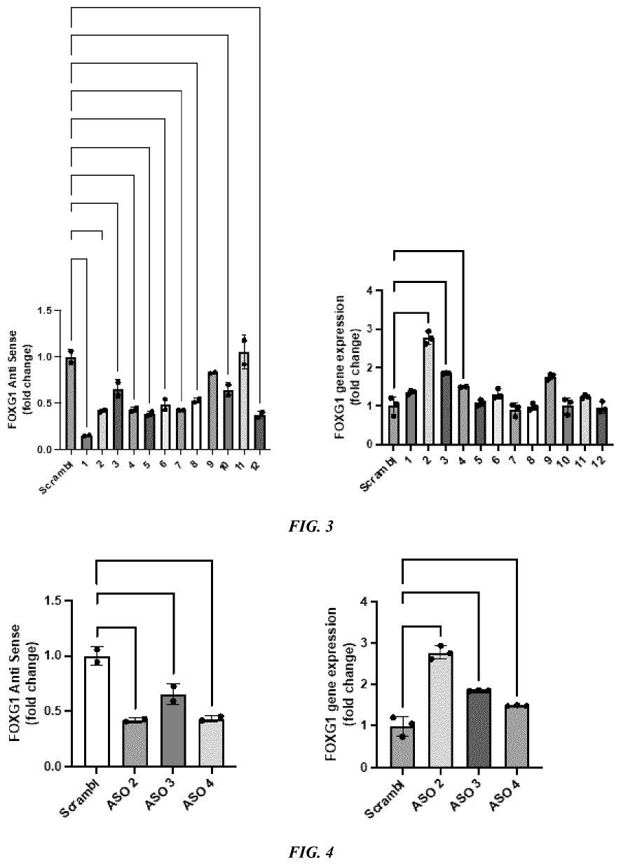

The best candidate ASOs were assigned to those that were able within 36 hours to significantly increase FOXG1 gene expression (e.g., ASO 2: 177%; ASO 3: 85%; ASO 4: 49%). The FIG. 4 shows the relative expression levels of both RNAs.

The disclosed ASOs may also have one or more modifications to improve stability, solubility, activity, cell distribution, and/or cell uptake. For example, the disclosed ASO may contain one or more sugar-modified nucleosides and/or modified internucleoside linkages, which are, for example, more resistant to nuclease attack. In some embodiments, the ASO may contain the Substitution of 5-methyl dC for dC in CpG motifs to slightly increase the Tm of the antisense oligo. In some embodiments, the ASO is an RNA oligonucleotide. In still other embodiments, the ASO contains both deoxynucleotides and ribonucleotides.

### Treatments on NPCs and BCOs

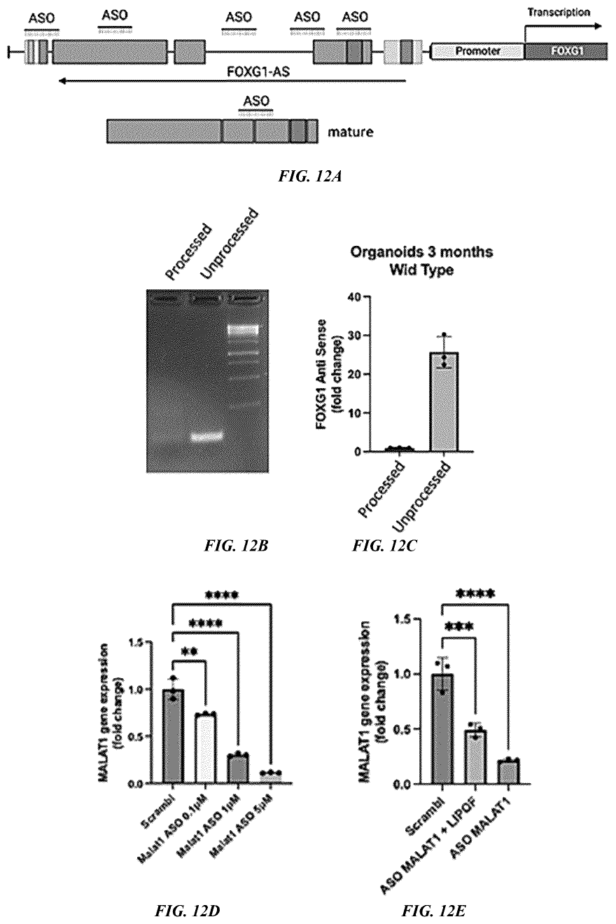

BOCs and NPCs were treated in vitro for 4 days in two doses for all compounds. For 5-AZAD, concentrations of 0.1 uM, 1 uM, and 5 uM were tested in 3-week organoids or cortical NPCs carrying the Q86fs*35 mutation. Sodium Butyrate was used in concentrations of 5 mM and 500 mM at different times between 1 week and 4 months. ASO treatments were performed between 1 week and 4 months. As a negative control, two treatments were used, a scramble sequence and PBS (Table 2). As a positive control, ASO targeting the MALAT1 gene was used (Table 2). In the ASOS penetration test, the ASO Scramble was marked with the Cy3 fluorophore for reading after 24 hours of treatment. The ASO was able to reach approximately 70% of the organoid diameter. In addition, an almost total silencing of MALAT1 (around 10%) was verified with 2 doses of 5 uM of ASO for 4 days. The 3 concentrations tested under these conditions were 0.1 uM, 1 uM and 5 uM (FIG. 12D). Furthermore, it was found that the absence of lipofectamine was more efficient in silencing MALAT1 (FIG. 12E). Thus, a protocol was established with a concentration of 5 uM for 4 days without the presence of liposomes. After treatments the organoids were washed once in PBS and frozen for extraction or fixed in 4% PFA for immunofluorescence.

### CRISPR-Mediated Correction of FOXG1 Mutants

iPSCs carrying W308X and Q86fs*35 had the mutation corrected through use of CRISPR engineering. The mutation correction and silent alteration was verified in the electropherograms by Sanger sequencing.

### BCOs Single-Cell RNA Sequencing Analysis

BCOs at 4 months were dissociated from 3 libraries: (1) 1 clone from W308X; (2) 1 clone from G224S; and (3) 1 clone from a control library. For each library, a total of 10-15 organoids were transferred to a 12-well plate containing 0.5 mL of HBSS. The solution was changed to 500 μL of dissociation solution (30 units/mL papain and 25 units/mL DNase I) and incubated at 37° C. for 30 minutes. Using a 1 mL pipettor, the organoids pipetted between 5 and 6 times at room temperature (15-25° C.) every 10 minutes. The resulting entire cell suspension was transferred to a 15 mL centrifuge tube containing 1-2 mL of 10 mg/ml ovomucoid protease inhibitor solution (10 mg/mL in HBSS) followed by centrifugation at 300×g for 5 minutes. The pelleted cells were resuspended in the HBSS, filtered in a 37 μm strainer and counted using a Chemometec automatic cell counter (viability range: 85-92%). Between 88,000-194,000 dissociated cells per sample were processed to generate single-cell libraries using AmpliDrop technology and services provided by Universal Sequencing Technology Corp. (Carlsbad, CA). Briefly, cells were first mildly fixed to preserve RNA content and then were permeabilized to enable the influx of reverse transcriptase and tagmentation reagents. After the reverse transcription and tagmentation of cDNA, cells were encapsulated in water-in-oil droplets with an electronic pipette according to Universal Sequencing Technology protocols. Tagged cDNA was amplified inside individual droplets and incorporated with unique barcodes on a thermocycler, resulting in amplified 3′ end of cDNA fragments attached with a cell-specific barcode. Illumina indexing adapters were added to barcoded cDNA to generate final single-cell libraries. Cleanup and size selection was performed using HighPrep PCR Clean-up MagBio magnetic beads (MagBio Genomics). Library quantification and sizing were made using High Sensitivity D1000 Screen Tape and the 4150 TapeStation system (Agilent Technologies). Samples were sequenced on the NextSeq instrument (Illumina) as single-end reads at approximately ten thousand mean reads per cell. Between 3K-5K cells were processed per genotype.

AmpliDrop 3′ scRNA-seq Analysis

BCL sequencing files generated from AmpliDrop libraries were demultiplexed and converted into fastq files using AmpliDrop Analysis Pipeline v1.0 (Universal Sequencing Technology Corp.). Fastq files were used as input for Cell Ranger v5.0.1 (10× Genomics) using the Single Cell 3′ v3 (—chemistry threeprime) with introns (—include-introns). Sequencing reads were aligned with STAR (v2.7.6) using the GRCh38 human reference genome. The R packages Seurat (v4.1.1) and Harmony (v0.1.0) were then used for data filtering, normalization, scaling up, dimensionality reduction, clustering, expression analysis, exploration, and visualization. Loupe Browser (v6.1.0) was used for data exploration and visualization (UMAP plots with Seurat-generated projections). Microsoft Excel (v16.65) was used for visualization (staggered and proportional plots).

Seurat objects were individually created from each sample using the CreateSeuratObject( ) function (min.cells=3 and min.features=200). Cells that had fewer or greater than 200 and 3,000 features, respectively, and contained greater than 5% of reads from mitochondrial genes were considered low quality and removed from further analysis. No software (such as DoubletFinder) was used to infer and remove doublets owing to the risk of accidentally removing transitioning cell substates. Manual examination of data was performed for unexpected co-localization of well-known cell-type-specific gene markers.

Data were normalized and scaled using the NormalizeData( ) and ScaleData( ) functions, respectively (scale factor=10,000). Data were integrated based on 2,000 highly variable genes using the FindVariableFeatures( ) function (selection.method=vst). Anchors between individual datasets were identified based on the subset of highly variable genes using the FindIntegrationAnchors( ) function (dims=1:20). Anchors were then inputted into the IntegrateData( ) function (dims=1:20) to create a batch-corrected expression matrix of all cells. Principal component analysis and UMAP dimension reduction were performed using the RunPCA( ) and RunUMAP( ) functions (npcs=30; dims=1:20). A nearest-neighbor graph was then calculated using the FindNeighbors( ) function (dims=1:20), followed by clustering using the FindClusters( ) function (resolution=0.5).

Cellular identity was determined by finding differentially expressed genes for each cluster using the FindMarkers( ) function and comparing the identified markers to known cell-type specific genes. Gene expression on UMAP plots was generated using Loupe Browser and Seurat/Harmony-corrected projections. Seurat/Harmony projections were exported using the cbind( ) function and uploaded directly into the Loupe Browser. Cell proportions were calculated with Excel from Seurat object labeling.

The iPSC lines used on this study was obtained through cellular reprogramming of skin fibroblasts from seven individuals with loss-of-function mutations in the FOXG1 gene, including 2 missense mutations, 1 nonsense mutation, and 3 frameshift mutations. Two clones were generated from each patient. In addition, two previously characterized iPSCs, derived from non-related neurotypical individuals were used as controls. All clones were karyotype.

BCOs were generated from the iPSC. All mutant iPSC clones did not grossly affect the ability to generate viable BCOs. Some mutations presented reduced-size organoids.

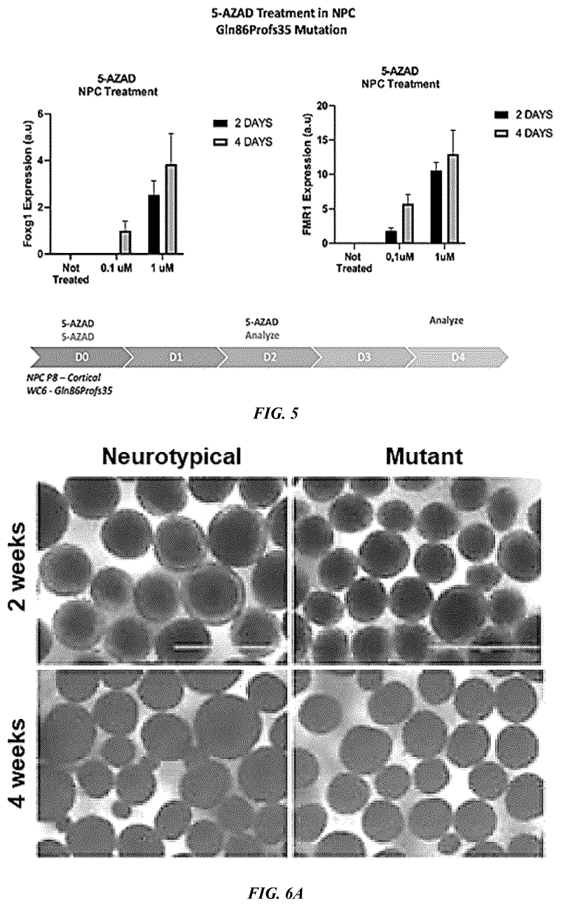

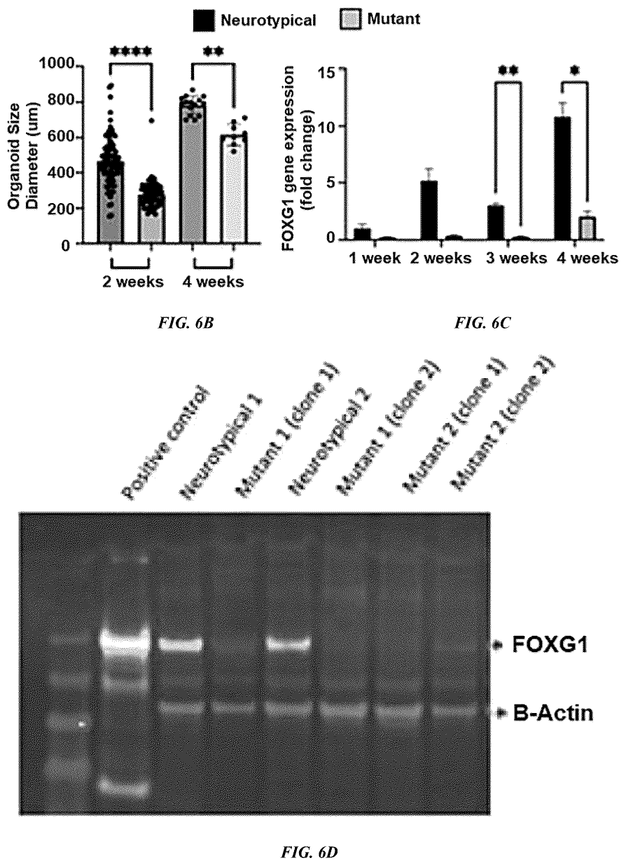

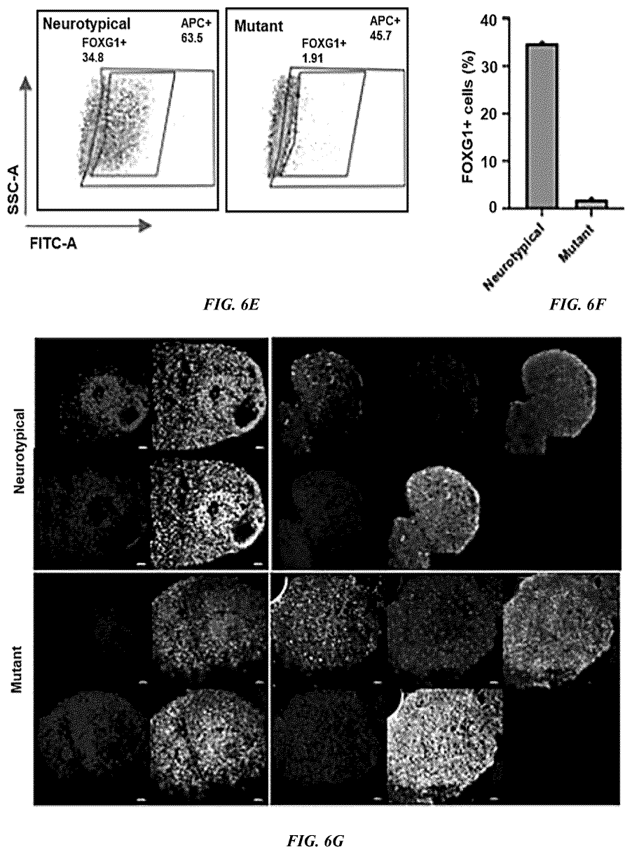

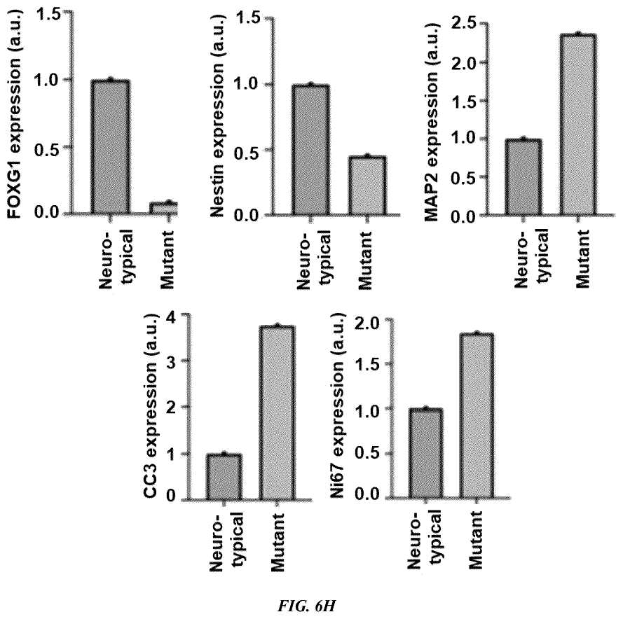

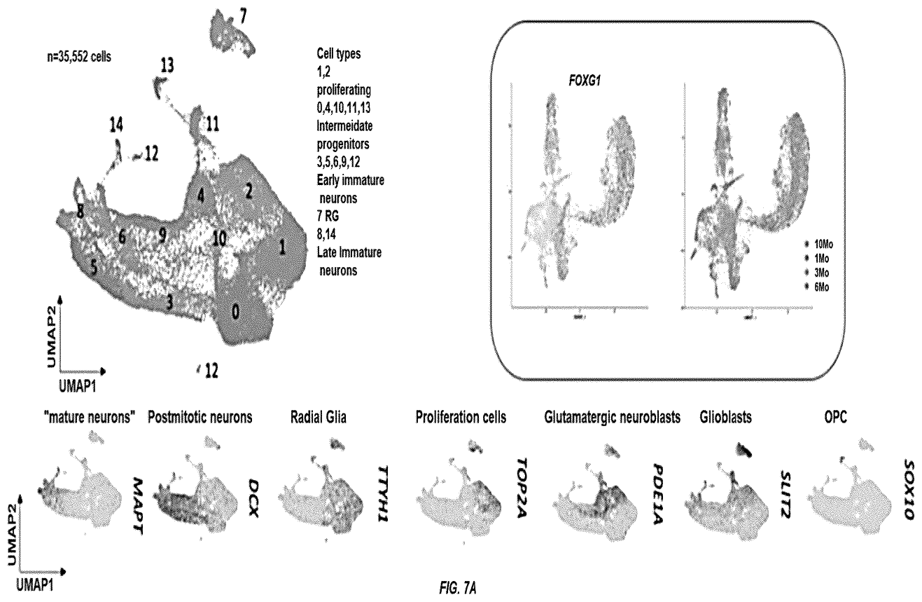

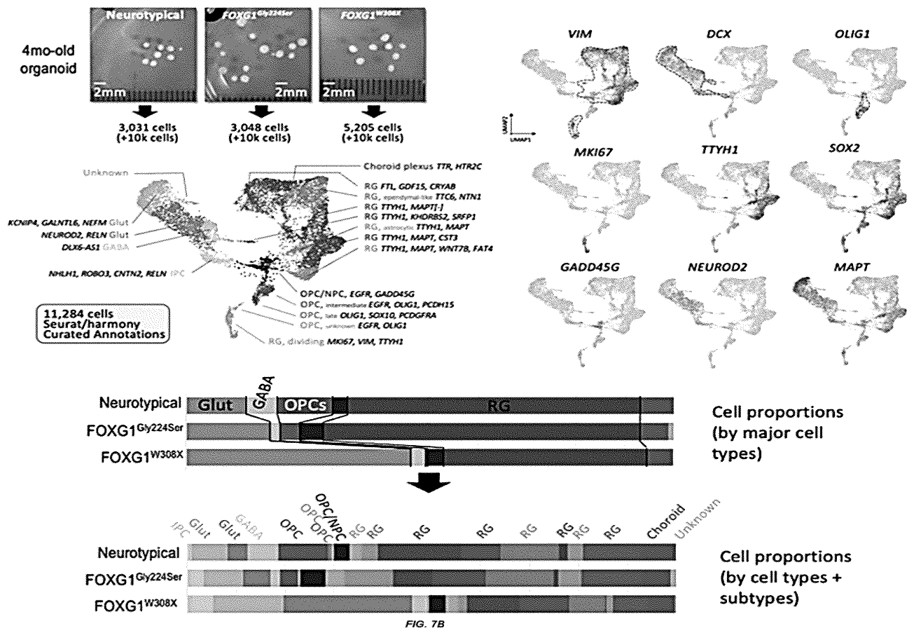

BCOs showed very low FOXG1 mRNA expression levels compared to controls during the four analyzed weeks (FIG. 6C). Moreover, protein expression was less than 5% when compared with a control in western blotting analysis. (FIG. 6D). Unexpected, this level of expression was extremely low compared to a typically observed 50% reduction by biallelic expressed genes. In view of these results, the experiment was repeated with another mutation at 1.5 months. This analysis was performed with different mutations (Q86fx*35 and p.Glu326fs*129) and from lines genetically modified by CRISPR (homozygous and heterozygous). In these experiment, both mutants had extremely low FOXG1 expression. The lines with edition in FOXG1, presented the same expression of FOXG1 between heterozygote and control, and absence of expression in homozygote. Interestingly, the heterozygous transgenic lines showed a different pattern of protein expression when compared to the analyzed mutations.

Flow cytometry and immunohistochemistry demonstrated that FOXG1 syndrome organoids contain lower FOXG1⁺ cells. Control BCOs have 34.8% of cells positive for FOXG1, while mutant have 1.91% (FIG. 6E-F).

In addition, other markers for analysis of proliferation (Ki67), apoptosis (CC3), and neuronal maturation (MAP2 and Nestin) were also verified. The results showed that the mutant at one-month presented fewer cells of FOXG1⁺ and Nestin, and interestingly more positive cells for MAP2, Ki67, and CC3 (FIG. 6G).

### Single-Cell Analysis in Mutant Organoids

To understand the alterations in cellular diversity of FOXG1-mutant BCOs single-cell RNA sequencing (scRNA-Seq) was performed. BCOs at 1 month contain mainly proliferative cells, intermediate progenitors, early immature neurons, radioglia, and late immature neurons (FIG. 7A). In addition, a significant increase in the number of cells expressing FOXG1 after 1 month of differentiation was observed (FIG. 7A).

At 4 months, BCO cell subpopulations show distinct glutamatergic and GABAergic lineages, each with neural progenitors that progress through an intermediate progenitor stage toward the generation of neurons. The analysis of the percentages of cells attributed to each subpopulation corroborated the existence of differences in cellular composition between the control and mutants BCOs. Radial glial progenitor cells (RGPs) are one of the most affected cell populations in the FOXG1 mutant BCO compared to controls. Interestingly, RGP cells were found in smaller amounts in mutant organoids (FIG. 7B). Oligodendrocyte progenitor cells (OPCs) are fewer in mutant BCOs compared to control (FIG. 7B). Furthermore, the data also show that N-GABA cells are decreased in the mutant neuronal subpopulations while N-Glut is increased when compared to controls.

### Rescue Lines for W308X by CRISPR

It is unknown whether the pathophysiology of FOXG1 can be rescued in human brain tissues. Thus, experiments were performed to carry out genetic manipulation of FOXG1 in BCOs. First, the FOXG1 mutation in one iPSCs line (W308X) was corrected using a CRISPR-based genome editing strategy. After clonal iPSC expansion, analysis of the FOXG1 levels were assessed at 1-month-old BCO. FOXG1 RNA and protein were analyzed by qPCR and western blotting, respectively. The FOXG1 expression was only partially restore in all clones analyzed.

### Altered Pathways in Mutants BCOs

Mutants BCOs and rescue lines were submitted to bulk RNA sequencing to verify transcriptomic changes that may shed light on the dysfunctional neuronal properties of FOXG1. Differential expression (DE) analysis revealed four misregulated genes such as MSX1 (p-value-1.68e-3), MSX2 (p-value: 4.96e-4), LHX5 (p-value: 4.48e-3), and ZIC1 (p-value: 2.14e-9) involved in Neural Crest Differentiation (p-adj 0.002). In addition, genes responsible for hypoplastic dendrites, fewer excitatory synapses, and reduced excitatory input as the DLX1 (p-value: 0.02). Most interactions in this pathway are proposed to regulate the transcription of core factors involved in neural crest and downstream progenitor development. Alterations in genes involved in active pathways in Fetalbrain (p-adj 0.002) such as NAV3 (p-value-1.52e-4), ISLR2 (p-value: 2.17e-5), TTC9 (p-value: 0.03), MYCN (p-value: 6.60e-3), BCL11A (p-value: 0.04), and CDK5R1 (p-value: 0.05) were also detected. Interestingly, genes such as GRIN1 (p-value: 0.01), SIN3A (p-value: 0.04), AKT1 (p-value: 0.02), REST (p-value: 0.03) and RPS6 (p-value: 1.69e-3), which are involved in the MECP2 pathway and Associated Rett Syndrome (p-adj-0.05541) were altered. Other genes involved with hindbrain development during early embryogenesis were also altered (upregulated HOXB2, HOXB3 and POLR2E and downregulated ASH2L and NCOA3), suggesting that the mutants had a less forebrain profile.

### Therapeutics Approaches to Increase FOXG1 Expression.

Given the only partial rescue of FOXG1 expression observed in the edited strains and knowing that the molecular region has predicted CpG islands and acetylation markers, tests were performed with methylation and acetylation modulating drugs in order to verify a possible positive modulation in FOXG1 expression. Furthermore, ASO targeting the non-coding RNA called FOXG1 anti-sense (FOXG1-AS) were also tested to see if they could achieve this goal.

The drug 5-AZAD, which is capable of demethylating genes, was able to increase FOXG1 expression in organoids younger than 3 weeks, but not in organoids older with postmitotic neurons. Therefore, 5-AZAD was tested in mutant Q86fs*35 NPC at different concentrations (0.1 um, 1 um, and 5 uM). The results shows that this drug was able to sharply increase the expression of FOXG1 (FIG. 5).

Experiments were performed targeting the antisense non-coding RNA (FOXG1-AS) to determine if such targeting was able to modulate FOXG1 expression. Thirteen ASOs were designed against FOXG1-AS in different regions of the no-coding RNA, including the exon splicing region (FIG. 12A). This is because both the unprocessed and processed transcripts are expressed in 3-month BCOs. It was possible to verify that the unprocessed form is expressed 25 more times than the processed form. To confirm which strand was amplified, the RNA was extracted, and the cDNA of the unprocessed and processed transcripts were sequenced. In addition, FOXG1-AS was detected in mutants and controls BOCs at 1 week, 4 weeks, and 1 and 2 months. Interestingly, NPCs do not express the FOXG1-AS, unless treated with 5-AZAD. As a positive control, an ASO previously described in the literature against MALAT1 was used, and a scrambled sequence was used as a negative control (Table 2).

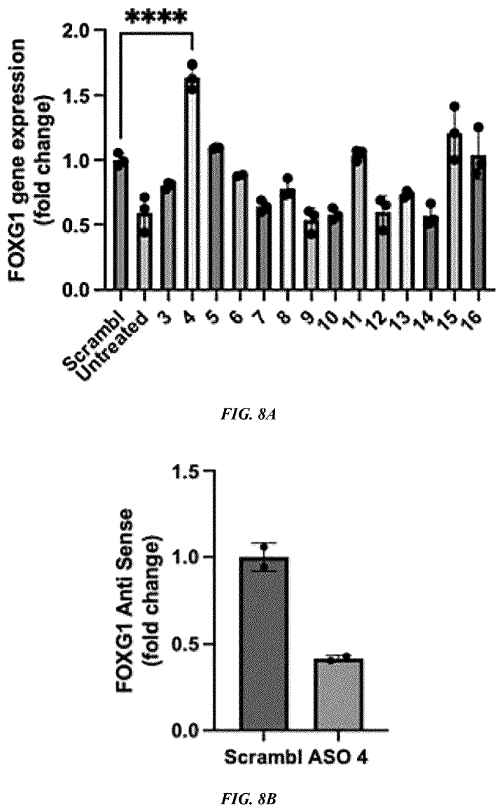

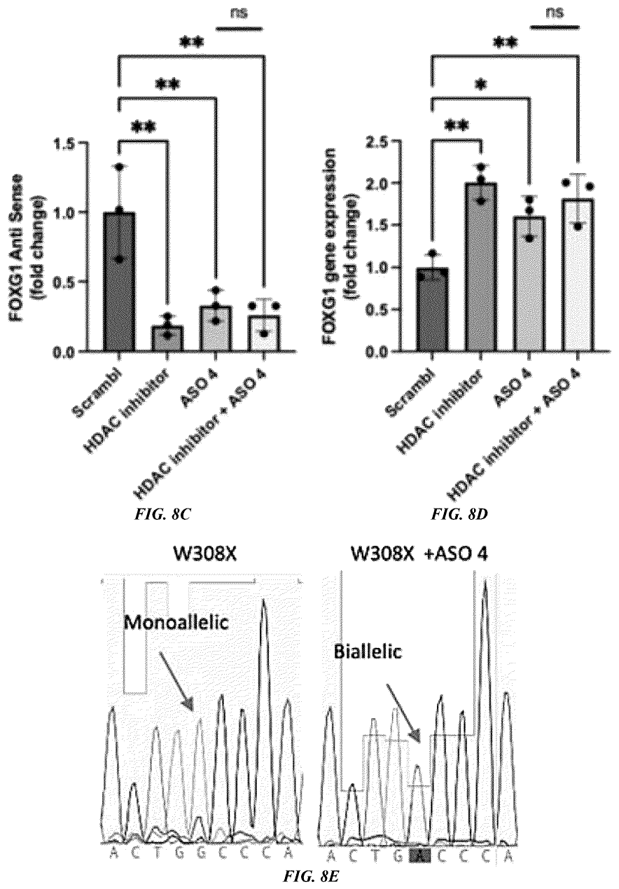

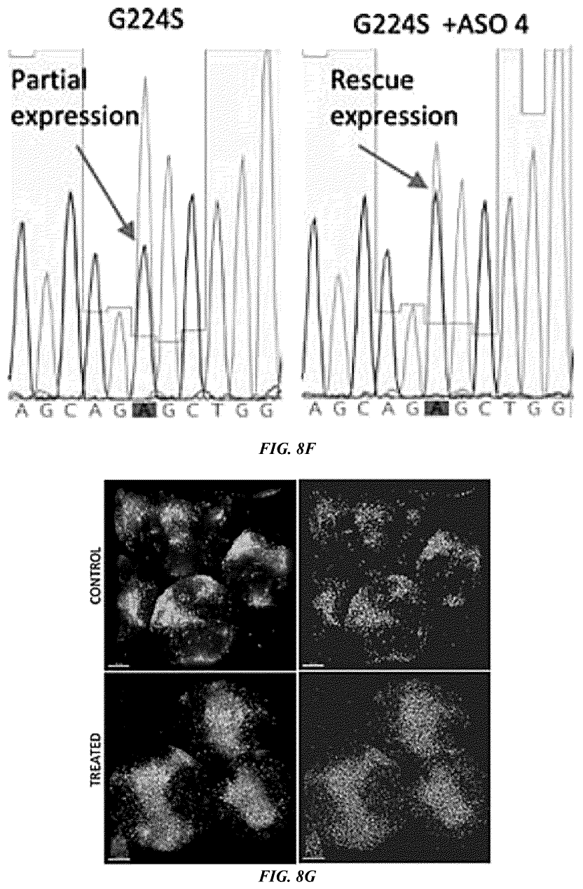

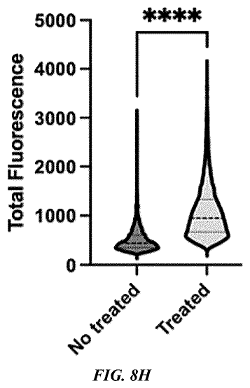

Among the 14 ASOs tested, one of them caused an increase in FOXG1 expression (FIG. 8A) and a decrease in the expression of FOXG1-AS (FIG. 8B). These data suggest that the ASO is acting directly on FOXG1-AS, leading to molecular changes in that region thereby modulating FOXG1 expression.

To determine whether FOXG1 expression in these organoids was monoallelic, the cDNA of BCOs was sequenced before and after treatment with ASOs. Expression in the W308X lineage at 1 month was occurring in only one allele and that after treatment with ASOs, both alleles were expressed (FIG. 8E). In the G224S lineage, a disproportion in the expression of the alleles was observed, which was corrected to the same levels of expression in both alleles after treatment with ASO4 (FIG. 8F).

To understand the mechanism that FOXG1-AS could be carrying out, experiments were performed to assess histone acetylation to determined if that could modulate FOXG1 expression. Treatment with sodium butyrate (an inhibitor of histone deacetylases) increase FOXG1 in BCOs (FIG. 8C). These data suggest that FOXG1 can be controlled by levels of histone acetylation.

In view of these findings, co-treatment of sodium butyrate and ASO4 were tested and neither synergy in the decrease in FOXG1-AS expression (FIG. 8C) nor in the increase in FOXG1 expression (FIG. 8D) when compared with ASO or Butyrate alone. This result suggests the possibility of some link between FOXG1-AS, acetylation profiles and modulation of FOXG1 gene.

This data was further verified by immunofluorescence using a reporter line created for FOXG1 gene. The line has a NEON sequence after the FOXG1 gene linked by P2A, thus generating two different proteins when the same promoter is activated. The results shows an increase of NEON after treatment with ASO4 (FIGS. 8G and 8H).

It will be understood that various modifications may be made without departing from the spirit and scope of this disclosure. Accordingly, other embodiments are within the scope of the following claims.

1. A single stranded antisense oligonucleotide (ASO) that suppresses the expression and/or activity of a FOXG1 antisense (FOXG1-AS) nucleic acid, wherein the ASO comprises 12 to 50 linked nucleosides.

2. (canceled)

3. The ASO of claim 1, wherein at least one internucleoside linkage is a modified internucleoside linkage.

4. The ASO of claim 3, wherein at least one modified internucleoside linkage is a phosphorothioate internucleoside linkage.

5. The ASO of claim 3, wherein each modified internucleoside linkage is a phosphorothioate internucleoside linkage.

6. The ASO of claim 1, wherein at least one internucleoside linkage is a phosphodiester internucleoside linkage.

7. The ASO of claim 6, wherein at least one internucleoside linkage is a phosphorothioate linkage and at least one internucleoside linkage is a phosphodiester linkage.

8. The ASO of claim 1, wherein at least one nucleoside comprises a modified nucleobase.

9. The ASO of claim 8, wherein the modified nucleobase is a 5-methylcytosine.

10. The ASO of claim 1, wherein at least one nucleoside of the ASO comprises a modified sugar moiety.

11. The ASO of claim 10, wherein the at least one modified sugar moiety is a bicyclic sugar moiety.

12. The ASO of claim 11, wherein the bicyclic sugar moiety comprises a 4′-CH(R)-0-2′ bridge wherein R is, independently, H, C₁₋₁₂ alkyl, or a protecting group.

13-14. (canceled)

15. The ASO of claim 10, wherein the modified sugar moiety comprises a 2′-O-methoxyethyl group.

16. The ASO of claim 1, where the ASO is a gapmer.

17. The ASO of claim 16, wherein the ASO comprises:
a gap segment consisting of 8 to 12 linked deoxynucleosides;
a 5′ wing segment consisting of 3 to 5 linked nucleosides; and
a 3′ wing segment consisting of 3 to 5 linked nucleosides;
wherein the gap segment is positioned between the 5′ wing segment and the 3′ wing segment and wherein a nucleoside of each wing segment comprises a modified sugar moiety.

18. The ASO of claim 17, wherein each nucleoside of each wing segment comprises a modified sugar moiety.

19. The ASO of claim 17, wherein the nucleosides making up each wing segment comprises at least two different modified sugar moieties.

20. (canceled)

21. The ASO of claim 18, wherein the modified sugar moiety comprises a 2′-O-methoxyethyl group.

22. The ASO of claim 1, wherein the ASO has a nucleobase sequence that comprises at least 15 consecutive nucleobases of any of the nucleobase sequences of SEQ ID NOs: 1-14.

23-24. (canceled)

25. The ASO of claim 1, wherein the ASO is a gapmer consisting of a 5′ wing segment, a central gap segment, and a 3′ wing segment, wherein:
the 5′ wing segment consists of 3-5 modified nucleosides,
the central gap segment consists of 8-12 nucleosides, and the 3′ wing segment consists of 3-5 modified nucleosides;
wherein a modified nucleoside of each wing segment comprises a modified sugar moiety; and
wherein the ASO has the nucleobase sequence of any one of SEQ ID NOs: 1-14.

26. (canceled)

27. The ASO of claim 25, wherein each modified nucleoside of each wing segment comprises a modified sugar moiety.

28. The ASO of claim 27, wherein the modified nucleosides making up each wing segment comprises at least two different modified sugar moieties.

29. (canceled)

30. The ASO of claim 27, wherein the modified sugar moiety comprises a 2′-O-methoxyethyl group.

31. The ASO of claim 1, wherein the FOXG1-AS nucleic acid has a sequence selected from the group consisting of SEQ ID NO:15, 16, 17, 18 and 19 or a sequence that is at least 80% identical to SEQ ID NO:15, 16, 17, 18 or 19.

32. A pharmaceutical composition comprising the ASO of claim 1, and a pharmaceutically acceptable carrier, diluent and/or excipient.

33-34. (canceled)

35. A method of treating a subject having a neurological or neurodegenerative disease in need of treatment thereof, comprising:
administering a therapeutically effective amount of the pharmaceutical composition of claim 32.

36. A method of increasing the expression of a FOXG1 in a cell, comprising contacting the cell with a composition comprising an antisense oligonucleotide (ASO) complementary to a target nucleic acid, wherein the target nucleic acid has a sequence selected from SEQ ID NO:15, 16, 17, 18, and 19 or a sequence that is at least 80% identical to SEQ ID NO:15, 16, 17, 18 or 19.

37. The method of claim 36, wherein the cell is located in a brain of a subject.

38. (canceled)

39. The method of claim 37, wherein the subject comprises a mutant FOXG1 gene.

40. The method of claim 37, wherein the subject has FOXG1 syndrome.

41. The method of claim 36, wherein the FOXG1 nucleic acid is a ribonucleic acid (RNA).

42. The method of claim 36, wherein the ASO has 18 to 20 linked nucleosides.

43. The method of claim 36, wherein at least one internucleoside linkage of the ASO is a modified internucleoside linkage.

44. The method of claim 43, wherein at least one modified internucleoside linkage is a phosphorothioate internucleoside linkage.

45. The method of claim 43, wherein each modified internucleoside linkage is a phosphorothioate internucleoside linkage.

46. The method of claim 36, wherein at least one internucleoside linkage of the ASO is a phosphodiester internucleoside linkage.

47. The method of claim 46, wherein at least one internucleoside linkage of the ASO is a phosphorothioate linkage and at least one internucleoside linkage of the ASO is a phosphodiester linkage.

48. The method of claim 36, wherein at least one nucleoside of the ASO comprises a modified nucleobase.

49. The method of claim 48, wherein the modified nucleobase is a 5-methylcytosine.

50. The method of claim 36, wherein at least one nucleoside of the ASO comprises a modified sugar moiety.

51. The method of claim 50, wherein the at least one modified sugar moiety is a bicyclic sugar moiety.

52. The method of claim 51, wherein the bicyclic sugar moiety comprises a 4′-CH(R)-0-2′ bridge wherein R is, independently, H, C₁₋₁₂ alkyl, or a protecting group.

53-54. (canceled)

55. The method of claim 50, wherein the modified sugar moiety comprises a 2′-O-methoxyethyl group.

56. The method of claim 36, wherein the ASO is a gapmer.

57. The method of claim 56, wherein the ASO comprises:
a gap segment consisting of 8 to 12 linked deoxynucleosides;
a 5′ wing segment consisting of 3 to 5 linked nucleosides; and
a 3′ wing segment consisting of 3 to 5 linked nucleosides;
wherein the gap segment is positioned between the 5′ wing segment and the 3′ wing segment and wherein a nucleoside of each wing segment comprises a modified sugar moiety.

58. The method of claim 57, wherein each nucleoside of each wing segment comprises a modified sugar moiety.

59. The method of claim 57, wherein the nucleosides making up each wing segment comprises at least two different modified sugar moieties.

60. (canceled)

61. The method of claim 58, wherein the modified sugar moiety comprises a 2′-O-methoxyethyl group.

62. The method of claim 36, wherein the ASO has a nucleobase sequence that comprises at least 15 consecutive nucleobases of any of the nucleobase sequences of SEQ ID NOs: 1-14.

63-64. (canceled)

65. The method of claim 36, wherein the ASO is a gapmer consisting of a 5′ wing segment, a central gap segment, and a 3′ wing segment, wherein:
the 5′ wing segment consists of 3-5 modified nucleosides,
the central gap segment consists of 8-12 nucleosides, and the 3′ wing segment consists of 3-5 modified nucleosides;
wherein a modified nucleoside of each wing segment comprises a modified sugar moiety; and
wherein the ASO has the nucleobase sequence of any one of SEQ ID NOs: 1-14.

66. (canceled)

67. The method of claim 65, wherein each modified nucleoside of each wing segment comprises a modified sugar moiety.

68. The method of claim 67, wherein the modified nucleosides making up each wing segment comprises at least two different modified sugar moieties.

69. (canceled)

70. The method of claim 67, wherein the modified sugar moiety comprises a 2′-O-methoxyethyl group.

71. A method of treating or ameliorating a FOXG1 syndrome in a subject having, or at risk of having, the FOXG1 syndrome, comprising administering to the subject an antisense oligonucleotide of claim 1, wherein the antisense oligonucleotide comprises a sequence complementary to a sequence that is at least 80%, 90%, 92%, 95%, 97%, 98%, 99% or 100% identical to SEQ ID NO:15, 16, 17, 18, or 19.


SEQUENCE LISTING (29 sequences)

SEQ ID NO: 1 - DNA, 20 bp, synthetic construct
  source 1..20: mol_type = other DNA
attacatttattctgctccc

SEQ ID NO: 2 - DNA, 20 bp, synthetic construct
  source 1..20: mol_type = other DNA
gacttaaatgcagggaagag

SEQ ID NO: 3 - DNA, 20 bp, synthetic construct
  source 1..20: mol_type = other DNA
tactccaattacacaaaccc

SEQ ID NO: 4 - DNA, 23 bp, synthetic construct
  source 1..23: mol_type = other DNA
caattcattagaaatgggttccc

SEQ ID NO: 5 - DNA, 20 bp, synthetic construct
  source 1..20: mol_type = other DNA
tgccccagctattatctgag

SEQ ID NO: 6 - DNA, 20 bp, synthetic construct
  source 1..20: mol_type = other DNA
cagttgtgagttattagctg

SEQ ID NO: 7 - DNA, 20 bp, synthetic construct
  source 1..20: mol_type = other DNA
ctgtctttaagttatggcca

SEQ ID NO: 8 - DNA, 21 bp, synthetic construct
  source 1..21: mol_type = other DNA
atcctcattgaggcttgggaa

SEQ ID NO: 9 - DNA, 21 bp, synthetic construct
  source 1..21: mol_type = other DNA
aaaatgtggtatgtttcgtgc

SEQ ID NO: 10 - DNA, 19 bp, synthetic construct
  source 1..19: mol_type = other DNA
agtgtggctttatataccc

SEQ ID NO: 11 - DNA, 19 bp, synthetic construct
  source 1..19: mol_type = other DNA
taagaagggtctaaggagg

SEQ ID NO: 12 - DNA, 20 bp, synthetic construct
  source 1..20: mol_type = other DNA
aacatttactgcatgccagt

SEQ ID NO: 13 - DNA, 19 bp, synthetic construct
  source 1..19: mol_type = other DNA
tcacagaacattttaaaga

SEQ ID NO: 14 - DNA, 20 bp, synthetic construct
  source 1..20: mol_type = other DNA
tcaggctgtgagtaagagag

SEQ ID NO: 15 - DNA, 41877 bp, Homo sapiens
  source 1..41877: mol_type = genomic DNA
ccatgtccagcatcacccagtcgtcggggggaaacggcggcggcggcgcgggagcgggacagcggcgcggcgggcggcggcgaccggtgggtgccgaacggggcgcggggggcgcgggcggcagcggcggcactgagcgctccggcagcagcgcagttggaccgcaggctcgggcgctggcaagtcatgtagcaaaagcagcaaccacccctcaggaattagaaaaaaaaaaaaaaaggaaaagaaaaaagaaagaaagaaaaaagaaacaaccaccgccccgggggtgaagctccctcgctcccaactgtacttcttggtctccccccccaccccccgctcccccccccgctgcttcctcctcctcctcctcctctcgcagcagcagtcacagcagcagcagcagcagcccaaagggggggagagccgggcggcgggcgggcgcgtcccggagcggccgcggcgaggctggggagacagcgatcgaggcggctatagccacaattaattttccgagctacaggcgcacactagggctgtaaaaaaatttttggtttaacctttcctaccgctgggccaatcagagctggcggcgtgcgggagcgtctctctcgccgtcgaccaatcggaggggccgggcccacccacccccgccccgcccgccggctgccgccccctcgggcgctcgcctccccctctccgcgggcctcggaccccgaggccgcgcggagccgcgtagggatacgccggcgggagcacgcggtgccgcagcccttccctgcgcccccgccaccccctccaccccgccggctccgctccaccgaaattcccctcctagggccaatagctcacatttccggcttcctcctcattgtactgggtcattttcgtttaagcgtcaatatttgtccttagtgattaaagtttgtatccaattttatttttgaaagcgggggtggtggtggtagtgggaagagtgggagtgcagggagaggatgcgagagggcaggaaatggaggaggtagaggcgaggcactacttttccgagtcggccttgcaggccagtgtctggcatttctgagccagcaggaaaattgtgccctttgtaggtaatagtctagcgcagctttggataaggggggagataaggcccaccgctggcgtgtgtgcgggggtgtcctttacttcgggggaggagggtgtgggagagctttgggagcgatgagatgaggaggaggaagtgggcagaggagcgcggggccaaacaggggttacttactcgattcctgaggcctgggcggcggcgagctccgggctccggcaccgcggggaaaccgggactggggctccgggtgctgctgcggcggccggggccgctcagtagtgggcgcctctgatggccttccgaaatgagaggtgcccagagagaaaacagtaccaaacaatctcgaaataaatcaggtaacacatgctatatacgtgtgtctcttccttttccttctctcctgctccagagcgtcgctcacgcgcggggtgactcgatgtcgttttctcctcgccgctccagcctcagcttctaatccgaattggaagcgggtagctgcgagcgatcaccctggaaatattttcgttttgcttgttgacattggagggaaaataagggtgggcgtctatttctttccggttttttttttaagccttcctattttcactccttttttttcctttccgtttctcaaatgcttttattttgtgcgggcctcattcctgaaataacagtatactgaagccagctcttgccaaccctgagtgctgctggaaagcaccggggagccttgggaacgtggatctcacagtcccagcttcttgggctttggaactgggaaggttcggttggaggttgaagtgaatggggaaattgcgaatcgggggctttaaaaggatcccgtggccaggggcagggctaaggcccagcatttcccagggattggaatttagtggggagcacattgatcaaaggctgtttcatcccacatgggcacgaaacataccacattttacggaggatgtagccccagagaagagggaggtggagaagcataaatgaaaggcaccttttccttcctctagaaggtaaataattataaaatgtaagcatttcttcacttggataaactttcattaagatatctggcagatttggtatttaagaaaacgaaggaaggaaaattggtgcttatgcttgatcctaaagttaaattgtaacataaagaatgcggttgatgaatactcaggataatagggggttattccatcctccccacccccagtcttaagaaagagaaagagagaaaaggttgggggggggggaggaaactggagagaataatagataattacaaatagctagtgaattttaaatatgcatctattaggaatgccacaaaggagctttgaatattggactaacctcaaatgtccttccactgtatgtatgtatctttacctatgtctacatatcttctcactttaaaacaaactgtatttacatgtggaaaaattcaagggttccaacaatcagtgttgatagtgacatggtcccaaacatttatttccaagctctggatttctcgcagggccgggtgagctaatgggctttgcctaccggctggagagatttgtgggccttccccttcctactcacttttcctcctcccactcttctcccctcttcaggcccgttcttcctacttttcctcaactctccttcctccctcctccctccttctcccttagctccccccgccccttccccccaccctccattcctctgtctctccattcctccacctactcctcccaatttctcctcttacagccccccttctcaccaccacttataatggcacacagggctccctctgaggtggacggcccggtacgttttcgtagtcggtctagcgtgaaatgattcggtgtaaccggattcttgggaaaggacaggttacgtaaacatttactgatgtggagtaactatactatttgtactctgatatgcaggaaggaaagtgttgattaacacagaagaaaaaaaaaacgaaattagctgattgcatgctcataagtgcaagctgtcattgaactgcttttaaaatagacacagctgggtgtttgtgttgaaaaatttcttgtgtttttgctaatactgaaataagcatgattttgtataccaaacattttcagatcattaaaatttgtaatctgtgctggttttaaacacaaagaaaagaaacagaatcatttcgatgtaaaatttgctgatattatattaactcaatcaggtctgtacaaattgtgcattgtgcctcctaaaatctgtttgatcaatttaatacacaaagtgccaaacgtgggtattgtttgttaacattggacattttattcaaatccgagctcagcagcaaactggaggagctttccttcaatacaacaccagctctgactgatgaatttttaatgttttaaggtgtttcttaggtcaaatctgagcgtgcattgttttgtaatcctgtataaaattttagcaggtggaaataagtttttcttctgtttactttcaaatagcaaaaactggggtttgctaatgagcactgtaataatctgattttatatgctaggagctcatattgactgggaaaatgtttttcattcaaattaaggctatgttatatagtttaaggattgcatgaatgcaagtctctcaaaataggatattcttatgggcataaatagtatatagtttaatgtaatatttaaaaatctattcttcctaatgacaaaagttatttgtaatctggcaagaagtgagttttaattccttttaataatttcaattgcactttatgtcgagatggtgatatatatatatactcacacacatatatatgtgtgtgtgtgtatatatgtatatatatatagcttgttcaacaaaacatgcactgtttactcagcaccccgtgtttgtctcagcaatagcttttttaaagaactgctactatttgaaatggagggggaggggggtcctggacagagtattgtgcaagttgaaagtctctggatggggctttgtatatcctaccagccaatttgggtgcaaattggatttgaaggcctgcctctgtccacctcgggcctcttccctaagcactggaagcccaatcttcagccccactcagtcaccgtgattaccttcagaagacagagggaattcagttgtctcagacctgaaacaaattctctatcattctaatttttttctcatgatggatctaaccttgatctctcacacagacatctacaaatgtgtactattaatgaataaaagcagaagtcaaagtaaatgtaattttctggtatatatcacagtaaacttctctcttgctctgtctgcagtcccccagctctcccaaagaggtcaggccccgtaaagagtgagcagttccaatggaagaattggttaggaaatgaattagattattccttctggagttttaattaataaccaaggctacacagcatacttcatccgagtagggagcagaacatttcatttttccatttggcggtgttatttaagtattctttagggcctctttcctgttttcagtcagtgatccgcctcttttcttcttgctagctgctggttaaaggaaggtgggccccatttacctaaaatccactctgctctccattccctggacagggtgtccttggagtggcacccagcgccaagctctagctaaagttctcttcctgcagatgcacagagaatttctgaccttgtgacgtttgggagtgaggagatcccatacagaggcatccaggtatttccagagatcctgtggcaggtgaggtctgccctcgctggagccaactcgtctataatatcttcctaacagcaggtaatggtgctaacatttagtgattagaagttactctttagcgtgttgtatcctcgttctgtgggttttggttacaggttatacgattaggtttgcgagaacagtagtgaactaaacttctcagtaagaggtcttcacaccagggaagtgttacttttctgcagcagccctgcctcgtcactcattagggagctgcaggttgtcagtggctgctctcagcgcgcgcacgcgcaccctcaacgtccccgggggcgcgcacattcgcgcgtgtgtgtacacacacacacatacacacacacacacacacacagcactaaaactaaattaagaagccgatgattaagacaagcgtctactaggcctgtcatttcagcatccttccatcataacattgtggcaccggcaattttgcttgagaacctcgagtaaaaaaatctatgggagagcctcgtctatgagagtggaaatgtttagcttgtgtaagctagggagccactttaaaagggagaaagtccaaggcgaagaagaggagcaggaggaggaagaggacaaggatgaggagaaggccttgacagagacccctgggactctccaggcaagtctctgtgacgctgccaggtgttccaggctctctgaagccaagtacgtacgcagccaaaagaaaagtcccgtgatcttcagcaaaatgtgcatgggagaagctttgtcccgacattagccaagggagagccaggcgtggaaagatccatgacgaaataccaccagagggccaagggactttagtctctggaaaaacttctcagtcgaatactctgtacatctcttgtaaacattgtaaacataatgtccactacccttttacttgtttcgcgttctttgccagtgccctccagagggtgagtgatttgctgaaggacttgagtccctgatagtgtgagggcggacctgttccatttttagacctgatccagttgtgagaaatggtcccacacagcctggggaagtgcccctgagaggccgattcctatttttcctctagttacccaaggaaatggtgtagtcagtctttcaaaggagataggggaagctccacaaacaaccataacacagcacaacaaaacaaaacaaagagctgaaggggtgttggtgaactagaatgaaggaggctagagctcaggttcttagttttaacccccagctctatcccataaggaagaaagtgagacttggcaaaagccagcccacaaacaatattacctcggattttccatcagtggggcaccaaaacaggaaaaaagaaaataactatatatatctatatatatatatatatctatatatagatatatctatatatcaagctgaaagaaaaacttacagggctaaaaatgctgaactcttaagagctggaactcctaactatcccatcattgttcttacttagttggagcttcaatgtgatttctagtagaagcctcaatcttcttctggaagtgttacaggaccttttagagaaggggtgtgtgtgtgtgtatgtgggggtgggtggcgggtgaaccaagacttctcttttgaagcacacctccaaatggtcccccaaattccagttgtgtgagaaaagatggaaaggtctgactccttcgcaatttttagccggtcaccttcgaactagtcaccgctcaatatttaaatgtacacggaaaacattgaacagttcaaggtccgtatatgagattaataagtatcttataggaaatgcttgcacccactaaaggtctgaaggaatgagaggagagaaaacttcatgagtggggtaagcaagccaggaagcacgcagagaacccttcaccccaaagtctccctttgcgtggggaaagtagtatctactaatcagatctatctgcttctttcaagaaacctgcgtggacaaaccaatgggcaagtttttactttgtgctcatcttacacccacctggccctcactctccggagtcggttccagtaggtcttcctaccccaaccctcgctcctcccccgcgccccttctccaacacgaggccatggaaaatctgttggaggagcgtttctcaagaggtccagcttttctccctcccctgccttgcctcttgaggctgtaactgcgagggttcccttcgaggagcttcgcggagctggggctcccaggtctgcgcgccgctgctccctgcgcatacccgcgagcttgcccgacgctcctttgttgcagacgcttgccgaacgcaatcgagccagaggccttgattctaggcgtcatctctctagatgtccccccttaaagttgccttttcagcctaggtatctctttttttggcacataagaccggatacacaccaaatcaattttgggctaagaggtcatctgccacggtaaacgcggggatatagaaaacagttaaaggtttcaagtctgtattgctataaagcacatttcagagttaaaagactggggtgcatgcaggtgacgttaattcgaatattcaattacatgtaaggggtattgaacccagtgggggataacagtaatacccgtatttctctctcgctctttggaaggagtcgcctgcggggaggagaggagaagacagactaagctgcgcgtagagcggcatcaggagcaagttaccgttagcatgtgtaaacaaaacaactcgactcctctgtgtcagaatcaacaacatcaaaggtgtctgtggggggtcacttttatttttaaaacaatcctagattcttgttcggcaattttagtgtcttccttctgggtttgtcctaaggcacgtaggagagaaagtaatgttcgagaaaaaagctgggtcggcaagtttggctccggagcctatgaactatgaatcaccgcggtgaagaggttttcttttttctttcttttttttttttctcaccctcttcttttctcccggagcgtgaacttgttagataagatagatgaggctacaaggaaacagaaacgctgaaaaagcccagctcctttggagagcagcctgtggggctggcaggcggctgccgcgcgccctcccccagctgaggggcaccggggtagctccggagctgtcccaacttgggcctcctgggccccccgcggggccagacatcacgctcagatcgctgccttggccatcttaccttttggccatagaataaacaccccgaaagacgagagaaaggcctctatttgagctgagtgtctgctctcccgctcgaaacacaaaacagggcccgcaggttttcctagaggaagtcccaatcacaaaacagtcggacagtgggagacgctagagaattctggaattacacatgaaacattattttcctaattaattaaaaataattctcttttcctatttatatgcatacacacgctactgattacacgtccctatttacttgaaaaaatgcttggagtatctttagtacacaatgctcattctttgtccccttgaacatttacagctgataatgtggctggttgggatcaattagcactggattttgccccaggattgctccccagggcggacaagtggagccaactcatctccagcgacttttacttcgctcacgtgcatatccacgccagggctgcagaagcatttcaaaatccgcagccttggtttcacttgattttgttgttgttattgataaatattgggtcatacacaagccacttgtcagagcctttggctccacctcgcagctgcagctcctagcgggacgtttgagtcttgtcctaggaactggaaggatttactgggaaaagaattacacattctctgctccttcagttttggagatggtcccttaagatcatatcatatcgtctgtctgcttgctgtcacatttatttcttgattgttctgatatgtgaacaatgggaaggtgtgtgcatcttttccccctatctttttaaaacaattattgaaatggtttaaaagtgggggaggggagaaagtattgatcccagttctttccgtatatttggaaccaaaagaacataggcaaatagataattgttttaattgtcggggtggggggtgggttggtaggtaggacctttggggtttgtgtaattggagtaataaatacagtttatcttttagctccgccgtatatattctttcccttgatacatattgcacatgaaatgaacccatggtctacttgtcttagcatgtttgaagccccagacacccaacagtatcattacaggaaaaagacagagcccccaaagactgcatgcaggcaggaaggtgactgcctttggggccaatggggaatttatcttgacgttgaacttttctgggaagtgcacccactcgctcaggctcccttgctgcgcagagttcaggtcttccccatgctgaagagcagggaggaggtgtggaaaaaaacagctgatttttgacatttcaaagggactctttctatttatcagtgttaccgaagtgtacgttactggagaaacaacaaaacaagcatagagctgcaattttcaagaaaataggatccccaaatataaaaataaaaaagtagcagataagatgtataattcaatcaaattacacaaaattgcttgaaaatctagaacagtagcgatccattatgcctctgttttattagtgacaagtacaattactctaaaataaagaacactggtgaatatataattggctgtaatcatgttagtgtttgaataaaaatgacgggaacatttaacactttgacaacaaaatgttcagaaataacttatttattcaaacagtttttattttccaccaagtgaaggagaaacacagtttgcttaggaaaatcaatgacttttccctgcaataaataaatatccaaaagggggaaaaaacagccccacacctctatattattactgttattatttttaaccagcaagaagcttaatcctgtgtttgaagtcagcgctcatctagcggttgtttggtgccaaagtccgccacctagaggaagagctctgcagcccagaaccccttttcccgcggtgacctcgggaatcaaagttaatattcctttgcaaattgctctccaaactaactccccctccctttcctcctcacgtcccctttcgccctacaacatcgtcacaatattttcttagtaattgctctgcacaattgacagaagcgccgcgtctccggcagcgctgtgctctcccaggaaagcgtgagggcgcgttggggaagcaggtcagggaatatttggttctggagagacaatccactttcatccacaatggtctagagcagcgtgacttgggaacttataaagtgacggctgatggaggcatcgccacattccaatgcattttttaaccccgggctttcttagaaaaaacaactgggatccccaggtaccgcggggctattcccgacttttccccagcgcccccactccccaccccaaacgcgaagatctgagatctcggctttatttaatcatctactcagttattggagctcagtgggtccttttattctcctaggtgggttgcttgaagaactgagttacaaaggtattaaagaggaggccgtgtttgcaggggtccccctgtggtcagggaaaccgactcaggagaacccccatgtttttacactgagctgagcctccgagtttcaagaaacttgggcagtgtgcgagagaatttggggttgtactcgcaagcaccaggagtctttttgattttagtgttggtggatcctatcggctcctcacacagaaaagccaacctcactcttcaatctcgagctcatcctctcaagtaattattggggaagggagatgctaaacgagggagcatggggctctgggaagaaatttaacaacagcattcgagcgctcaaattctccttaacaccagtgctgcagattctgctgcgatccccgctctggccctccaggaatgtctccccgccccactcctccaaagttttgttttaattgctttagggcacttagaatattgttcaagttgaggatttcctagaaaggcgaggaagatagagttgatttagttttgaaaaaaaaaagttattaatcataagacttcagttgggtttcatttcttctaattttccttcctgtcccctccttccctccttccctctttccctccttccctctttccctccttccctccttccttccttccttccttctttccctgctttttcttcccttttctctttcctctctctccttctccctttccctctctctctcactatctctatgtgcgtctaactgtatctcatctacctccctaaatgtgagagaaatgaagcctcgtgaaggtgatggaggtgaccattttcattctgacttccagaacctccttacttgtcaattcttagtctcaatgctggagtttgaaagacatctcggaggtgattcttctccctcccagagacgacctcagaaccatggtctaagaagctatttttctgactgtgaaattattttttaaggcagctgaaataaatatatatttttaaaataagcctctgaaactgatgccccggtgcatgcttcctctatggattgaggcgctccctaaattcactccacccaaccccctaggagcttaagtcctcagaaatgttttgttttgttttatgcaaaactagcgcttccactctctactcccaggtctatcattcctctcccgcattatattaacggcgatgttagccttcccctacgtgtttaggctgctaatgccggccacttaacgcaacaaaaatgctcatctgtggagattttcccaaggctagctgcaatgttggctcctttttcttttcttttcagggcacgtccattcctccctgcccccttcttcttttcttgagatggggactggaaaaggggtccaacaactctaagcgagtgtgtgaagtttcccctctttctcttgcctgccaatccgtcaagcggggtggtgaatttttggttttctctagagagcaggactatggaagcttcagcgtagattggttgcctctcttagttaacttgccttcttcttcttcttttttttttcccccagtcatgactttcctttctcctcaccttagtcggtgaaagcagttggggattgccctcacagtccttctgcacccttcctagccccaggcttctgaagaaaactccactggctttcttttgtattgacagggaagctgggttggagatctcctctgtattggattgtgtgtgggtgttgtgtatgcttgtttagttttaataactttatgagggagagtctcctgagcttgttgacatttttcttctatggggagattttctggagcacggaatcaccttaatttagataatgaccgtctattaggcttcttaccctttcaagtaaatttagcctgggaaaatcatctgccctcatgattaatacatacctagtgtccaaggagaagaccatcaggggcaatgcaaactgaaactgacttaaagggtaataaatattacccagtgggttttgacttaaaaagatgaatatcgtcattgttcacagctggaacacgtttacttcttaaaagctggtgaagtgtagacacgagaactgttttcccgactctggctctagcactcttttcctatggatccttaggcaagttacttcatctttccagttttttcttaatctggaaaataagtgacctgcaatcactaatatataaaatatcattcattcatcaaattcccaacaacactaccactaaaatataactttaaaaaccataagaatctttggaaaaaatttttggggaggtggggctgggaagcaggaagaaaatctcattctaacagcagcagcgacatctagtgcccataatgctcaatagtagcagtgaaggtgtgggattgtttactgagtttttggaggggagagccatggaggcttttctaataaatcttgactgtctgtcgggcacacaatagtcttttgataataaaaaaaattctaagctttgattatggtaataagacatagagagacaacggtttttacctcaaatctgcacatagggcaattaaagtttgttatgagtattcttccaatgctttaaattccatgtcaaggaactagttctaaaaagtaattttattttgaggggggttgcaatttatttacctaattttccaaattatcactaaatatgtttctacacaaatacttaacaaaattgaaaaaaatttatttttgatattttcttttatctgaactaaccactttgattggagataattatctagtgaatagcagggaacatactaaaaataacaccatgttttaagtaatcacatatatgccctgtctatattaccttggctgaaaatattttataattcaaagaaatggaattcctatgtctatatggattctgtttcttcttcttacagcaaagattaaatgaactatctgggcacaggaaaaatcttagcaatggtagactgcagagccccaaacttttcagaataacaatttcggcatttgcttggcatttaagatgttagagagagagagagagtagacctcttctcattccctgtgactgtaagctggccatgtgcgtgggtgtagtttgtgcatgcacttgctgaagtgtgtgtttaatttcctaatagcaaagcagctgatgactactggtatatggattcatgttttgtaactttaagggaaaccaattttgtttattctatttttcaccaatatttcctagattttactataaaatttggtcactggcatgctatctagctagttctagaatagtaagataaatatacaagttgttaacacctaggggaatttgaggtagtcacaacacttctcttgaaccttagttttcttatgcacaaaatcagaaagttgaatctgctatcccttataattcaaaagatttttaagtcaataaatcatcaaccttcatttgagaagactctcttgtggtgtcttcattgttatttgcaactcttctctatcactcctgagttctctagttaggcatctcatcagaaatgagaattgatgagccagccctccatcttctctatcttcctgtcttgttctttccctttttctgcttttaaaaataacaaaaatctcagacactctagtcaccatttactaactcattggcatgcactcagctctcccttgctatccatccagaaaaatcataatgtgagataggaccaaactgaacacaaagctgaggcagctgtattcaagttttctaacatgttcccatatcctcatggtgatgattaattattattattattattattattattaggaaccagagccaaagtattctggctgcagatagcccacatttttctctctgcttctgtgtccctgtgaataaatttcttcaggaggatgcagtaaaatgtctccaaattcaatctgactaggaacatcattacacacttttagcaataagagtgttcacagaaggcaaatatcactccgtgatgcaaatatgctcagcatttaatatagacctcagggaagctgaagacacacacacatacccatgcacacacacacacatgcatgcgtgtgtgtgttgtatataaagtgtgtgtgtgtatatacatatacacacatatgtgtataaatatatgtatatggagtatcatatctacattgacctgggttgatagaaacaaacaagtttgttacagggaaaattctaaatatatcaaaggtttctttcttagatgcctttttgttgttccctagaaaacttcttagttcctactacttggacttttatttaaaattccttatgctgtacgaagttcataagacatgttttattctgcagatttttcacagacctcataaacacaggtattattctattttagaagtgaggaggctgacccagacagactaaattacttgcttaagatcactccactaggaagacacagaaaaggagtgtgaatcctggctttggacaggccgcaacttacacttggtcatttttagggttggagccttcaaactgggaaacaaggggattgactgaggctgttcattaaacttcaccaggattccaccgcatatatttgagaaatctcaacttttaaagaattttgggacccctagtctgtgtttgctactcctcattcatgtatgattttctcctcttaacactaacaattttatattctttctacaggaaggataatctttggaaaaaaggttgtactctaaacatttttgttaattatcttgggaactaagattttagagttggagggaatctaagacattttgtgattcagcagctctggagtaagatgattgtccgaatcaatggctcctgagaactgtgtgaaatgggagtccaaattattgtcagttctctggtgttgcttcctacccatctccctggatttttggctttatgcattaatacatttggttacttcattcttgctggccaccctgtggactagttttccaagcggctgcattttcagcagcacactgtggacttagccaagcactttaagctgagagctgttaagccttcctggatctattttctctcaagtagctctctgctgccctcaactggtgttcagccaccagagctagctagccttcagcatttacacgaatccaccgtaccctggcttcaagtcagatatgggagcatcattaaccacactgtttatggcttttatagcatggaaaaatagaacccaggcccattcacgtccacttatgcctcaggttctcactttctacaagtttgcagatgggccatgaaggagaatatctgattttcacttatgtgtgctggagtatttggaagggcttacatgaaaatgtaaactttctctttaattatcaaattgagatacacttcagctgatagaccaaatttttgaacatgttgcagttagtcttaagaaatacgaaacattttgtctttgatgtaaacaagatatagagaatcatatgaaatccatgaattacttgtaaactttctcatttgttcaataaatacatagcgtacacatactgtacgctagacattattattggtgctaagaatatagcattggataatggaggcaaaaacctcttctgcctttattaagctaacttcccagttaaggaaaatagacattaaacaagtaaataggtaaaatacattttaagggaatgtaacatttccatatctatttcctaccccaagtactgtagcgattgcatgtttttgttttgttttgttttgttttgtttgagacggagtctcactctgtcgcccaggctggagtgcagtagcgcgatctcggctcactgcaagctccgcctcccgggttcacgccattctcccgcttcagcctcccgagtagctgggactacaggcgcccgccaccacgcccggctgattttttgtatttttagtagagacgggatttcaccgtgttagccaggatggtctcgatttcctgacctcgtgatccaccccctcggcctcccaaagtgctgggattgcaggcgtgagccacagcgcccggccgcgattccatgtttttaagaacatttgtagtaaagttaaagaggcataactagagtttatacagggaccatgcatgctatataaatattggatgcgttatttattaatgaattcataaatttcttgtgaatatagagtagatatatgaaagtgtttgatgcagatgagtgtttgaacctttcagctgaagagacgttttgtcttttgtttctataccaatctctcctgttgaattgccttgtggttggaatatcactttgtcaccccctgtaatagctgccactaaagtccttgctattgctgctattgacgatggtctctggttattatctttacttttctctttgtttttattctgttctcttactatgtcttttgctttcttctcagataaactctgatagataccactgaccaaatagaataagttttccctttcttatgtggtggaactaccagtcataactagggaggtttaatcacctgattttcttttcactaccaggtctatttgaaatgggtatttgtcctaggtaaaatgcagctgatttggaagaaaccaaaatattaatcaaataataaccaagtggtagtggcaattacagatacacagcttctaagtaatatgtttgtatttaaataaataaaatagtccaaattctgaaaaaaaaaccctataatataaaactgagtttgagttgctgggtgagaagcttcatccttgccgtgaaaaatttttctgtgtggaactttccagttttgtttttatgatgcagacgtcttctgctaggcagacatgacaccatctcagtgaaccatgtaggtcagcacagggttaaaaatatgacgtacctatccttatatgaattagaaatgtttatatttattaacacatttcttgaaatatctttaatttttgtttacttctgattttatatatcacagacttgctgtaaaaaatttctagtatattataaagaagaaaataaacataaatcatctattaaagtaaaacctatattctaaataatttggatcatcttgtgggtatattttgtgttcagccttttgttcattattgttatactgtgagcatttccacataactatttttaaaacctatagttacaaatatctatattaacctatggataatgataaacaatttaaccactgtccattaaccattgcttcacatttaggtggtttctaatttttcattattaaaatgacttgagatatataactttgcacataagctttttctccaactttgatcatttataatagattttttaagggcaaggtttttttttttttttggtggggagggatcacatagtatgaatttgtaataaacatttaccatatactggtgaattgtatttctttaaaatttcattactgtggctgggcttggtggctcacgcctgtaatcccagcactttgggaggccgaggcaggtgtgtcatctggggtcaggagttcaagaccagcctggccaatgcagcaaaaccccaaccctactaaaaatacaaaaattagccaggtgtagggttgcatgcctgtaatcccagctactcaggaggctgaggcaggaggatcccttgaacccaggagacagagggtgcagtgacctgatatagtgccactgcactccagcctgggcaacagagcgagactctggcaacaacaacaacaacaacaacaacaacaacaacaacagcaacaaagattgtattagtgtacatttgcagtgatctataaaagtgactgttttggccgggcacggtggctcacacctgtaatcccagcactttgggaggccgaggcgggtggatcacgaggtcaggagatcgagaccatcctggctaacacagtgaaaacccgtctctactaaaaatagaaaaaattagcctggagtggcggaacgcgcctgtagtcccagctactcggtaggcttaggcaggagaatcacttgaacccgggaggtggaggttgcggtgagccaagattgcaccactgcactccagcctgggcaacagagtgagactctgtctcaaaaaaaaaaaaaaaaaagtatctgtcttaatgaaccctcaccaaattttaaattttcttttgccagtttaaaaggagaaatataatacctataactagctttaatgtacttttctttatgtaccagcaagattgaaagaagtttctacaaatatttattacattttaaatttcatcttttatgatctttctgttgtcttttgtgcattgcttatttgaatttttgtgctatttttattaattttaagggcttttctagataaaaatattaaccgtttgtcatctggctcaaatatcttttatagtttttattggtctttttagcctatttacaatatttagttatacacaaatgtaagttttatgcatattcagatattgaaaatgtaatgagcttgaaacttaaaacctgggtttgaatctatcactttgtgtctatgtgaacttgagtaaattattaaacctctgtaccttatttttatttgatataagatggggttaataatgctaattctctcataatgttttctgaggataaaatgcattaaatttataaggaatatagtttgaagtgtaccaggcaaatggtaagcccaaaattaagattgaacatttttattattgtttacagtattcaaatttacaaatgcaaaatgatttttattactttaatattagtattttacatatctaaaatgagttaaatactaagtaaagttttattaactaaatatcaaatatagctaagaattttgtatacctagttttttaaattatataaatgattaacttgagtattctgaggaaaaatactattatgtcttattcatgtacttaatactagttttattcattcaagagatatttgttgaacttttactacatgccagaaactgtagttgaacaagatagcgaacaaatggatgaaaacctttgcttttctgcaattgacggagaaaccataacaaaatacgttagtatcatgaaaaatataatagagaagaataaaggagataagaagtgcaaaaaaagtgcttgcaatttcaaatatgttagccaggggagatcactgagtgtattagttttctgttggtgcctaacaaattactacacatttaaggcttaaaacaacataaatttatttttttttccagtttctgtgggtcagatcaagagtctagacacaaactacctgggtcctttgctcaaagacttacaaaactgaaatgaaggtattgactgtgctctggttctcatctggaattcaggttgttgatggaatttagatccttgtggttgtaggactgtggcctctgttttcttgctgcctctcaactaaggtcactctcagctcctataggacagatccatccatcataatctgaataatcacccttttgattagctaaagaaaggagtgttggtgatcaattgcatcaaatgttgctgataggccaagtaaaatgagaagagaagattgactggatttagcaatacgaaggtcattggtaatcatgaggagaacagatttgttagaatggtggaggtaacagcctcttgggtgatgattaaaaatgaatgggaagaaaaatttgagatagtgtatagacagtttattgagcaggtttgtggaaagggaagcaagagaagaaacattagctagagaggaggtggagccaagagagggtttttttaatgggagaaaagtatgtttaattgctgatggaaatgatacagtagagaggtaaaagtaaaatctgatggcataggagagaaggggtagaattgctggaggaagtgagatgggatcaagtgaccaagtgagatggttggacttaggtgggagtactgataatcatctttcattccagggggacagcaaagtacatggattctgtaggttgtgggtattctcttctgattacttcagttttctcaatggactaggaagcgagctaaaactgagcatgataatggaagtgcttgaaatatgagtaggaaagattaggcatgaaaacgccatcaagaagagtgggagagtaaaagaccaggcaaatttaatatgattgccagacagtgttatggacatacatgaaatgcatgctcatgaatgtaaatgaaaccagtttacatgctgtgtgttttcctaagtcatgttagtttcagggccttcagaactagcaagacagaaagtaatatgtaaccagggttgtgattcagacaaatgactctgaacaagacaggtaagggaattggggatgcaaaaaggagtcactgcaatgattggtcatggaatttaaggtaaaaaggggggaaaaaggagagaagggggagagaaaagtgaaaagcataaattgaagacctaccaagcactatggtaaattattagaataaagtaatacaagcttttctttaaaactggctttctgatgactctaaggctgtgctgtatagagaggtcaagtgatcctgagctttagctcctcatgagctgcaggtaactcccatcacaatgacttattttgacaaactcaaagcactttcatagctcctatcccttggtacacttatgaagaaagcaagggcaggaactaatatctttgtttttatatagatgtaacatctttattttatatctagatatgcataaagagaggatccctggacacagtttttaatttaatttaatttaatttaattttattttattttattttattttattttatttttgagatggagtcttgctccgtcgccaggctggagtgcagtggcatgatctcagctcactgcaatctccacctcctgggttcaagtgattctcctgcctcagcctcctgagtagctgggactacaggcgcatgccaccacacccagctaatttttgtatttttagtagagatggggtttcaccatgttggccaggatggtcttgatctcttgtactgtgatccacccacctcagcctcccaaagtgctgggattacaggcgtgagccactgcgcccggctgacacagttctataatatacagaggtgaaattattgctaactcaaatcactgtttctctgtgtgactgctttcagcttccttagttctagtcctcagcaactcatgctgccctatggttgttccacattccccactgcaatcttcttctcaggattttagtcagcagccatgcactgtaattttctcttttcccaggcttccttcattgaaaacttcctgtgaagttctcagttctattgagtaacccattaaccactttggtaaacgaattccaacttgacctaagttcttaatcttgttatctgtcttagagtggtagttttcagtatgctcttccctcaacccacagtaagaaacacacatcatgacctaggccacaaatacatgtatctacatacacacatacacagctgaaacaaaaaattttccaaaatacccttattacttgtgatacactctgatagtttctattctattatattctatttaaaaatttactggttgagactcactaagctatttaccaactactaatggttgatggactgcagtttgacaataaaaggggcttttgatgcttataatgcaggtaggctataatcatggctcataacacatttaagtattttacaaaataactcctaattgagacctgataggatatgtgtattttagtagctgaaaagagctggaagttagcagttagtagaaagactatagaacttcaaaagacccaaatcctatacttcattctaatacttaataactttgacattggtttcttattctaccctctctcatgttctgtctatctaaaatggagttcaaaacacctgctacgtgtaaccctgaaatttattctcaagagcaagtgaactgtaagtaatggaggtttataaatagcaaatgtaagactctctaattttatttctggattgggtttatgttggaactctgaaagtcaggcaaaccctccaaagataagaatattccctgtcaccaactgctttgattgatcttaagttgccatatattattcaaatatgttgaattatttgaatattatattgtttcttatgtattcaaataaaatttatcatttatcagaaaaatattttaaagttaaaaaattatctacttgatatgatatggtgtataaagctttttatgcactttaacagaaaatgagtcatggttttttattaggttttgagataaatccacacaccaacctggaaggcatagttctaatacagctgctgtacattttcttcacagcttatttgtaatgtctttagtcattgttaggtctgttagtcagaagtttcacatctataatgagactcacttgtggtcatcatttttagtaattatttaaaaaaatgttgacatacaattttgtgtgttgaaattcagttctattcacagatttctccttctgttcttctgcaaatcaacatggataatctcaaaataacagtgcctctctacactctaaggatttaggttgtgtcatcatattaatattttgcccttttactatttccattaattaatttagccctttaacttttactgatcaatgtggaccaaattttaattgcaacattgctatgaaataataccaaattaaagctgatagtgtcatgctgtgagctagtgagagatattaacatttttgaacatgataagttgatttaaaaccagatggacttgtaaccaacttcagaatagtgttcaaaatctagttgtgaagaattttgaatattacattaatggttcaagtttggggtgtctgataaaatgctggtggcttggaaacatgtagctttacattatctctatcccattatactgttattagaaagttaaaacatgcaatgatgtaaagaaggaattcagtagtgtatctgatgaatatctattaattcccataacggatttaaatattaactttaatatagactgtgcaatatggttgattatttactcattaactaaacatttaatcattatctactctgtacaagatactacacgaggtgatgaagacagagtgttgaataaaatttagttcctactccaatgggactcacagtctggtggaggagatgaacacacagtcatatgtatagcataaaatcagtgccacactggaggtatgtactgggtgggttctatggaagtacagaagaaagccacttaagtcttcagattgtttcatggaagaaatgtcattaaatctctgtcttcaaaaattatcttaaaaatttatcgatattgtacatgaaaacataataataaataatttagaagagcttacgataaaaataacagaactgtttctcttcaacagctgtttggattcccggctgtgaactcttttaaatacctttttaaaattttctttcttttttttttttttttttgagacagggtctcactctatcatccagcttggagtgcagtggtgcgatcacagctcactgcagcctcgacctcatggctcaactgatcctcctacctcagcctcctgattagctggcaccacaggtgcagcccaccatacctggctgattaaaaaaaaattgttgagatagggttttgccatgttgcccaggctagagttttaaatacttcgtttgcatttctttttgtgattccttcatgatactaattatattatacttattatgggtggtttagagttatttatcatttcattacttccacaggttacttttgtgataatagtaataccctaacacattatgagatattatcatccccactctttcatccaccttttacctcccttagttcccaccatttattagctaacctagttttattttattaagactattataatttgtactttgaaattaaacagcaaccaaatctctcaccctttctccatgaattaagtctggaaatttacatttttatgactatgctgagtcatgcattgtgctatgacagaatacattctcttttctcactatcaattgcttaaaaccatgccagagatttaatttttttccatttgtatcgtagccttttacacaactttcttcctttattttctttaggttagccatcaacttttctttctttctgtctctgtcttttttggcttcactatattagcacattattgttttaaagaggtaacatgtctttattgcgtttttcacacattctcccagtgattctcaaatggggaggtcttttctcacatgccttcctcctgacaccatcaatccttcctgccatacaggaatatcctgcattgctttcctgggtgtgacccacacttttttcctgtctctcctcttctttcttctctttattttcttcttttttttcttgtttcgtttcttatttttctttattttctttaatttctttatttattttctttaaaaggtatgtggtaaatacataccatattaccaaatgcttttattctggtgtgtatgtgtgtgtgtatgtgtgtgtatctgtatgcgtgtgattgaactgtgtatatcatttttaagtcaaaaatattttctcaaaaactgtaaggttttggtctattttcttagcattcagtgttggtgataagtttaaactaatcctttctctctctctcttttttttaatagaacctgggtctcactctgtcccctaggctggaatgcagtggtgtgatcatggcccactgcagccttgaactcctggacccaagcaatcctcccatctcagcctcccaagtagctggaactacaggtgtaagccacacctggataactcatttatttttagataacctgtttttaatattggaagcttttatgattatctctgggtaggggtctttacattccacttggcatttttcactttaaattctcatgtctttcaacttggaagaatttgattctatcagttctctgttgatttcttctactccattttctgtattcacgttgtttttgggtttctattactcagatatggggcgtccataccacttctataggtcttttatctgttccctcatgctttctgactctgcatttctgctttacactgtaacagagttctctattttattttctagatctttactgttaattccgtttagtagtttgttctgcattttccagttgttagaaatctgttattattcctcattttttgaaaacttctttcctacattcttcttagttacgtattaattctatttttgtgcatttatgttcttattttagtgctgtctcaggagagataagatgtatcgctaagacctagtatgaatttgagtcagaactataagcagcaacaggttgttttagggttaaagtaacaaactagaaacagtagggccacaatttgcttttttttttttttttttttttgatggagtctcaaatctcgctctgtcactcaggctggagtacagtggtgctatctcagctcattgcaacctctgcctcctgggttcaagcgattctcctgcttagccccccagggtagctaggagtacaggcacacgctaccatgaccagctaatttttaatttttggatttttggatttttagtagagatggggtttcaccatgttgtccaggctggtcttgaactcatgtcctcaagtgatctaccctccttggtctcccaaactgttgggattacaggcgtgagccactgtgtctagcctgctgagataatttctaggtgtcatatcatggagggtcttgtagatacactctactaaggacactggacttttatcttagaggcactgaatgttctaattgtgaaaatagtatgatcttgtaaagaaggtatatttaaagactagaggccaggaggacagttagaagcctgttgaaagaatttatgttagtgataaagatctgatccaaagcagaaagatcagagagaagatgaggcaaaaataatagagatactacagaataaatttccaaaattgagtaaaaatatgaagacaggaaatgctaagaacgccaaataggataaaggcagagaaaaccacatgtagtcacatcacaataaaacttccaaaaaccaaagatgaaaaaaagcaaccttaaaaacagccaacagataatcaaacaatatatattaccttcaaaggagcaattaaaggcaaaataagagtgttacctttcttctcaaaattagtaatgaaatccagaaaataatagaatgataatcctaaaaaagctaaaataaaatacgggacagcttataattaaagcctagtctctctctacttttgcctaatgtactatgtccctgttcattaaggcaatatttcgtcttaatgaaatgtccctgttcattaagaaaatataagaaaatactttattctcttataattttgtggagctgatgttcataaaatgcatcatttaaatagttaattttgtaagatgatgggcttggcagagtaactgagatttaaatccttgctgtttaatctgttctaattggttaagggtaatgcccaatttagaagcaaaaagtaaaattgcagatttgtttttaactcccttcagttctgaagtttaatagtttcaaaaacatctcctttattatacttaccctaagaaagttgactcagattaattgtggcaatatttgataaacataataatgccaatattagatgaaatgaaaataattgtgaatagaaggctgtgtgtaataaggggattataaagacgtaggttcagagcagacctttatttatttacttaataggtgagtactggttctgttgcctagtagagtgcagtggctattcacaggcatgaccatggcgcactacagtcgcactcccaaatactgggactacagatgggcaccaccacacccagctgtagagtggatcttttttttctttttttttcttttttttttttttccccgagacggattctcactctgtcgcccacgctggagtgcagtggcgcgatctccactcactgccagctccgtcttccgggttcacgccattctcctgcctcagccttgtgagtagctgggactacaggcgcccgccaccacgcccagctaacattttgcatttttagtagagatggggtttcaccgtgttagccaggatggtctcgatcttctgacctcgtgatcctcctgccttggcctcccaaagtactgggattacaggtgtgagctaccgcgcccggccaagtggatctttgtaagaactgtgggatagcaaatatagaattagggctgtgcaattttatgtccagatagactattatgaaaataaaattgacctccatgagctctggagttagatttcctgtgtaaaaactccagctcttcaatttaccagctgtgtgaccttggacaagtttcttaatgaatctgcatttcactttctgcatctgtaaaaccttttcaagattgtggcataaatttgtaagcatacttcaaatgttaagcctttattataataatgaaaatttggcaaaacaaagctgatgcattcatagtaattgtgtaatgtatttattaatattttattggattctggtatttggcttaggcaaaggaagtatctgaaaatatttacgttttatgtaatgttaaaagaatgaaaagaaaaacaaaaaaaattatcaatatatgctaagaattttgttgacagtccacatatataattttcttaaccctaaaatgaatttataatgtaggtgttattttttgttattttacagacgggaaaactggcttagagatgttaaatatcctggattattgtcagtttaacaaaataatttcggctatcaaagatcaagtgggataaatttcagaggaagaaactgagctccttgcgtaagtcctagaaaaaacataaaaggtggttaactgcctccatataaagctgattttttaaaaagctctcattttaggtttaggggtacatatatggagttgttatacagataaattgtgtgtcatgggggtttggtgtacagattatttcatcaccctggtaataagcataggacccgataggtagttttctgaccttcttcctcctctcacctcaccctcaagtaggcactggtgtctgctgttctcttctttgtgtccatgtatactcaatgtttagctaattctagcctgtaaatttaatcacaaatctcctgttaacaatgctgtgttgcttccttctttggctatttcaactatctgctgatttcttttctttttactactatttcatttgcagtggagccaaccaatcctaacaaaattgaatagtagataaattataccatttcatctgaagaaatacctgtctcatttttactttttctctcactctatcctctactaaaaactgttttcagataaacttctgaaaccaatcatttataactggatataaattcaaattatttttacttctcagaatattatctcaagctaacttgaattgggtaataaaagacattttactctattaaagaaaagctaggcatttcctgaaatatcaattagtttggaagcaattcacctatatgtcatgtgaaccctaaaataaaataaaaaaatctagactggttatgtatattttcctgttgaaaaagatctagatgattttttaaaatgctacatgttactttctaataaatattttttaaaaagttggtcaccagactgcccctaagcaataagagatttccaaatttttatataaagtggaattatgaaacctaattatgtacaactgctgttctaccatgtttgggctacattggtaggaatacctgttagctgtgcattgtcaaagattgcttatacttggagatatgaatgtttggccactcctagttggagccaggcctatattgatcaaggataatgaaaggaagtactgatgatggtatttctttgacagtttcagttaacaggaagtattagtctgaattagggtgtaacaaagggaacaatgattcagtaagttaaagaagatagaactggttttgtcctttacataacaatccaaattgaatggttcaattctgctccatgaagttattcaggaattaaggttctttccattgattgcagtattatcctctaggactgtcatcttctgcatggcagagacagggtcagctgccattccagctggtgataaggggaaagataacatggaagcatacacacggtcctaagacttaggtccataagggacacctatcacattaaattccatggggaaaagaagtccactatgaaaaggaggctggaatattccatttattcctgcatgcaggaataaaaggagagtatcttttggtagacaactaatactctccactttatggtttgtgctgtttgttgaccctattgaagtttcctgatgatgtgcagtcttttctttgtgaaaaggataaagattgatacggtttctgaatggacccttaaattggacgaataataccacccttatttttctgtagactgtgttttacacttgcctatactgtgaaaaataataatcttttattggataagagtctctccattcttattttcttcaacttatttggtctcgggaaaataggaccaatattaaagctgtcttgtgatttacctatgccttgtgaatctgtttctagtcagtttcacttagaaggaaaagggccagattctacctctaattctgcaaggaaaaatacattcttagtagatcagaatacattttaatctacccttttcctgaattagattaaaaaagctttgcttactaaatagcttacaaagtgtcttcttttgaaataacctgtacagataactttgacagggaatttactagggaaattaaacaaagctgcctttattgttaatgaataattcttcaactacaatttaatattagatatactatatttaaaatgaagagagtaatgttcattcttgtaggatagtgattattttctaattttctattaggaccaaacttatgttttgatgtaatgttgggttttgtatgtcacaagtctccagcagatccagaaaggcccttttcacctgttactatgctcacttactagctgatgtctaagtttctctgctagaaattatttatcttggcttaaggctgtaccttgctaaaacccaattatctgtgaaaaaattaaccttttaaactttataatcattttatattttgttctggctgaatttgttgttgagatttttgaaaatgagctagaagaatccttgaggtggctgtgagagggtccactgactatggaaatgaacacaaaaagatttttgtttaaaagatgaatttaatttgtgaattttctctccattgtaggtcactttgagttctggctgtcttacagaggaaaaaatagagctgatcatgaccattataaaaacgtattatgtgcaaaagatgtacatcattccctgaatattttggtttttacttcaaattagaaaattactgttgttgatttaaataaaggacagcatcaacaaaatcatcatgaattggaaatagtgaaatttaatttttataaatctgtgctaaacattttaatggaaatatatttttaatatatattttaatgaaaaaattttaaaatatatattttaatatatattttaaaaatatattttaatgaaaaatgtagtggtgacaaattcaaacaagtaaaataggatttaagttatatgtgttaataaggatattgatctactgttgatattctgaagctattagcaatcaaatgttaccattaattttcagcccttgagctactacttagttttctactcaccagttctaccttttaaaaaattgcaaacaacatggctgtatgatgagggaaaagctttacccctacttggacaaaaaataggccctgcttgtaagattagttgaatgttcaggtccttgtaagttgtaaagaatgaagtttaaaaatttgtgttaggggccgggagtggtggctcactcctgtaatcccagcactttgggaggccgaggtgggaggatcacgaggtcaagagatcaagaccttcctggtcaacatgctgaaaccccgtctctactaaaaatacaaaaattatctgggcgtggtggcatgcacctgtagtcccagctactcggtaggctgaggcaggagaattgcttcaacccgggaggcagaagttgcagtgagctgagatcacgccactgcactccagcctgggtgacagagtgagactctatcccccccaaaaaaagaaaatgtattaggtacatttttttatactttaaatttgatttaagacattatcttgactaccaataactctactgctgtgaatccttgaaatttaatgtttagatttagatctaatagataactacattgtcaactctgtaaatgcttggccattttattaatgttgctattttctagtgcttgcatatagtagacagtaaataagtattgagtggatgtccaataactctttgtcattttaatttgtaagtaattattaattctttttctttaggatacaggctaaacaagagtcaaaggtaagccaaatgagctggagaacagaggagagattagacaagggcaagtgtgtataaggtggtaaaagagtaccctgagataaatgtaaatggtgtgcaatagaggtttaaaggatagagcgcttgtacacagtttggcaattcaggaatgcctccagtaggatatggcatttgaggttgaacctcgcagggtttgtgtggctgagtggtgaattgtgaggatagtcatggcagagagcacattctataaagattagagcaaattcagatgttttccatctctgaccttgtatggttattttacagcacttgtgtcacacactgttttaattattttgtactgtttatacctctcatgccatccactaaattataaacttcttgagggctatgtgttagtaagtcttgtattttctgctaagtctatgagtgctctgtatacattaatatttttaaaaaattactttatatctttcaagttatcaaagattttatagttttcctaattacgttgaatctagtgattaacaaatgaaatagctccttgaataggctgcacaatcaagcatttctaaaacagcagtttagagctaaggcgctggagttagaattgccatctttaaatactggtttagctgttgactaacagatcttgacaatttgttaaccacagtggactcagttttcttatctataaaaagggataatattcatacatacctaatagagctctgtggtagaatatagattgctataaatactttgctactcttccttttgaaagatggagtctaatattcatccctttgagtctgggctggcatttgtgacactcttaaccagtaaacaataatggaagtgatattgtgagacctttgaggctagatcatatgaagctctgaagcttttgctgatctctttttgaatgctgctcttaaaaatctttctgttggagcctttagcctccacataaggagtttgattactccgaacctttagccatcatataagaagtttgactactctgagactcatgagacatgggatctcatgctggagatccacgcgtggattctctggttaacattactagctgagctaaactttttggccattccaggcaaggcatgagacacgtgtgttaagcctcctggtatcctttataacagcccatccaccaggtgaatagcatctggtgatctcagctgacttcatatgtagaactagtatcacctattataacccaactcaatttcttgacccactttcattaaccataaaataatggttgttgttttaagacaccaagttttgaagtagtttattacatggtaaaaaataactggaacaggctattatgaagattaaatcatttgctgtatataaaacagcacagcgctgggcacatattatgtattctataaaagttattgctattatgatattattacaccataaccacaattataaagtctgcatttgtgtgtgtctgtgtatgtcatgtttgtgtatgttatttgcaagtattgattaaatggacttgaaatgaaagccaataatatgctgcactttcatataagagaagctttaagtcatttttttaaagatagggtcttgctatgttgcctaagctggactcaaactcctgggctcaaacaatcctcccgcctcggcctcctgagtagctgggactaaaggtgcgcagcactatgcctggctgctttaagtcattattctataaagaattcttttcttttttttctttcttttttttttttcaagatggattcttgctctgttgaccaggtcagagtgcagtggcatgatctcagctcactgcaacctccgtctcccgggttcaagcaattctcctgcctcagcctcccgagtattggggactacaggcatgcaccaccatctctgcctaatttttgtatttttagtagagatggggtttcaccatgttgtccaggctgatctcgatctcctgaccttgtgatccacccgccttggcctcccatcgtgctgggatcataggcgtgagccagaattgttatctttccctgactaagaattaaatgtttttcttcttcaggttttttctttttttcttctttttttttttggtcttagtgccttctcatgtcactctatactgcccatctgaacaggccatctgactgatcttcactacttccttcgtttcagttaccataaattcttaatgaattctaaatatttacctttagaccaacttatttttgaatttcagatgtgaacattcaactgcttactgaaaatccccatcttaacgtcccacaggaacctccagaccaacatagtcaaaactgaaccccttagctccttatcaaatttccttattttaattcactatccattatttgtgtaagtcaaaacctaggtttttaacaagaagtctggcttaacatcacaaatcacattggcacatgttcattaaaataggatctggtctaggtaattgcaaagtttaaggctttcataatgtagcttatatatcatgatactgaactgtgttttatgaagcaataatacattataaataatgcagaaaattttataaagtgcctgatttttgggatatgctaagcaaactacaaaagtttggcacatcttgggatttaagccttttctatctattatttttcctgctctagcttattgaaacttgtgactagaaccttcccataaattttaatctgaagatatcaaatagccttatagatttaatttgtttgcatgtttcttaatgctttgaaacccgtgaagtaatcaagactattgctttttctagttggcgcatagcaccttttgtctcctctccatagcattgtcaagaaagacattttattctacgccagccatttatgtccagtcccactagtactcaaaatcttgaaattttaaattaaatttaatcaggctgttgtacagaattatttccactatcctaatgtcattatgcttcatatcattttaatttatatttaagtacaatttttgtttgtaatttttggtgctctgatctttaatacctggaaataattttagcaaaaatttatgtggtttacatttgggaaatgctttatctttctgttgaagattcacgatacatgctaacatacgtaaactctgaaaagtaaaagcttgtggaaaattatacttgttcaggttgtttaatccagcagttcccatggaatgtaaaaaatgaataacatttgtcatactatttactatttagtttgagagacctgcaactttaaaattgatactcagcttcgtgaataccttttctacttcagggactacataagagaggaaatctttcatttgtaaacattctactttgtagataatatgggagtctttttccttttcttaaatggcttccctcattgctcaccattgcgtcagatgacttccaaaattccttgctaatcagttttaggtgggcttgctttctgaaggtgagtttctctctttcccctgtccctatccttacattgccaagtctgtgtgtcctctttcttctgcctttttgctttcttcctttcctttctgacattctagattttgtcttgctgatattctttcagctttatagaatattattatttttgtgctaccacaatgattttacaagaaaacacgcgttaagggatagctgtttacctcgggatgcttcagttaacaaagtattttacgtagatttcctttagtctgaaatctctgtacttcagattagttccatcccctgacttttcagaaacacatcagttgtattaaaacgaagcatatctgtaacatgcttacaagtatgtgctaatgtaacagcactaagagaagaatagtattttggcagataaatttttaattaattgctgtaataaaatcagctaaaactctatcctcctcacagcatagaacatgtacttcagtttttaggtcagctcctacaagacaattaactggcaaggaagtcagtgctcttcattacatggatacaacaatatcagagaactgttagaataacttaaacttctctaatgagcttcagagctttaatgagaagattttctattcttatgtgattaatattgctctgtgatagaaagagaactttttttttttctgaaaggaaatgtagagcaaaagagttcaagataaatgattcagaggaagttaagaagtggttaatcccttatagcccaaaggggcttaattattgataactagtaactgccaccagcataggtaggctctccaaataatcattatagcctagaataaagtaacaaaaactatttttaaactaccagcattttaacaatttattttctgaattcagtctatcaagcaattagcttagatggctataaaactgtgtgagcttgtgaacctataagtttagagacacttatgattcacgattagtcttgaaaattttttatataaaaatagtcccagtaaatcactaagtcaaaatggacgaactggttcgatgtgtgtgtatgtgtatgtctaggtacacagctgtgtgtgtgtctaggtgtgtagctgtgtgtgtgtccacacacattttgaaaatcaacaggttagcagaaacaggatgacgaaggaagaatgagaaaaagagatagttataaagagtcactgataataagcaaaacattcttaggatatgtgggtgtatgtaatcagtggcctttttgtacaaatacatcttttttgacatattagcttccatcgacatcaaccgaagctgatttcagaatgtcaaggagaactagtgagaagtaaaagtgactgtaaaagtagtggaagtagaagcagtgaagatgtagagaagtttgtgaaagaagtaaaatagacgtagatggtcatcacatttaaataaaggttagtcatgcactttgaacagacactggttaaagatatttaggaaaatagaatggattttaaattttaattggggaagctgattgtaaagaagaaggagtaaaagcaaataccagaataaaaacactagagagggaaactatttcgttattagtttcagctatctccgaatgtgtgtttaggtttcaacttcagcaggagacatcaaatttattcagaagaagagctaaattgatgatggcatatcaattcaaaagctatgctttttgaagctttttgggaaagacattctcctgtgtgctgtcttttcactcccactgggccacataagaatgggcatggaacccttccttaccagtaggtaagggggcccacacaggaagtactgggcttatggccttatgtgggaatatcttttgctgtttcagaatcaacgtgtagttctttgttctgcttaagtgggtgtttcaatagtcctcagacatgtcctctccccacaattttttttcagataccatagaggagaggtcgaggtccttccactgaagcaccaagtggggtatataaagccacactgcccatgagggctgtggtgtgagtctgctagccgtgagagacagatgcattacaaagggatggatttagtgcactctttcctctcttactgtgagtagatgctgcacttcttgaacctggtgggagtgtgtgttgtctgttctcagagaccttgaatcatacacagtttttttttcacccccgggtgacatttacctccttttaacattggctacactcgtgtaatattctgtcctgcagcacagcctgaacagccagtgaacaatgaaacaaaacttcttcccaagcctcaatgaggattgtgccatagtttgaaggtaggagaatttcaagccattaatcgtctatgaatactgaataccagtgagaatggctccttcaggtctatcagcttattgctgcttctgtgttattcttaggccaaagaacaatggtcaatccagcaatcaactcattttggctgatttgtgcctttagtccaggcttctcagttgtaccacacagccgttcttctcttcaatttcagggtcattccttcaggaattctgtcaggaataagcagcaagtttcctactgctctgggtcctccttagacccttcttatttcatgttcagaggaatgaactccacatcctgtcctaaactccaccttccttctattattttttcatatttgacattgatactcattgaaactagaatctgaattattaccaccaaagatattacctttttttttgcaccggagagcttaatctttaaaatgttctgtgagagatgggaatggtgtactatctttctagtgtggcaaaagaagagagagtaacaaaataattgtgttcatattaagattatttgaatataaatattttgccaaatttgtatttaggtgtccatgttatttttaataattggattgatataagtagatcttaagaaatatcaaaaaaaaccgttttgtcaaattggagtcaaggaggcatggaaattctcttcatttgcagtactcctaaattcctgcttattctgtggttgaatttatatcaacaaagagaattggagattaaggcaattaatcagaaattttaattaaaaaaggaatggaaaaaataagtaaaatttcctatagaattctagggtttgacatctacacagatatattcattaggtagaataataagtcttttctttgctcttatgaaaatattgttctctaattgtcaatgagcataaagtaaaatgaagatagattatacaaatatccttattgattaaaagtttaaaattgccagtgcccaagaaatatgcctgaattcaaggatatcttattgcaaagaatatcttactgcagtttgttttttttccttactgtaaataaagggtcaaccaacagctaataactcacaactgcatcttttcttgttagaacatgtcgcccagcactttttgattagatgttctcagataatagctggggcagattgaccaaaggagagcaattttttttttaatcaggataacataatgattccatcacaaactttagaaagggctatggatatcaccaattttctttttttgtctattattagtgctgtatctttagtggaattgaagacatgaccttctgcaggtttcaatacaactttataaaaggctgtaacaatgcctttaaaatcaaatctattaactgttctacagagaagccagttttctagtaacttggccataacttaaagacagggaccctgaaaacaccaagcatctgcctgcaatctgaattatctccctgactagattcagtaatcttggctcttccctgcatttaagtcattttgtgtacatcaattttctcacctatgaaagaagagagttgtccaacatgttctgatgttctttcagttctgagttctttggataatttgattggagcccaagagatgtcctaaatctgactggcatgcagtaaatgtttgttgattgtgaaaaaatatttgacttccaaatgggttgtaaaatatctcgtgtggacatactgcagcaagttaacacttataattttgggagcagaataaatgtaattaagtcacaaatgcaaatgaattctctgcttatttaattcaaagagaaatagttgtctaaaaacaatctgacccatatactggttgaaaatagtaagcattatttttttatgaagggccaagtaagtggcttacatatattaaatgtaataaatgtattcaaaaagattttcacctagttgaactgcatttatacttcatgaatatcactagtgtaatcattactttaaaaacatatagctggagaaataagcatttagacaggtaagaaaacagactgaagttttaaattttatttttaacagtcattctaaaagaattatttatcattatatatttgtatgactgttaaaagttgaaacagaaaacatggaagtatgcttttaattgatcattgttatatatagagagtgaattaaaaaatttatgcatataaagttactaataattttttgagcacatatagtttcctacatacttacctatatctattctttaatccttgtaacagccccatgaactatctactattattatcatcattccatcttgcaggtgaggaagctgaggtctagagaagttcattaatttgactaaaggcacacagtcaataaatggtagagccaaggtttgaaccctggcattctggttttatgcttttaaccagctgcttattattggtatcttattgtgatgattttggaaaacactactaatttatttgaaatgtattaatagtctttatttatttacaactttaataaagaaaaagttaacctttccatatgttcaaacaagatgcataattttctcatttatcatccgcagtttctatttctctatgctttgtgaaatgaaaagctgtaatatcaaatttttattttgttttaatcatttgtatgatttgctggcctttgtgctattaaatagaatgtatgaatgcttttttagcatttagtatttaatatgtagggaacccatttctaatgaattgccatgagttgagaaatactctgtatgtactccctgaaattataagtagccaatacatgtctctatacattcctttaggaggccttattttgttctgttagaattattttaactcaaaatatattttgtttataaatgattggatcatctgttttactcaagcaattgacattgaatcacattaatattacttcattttttattcagaggttttaaacatctttcccttttactgtttttaaacacttaacactgacaatttttatatcttgtgatataaaatataataaattataatcaagtcaagaataagaatgaaatgaaaggaagaaagttgaaaaaatcagtcaaatggtgtacaatggtttcttctttaagtaagggaaataacagtttgtttacaatggaatctatctatttcaagaccctttaagaataatttgtgttgcaaagtgagaacatttgaagttccatttgaaaaatgcagtacttctgccctggcgaaaatagataagcttccgagattcttcttatgaaaaagataagcctgctgctactactgttgctgctgcaggtgccgcaaaaaggaaaaaaaaagtcttaaaaatatgttagtagacacatattagcaggagaacacatattagcagttccctagggctgtaaaaacaagcatctgccattgattttgtactcaattgaacttcacagagtcacagtcttacattattagatgaggaattatgtgggttttagtgaattcttaatgaaacctatattttaaaaattaaaatattttgatttgatcaatcttaagttgtatcacgttagtcttatccaaactcattcaaaatgtatatgggattgtttcatgaaattttgatttttttttgaaaattttgcatttcagagttgtaaatatagcttatctgaactctagaagtcatattgttcagtgttcctgaaagtaatgaaaaaagttatctagaatatgtacatatca

SEQ ID NO: 16 - DNA, 39754 bp, synthetic construct
  source 1..39754: mol_type = other DNA
ggaaacggcggcggcggcgcgggagcgggacagcggcgcggcgggcggcggcgaccggtgggtgccgaacggggcgcggggggcgcgggcggcagcggcggcactgagcgctccggcagcagcgcagttggaccgcaggctcgggcgctggcaagtcatgtagcaaaagcagcaaccacccctcaggaattagaaaaaaaaaaaaaaaggaaaagaaaaaagaaagaaagaaaaaagaaacaaccaccgccccgggggtgaagctccctcgctcccaactgtacttcttggtctccccccccaccccccgctcccccccccgctgcttcctcctcctcctcctcctctcgcagcagcagtcacagcagcagcagcagcagcccaaagggggggagagccgggcggcgggcgggcgcgtcccggagcggccgcggcgaggctggggagacagcgatcgaggcggctatagccacaattaattttccgagctacaggcgcacactagggctgtaaaaaaatttttggtttaacctttcctaccgctgggccaatcagagctggcggcgtgcgggagcgtctctctcgccgtcgaccaatcggaggggccgggcccacccacccccgccccgcccgccggctgccgccccctcgggcgctcgcctccccctctccgcgggcctcggaccccgaggccgcgcggagccgcgtagggatacgccggcgggagcacgcggtgccgcagcccttccctgcgcccccgccaccccctccaccccgccggctccgctccaccgaaattcccctcctagggccaatagctcacatttccggcttcctcctcattgtactgggtcattttcgtttaagcgtcaatatttgtccttagtgattaaagtttgtatccaattttatttttgaaagcgggggtggtggtggtagtgggaagagtgggagtgcagggagaggatgcgagagggcaggaaatggaggaggtagaggcgaggcactacttttccgagtcggccttgcaggccagtgtctggcatttctgagccagcaggaaaattgtgccctttgtaggtaatagtctagcgcagctttggataaggggggagataaggcccaccgctggcgtgtgtgcgggggtgtcctttacttcgggggaggagggtgtgggagagctttgggagcgatgagatgaggaggaggaagtgggcagaggagcgcggggccaaacaggggttacttactcgattcctgaggcctgggcggcggcgagctccgggctccggcaccgcggggaaaccgggactggggctccgggtgctgctgcggcggccggggccgctcagtagtgggcgcctctgatggccttccgaaatgagaggtgcccagagagaaaacagtaccaaacaatctcgaaataaatcaggtaacacatgctatatacgtgtgtctcttccttttccttctctcctgctccagagcgtcgctcacgcgcggggtgactcgatgtcgttttctcctcgccgctccagcctcagcttctaatccgaattggaagcgggtagctgcgagcgatcaccctggaaatattttcgttttgcttgttgacattggagggaaaataagggtgggcgtctatttctttccggttttttttttaagccttcctattttcactccttttttttcctttccgtttctcaaatgcttttattttgtgcgggcctcattcctgaaataacagtatactgaagccagctcttgccaaccctgagtgctgctggaaagcaccggggagccttgggaacgtggatctcacagtcccagcttcttgggctttggaactgggaaggttcggttggaggttgaagtgaatggggaaattgcgaatcgggggctttaaaaggatcccgtggccaggggcagggctaaggcccagcatttcccagggattggaatttagtggggagcacattgatcaaaggctgtttcatcccacatgggcacgaaacataccacattttacggaggatgtagccccagagaagagggaggtggagaagcataaatgaaaggcaccttttccttcctctagaaggtaaataattataaaatgtaagcatttcttcacttggataaactttcattaagatatctggcagatttggtatttaagaaaacgaaggaaggaaaattggtgcttatgcttgatcctaaagttaaattgtaacataaagaatgcggttgatgaatactcaggataatagggggttattccatcctccccacccccagtcttaagaaagagaaagagagaaaaggttgggggggggggaggaaactggagagaataatagataattacaaatagctagtgaattttaaatatgcatctattaggaatgccacaaaggagctttgaatattggactaacctcaaatgtccttccactgtatgtatgtatctttacctatgtctacatatcttctcactttaaaacaaactgtatttacatgtggaaaaattcaagggttccaacaatcagtgttgatagtgacatggtcccaaacatttatttccaagctctggatttctcgcagggccgggtgagctaatgggctttgcctaccggctggagagatttgtgggccttccccttcctactcacttttcctcctcccactcttctcccctcttcaggcccgttcttcctacttttcctcaactctccttcctccctcctccctccttctcccttagctccccccgccccttccccccaccctccattcctctgtctctccattcctccacctactcctcccaatttctcctcttacagccccccttctcaccaccacttataatggcacacagggctccctctgaggtggacggcccggtacgttttcgtagtcggtctagcgtgaaatgattcggtgtaaccggattcttgggaaaggacaggttacgtaaacatttactgatgtggagtaactatactatttgtactctgatatgcaggaaggaaagtgttgattaacacagaagaaaaaaaaaacgaaattagctgattgcatgctcataagtgcaagctgtcattgaactgcttttaaaatagacacagctgggtgtttgtgttgaaaaatttcttgtgtttttgctaatactgaaataagcatgattttgtataccaaacattttcagatcattaaaatttgtaatctgtgctggttttaaacacaaagaaaagaaacagaatcatttcgatgtaaaatttgctgatattatattaactcaatcaggtctgtacaaattgtgcattgtgcctcctaaaatctgtttgatcaatttaatacacaaagtgccaaacgtgggtattgtttgttaacattggacattttattcaaatccgagctcagcagcaaactggaggagctttccttcaatacaacaccagctctgactgatgaatttttaatgttttaaggtgtttcttaggtcaaatctgagcgtgcattgttttgtaatcctgtataaaattttagcaggtggaaataagtttttcttctgtttactttcaaatagcaaaaactggggtttgctaatgagcactgtaataatctgattttatatgctaggagctcatattgactgggaaaatgtttttcattcaaattaaggctatgttatatagtttaaggattgcatgaatgcaagtctctcaaaataggatattcttatgggcataaatagtatatagtttaatgtaatatttaaaaatctattcttcctaatgacaaaagttatttgtaatctggcaagaagtgagttttaattccttttaataatttcaattgcactttatgtcgagatggtgatatatatatatactcacacacatatatatgtgtgtgtgtgtatatatgtatatatatatagcttgttcaacaaaacatgcactgtttactcagcaccccgtgtttgtctcagcaatagcttttttaaagaactgctactatttgaaatggagggggaggggggtcctggacagagtattgtgcaagttgaaagtctctggatggggctttgtatatcctaccagccaatttgggtgcaaattggatttgaaggcctgcctctgtccacctcgggcctcttccctaagcactggaagcccaatcttcagccccactcagtcaccgtgattaccttcagaagacagagggaattcagttgtctcagacctgaaacaaattctctatcattctaatttttttctcatgatggatctaaccttgatctctcacacagacatctacaaatgtgtactattaatgaataaaagcagaagtcaaagtaaatgtaattttctggtatatatcacagtaaacttctctcttgctctgtctgcagtcccccagctctcccaaagaggtcaggccccgtaaagagtgagcagttccaatggaagaattggttaggaaatgaattagattattccttctggagttttaattaataaccaaggctacacagcatacttcatccgagtagggagcagaacatttcatttttccatttggcggtgttatttaagtattctttagggcctctttcctgttttcagtcagtgatccgcctcttttcttcttgctagctgctggttaaaggaaggtgggccccatttacctaaaatccactctgctctccattccctggacagggtgtccttggagtggcacccagcgccaagctctagctaaagttctcttcctgcagatgcacagagaatttctgaccttgtgacgtttgggagtgaggagatcccatacagaggcatccaggtatttccagagatcctgtggcaggtgaggtctgccctcgctggagccaactcgtctataatatcttcctaacagcaggtaatggtgctaacatttagtgattagaagttactctttagcgtgttgtatcctcgttctgtgggttttggttacaggttatacgattaggtttgcgagaacagtagtgaactaaacttctcagtaagaggtcttcacaccagggaagtgttacttttctgcagcagccctgcctcgtcactcattagggagctgcaggttgtcagtggctgctctcagcgcgcgcacgcgcaccctcaacgtccccgggggcgcgcacattcgcgcgtgtgtgtacacacacacacatacacacacacacacacacacagcactaaaactaaattaagaagccgatgattaagacaagcgtctactaggcctgtcatttcagcatccttccatcataacattgtggcaccggcaattttgcttgagaacctcgagtaaaaaaatctatgggagagcctcgtctatgagagtggaaatgtttagcttgtgtaagctagggagccactttaaaagggagaaagtccaaggcgaagaagaggagcaggaggaggaagaggacaaggatgaggagaaggccttgacagagacccctgggactctccaggcaagtctctgtgacgctgccaggtgttccaggctctctgaagccaagtacgtacgcagccaaaagaaaagtcccgtgatcttcagcaaaatgtgcatgggagaagctttgtcccgacattagccaagggagagccaggcgtggaaagatccatgacgaaataccaccagagggccaagggactttagtctctggaaaaacttctcagtcgaatactctgtacatctcttgtaaacattgtaaacataatgtccactacccttttacttgtttcgcgttctttgccagtgccctccagagggtgagtgatttgctgaaggacttgagtccctgatagtgtgagggcggacctgttccatttttagacctgatccagttgtgagaaatggtcccacacagcctggggaagtgcccctgagaggccgattcctatttttcctctagttacccaaggaaatggtgtagtcagtctttcaaaggagataggggaagctccacaaacaaccataacacagcacaacaaaacaaaacaaagagctgaaggggtgttggtgaactagaatgaaggaggctagagctcaggttcttagttttaacccccagctctatcccataaggaagaaagtgagacttggcaaaagccagcccacaaacaatattacctcggattttccatcagtggggcaccaaaacaggaaaaaagaaaataactatatatatctatatatatatatatatctatatatagatatatctatatatcaagctgaaagaaaaacttacagggctaaaaatgctgaactcttaagagctggaactcctaactatcccatcattgttcttacttagttggagcttcaatgtgatttctagtagaagcctcaatcttcttctggaagtgttacaggaccttttagagaaggggtgtgtgtgtgtgtatgtgggggtgggtggcgggtgaaccaagacttctcttttgaagcacacctccaaatggtcccccaaattccagttgtgtgagaaaagatggaaaggtctgactccttcgcaatttttagccggtcaccttcgaactagtcaccgctcaatatttaaatgtacacggaaaacattgaacagttcaaggtccgtatatgagattaataagtatcttataggaaatgcttgcacccactaaaggtctgaaggaatgagaggagagaaaacttcatgagtggggtaagcaagccaggaagcacgcagagaacccttcaccccaaagtctccctttgcgtggggaaagtagtatctactaatcagatctatctgcttctttcaagaaacctgcgtggacaaaccaatgggcaagtttttactttgtgctcatcttacacccacctggccctcactctccggagtcggttccagtaggtcttcctaccccaaccctcgctcctcccccgcgccccttctccaacacgaggccatggaaaatctgttggaggagcgtttctcaagaggtccagcttttctccctcccctgccttgcctcttgaggctgtaactgcgagggttcccttcgaggagcttcgcggagctggggctcccaggtctgcgcgccgctgctccctgcgcatacccgcgagcttgcccgacgctcctttgttgcagacgcttgccgaacgcaatcgagccagaggccttgattctaggcgtcatctctctagatgtccccccttaaagttgccttttcagcctaggtatctctttttttggcacataagaccggatacacaccaaatcaattttgggctaagaggtcatctgccacggtaaacgcggggatatagaaaacagttaaaggtttcaagtctgtattgctataaagcacatttcagagttaaaagactggggtgcatgcaggtgacgttaattcgaatattcaattacatgtaaggggtattgaacccagtgggggataacagtaatacccgtatttctctctcgctctttggaaggagtcgcctgcggggaggagaggagaagacagactaagctgcgcgtagagcggcatcaggagcaagttaccgttagcatgtgtaaacaaaacaactcgactcctctgtgtcagaatcaacaacatcaaaggtgtctgtggggggtcacttttatttttaaaacaatcctagattcttgttcggcaattttagtgtcttccttctgggtttgtcctaaggcacgtaggagagaaagtaatgttcgagaaaaaagctgggtcggcaagtttggctccggagcctatgaactatgaatcaccgcggtgaagaggttttcttttttctttcttttttttttttctcaccctcttcttttctcccggagcgtgaacttgttagataagatagatgaggctacaaggaaacagaaacgctgaaaaagcccagctcctttggagagcagcctgtggggctggcaggcggctgccgcgcgccctcccccagctgaggggcaccggggtagctccggagctgtcccaacttgggcctcctgggccccccgcggggccagacatcacgctcagatcgctgccttggccatcttaccttttggccatagaataaacaccccgaaagacgagagaaaggcctctatttgagctgagtgtctgctctcccgctcgaaacacaaaacagggcccgcaggttttcctagaggaagtcccaatcacaaaacagtcggacagtgggagacgctagagaattctggaattacacatgaaacattattttcctaattaattaaaaataattctcttttcctatttatatgcatacacacgctactgattacacgtccctatttacttgaaaaaatgcttggagtatctttagtacacaatgctcattctttgtccccttgaacatttacagctgataatgtggctggttgggatcaattagcactggattttgccccaggattgctccccagggcggacaagtggagccaactcatctccagcgacttttacttcgctcacgtgcatatccacgccagggctgcagaagcatttcaaaatccgcagccttggtttcacttgattttgttgttgttattgataaatattgggtcatacacaagccacttgtcagagcctttggctccacctcgcagctgcagctcctagcgggacgtttgagtcttgtcctaggaactggaaggatttactgggaaaagaattacacattctctgctccttcagttttggagatggtcccttaagatcatatcatatcgtctgtctgcttgctgtcacatttatttcttgattgttctgatatgtgaacaatgggaaggtgtgtgcatcttttccccctatctttttaaaacaattattgaaatggtttaaaagtgggggaggggagaaagtattgatcccagttctttccgtatatttggaaccaaaagaacataggcaaatagataattgttttaattgtcggggtggggggtgggttggtaggtaggacctttggggtttgtgtaattggagtaataaatacagtttatcttttagctccgccgtatatattctttcccttgatacatattgcacatgaaatgaacccatggtctacttgtcttagcatgtttgaagccccagacacccaacagtatcattacaggaaaaagacagagcccccaaagactgcatgcaggcaggaaggtgactgcctttggggccaatggggaatttatcttgacgttgaacttttctgggaagtgcacccactcgctcaggctcccttgctgcgcagagttcaggtcttccccatgctgaagagcagggaggaggtgtggaaaaaaacagctgatttttgacatttcaaagggactctttctatttatcagtgttaccgaagtgtacgttactggagaaacaacaaaacaagcatagagctgcaattttcaagaaaataggatccccaaatataaaaataaaaaagtagcagataagatgtataattcaatcaaattacacaaaattgcttgaaaatctagaacagtagcgatccattatgcctctgttttattagtgacaagtacaattactctaaaataaagaacactggtgaatatataattggctgtaatcatgttagtgtttgaataaaaatgacgggaacatttaacactttgacaacaaaatgttcagaaataacttatttattcaaacagtttttattttccaccaagtgaaggagaaacacagtttgcttaggaaaatcaatgacttttccctgcaataaataaatatccaaaagggggaaaaaacagccccacacctctatattattactgttattatttttaaccagcaagaagcttaatcctgtgtttgaagtcagcgctcatctagcggttgtttggtgccaaagtccgccacctagaggaagagctctgcagcccagaaccccttttcccgcggtgacctcgggaatcaaagttaatattcctttgcaaattgctctccaaactaactccccctccctttcctcctcacgtcccctttcgccctacaacatcgtcacaatattttcttagtaattgctctgcacaattgacagaagcgccgcgtctccggcagcgctgtgctctcccaggaaagcgtgagggcgcgttggggaagcaggtcagggaatatttggttctggagagacaatccactttcatccacaatggtctagagcagcgtgacttgggaacttataaagtgacggctgatggaggcatcgccacattccaatgcattttttaaccccgggctttcttagaaaaaacaactgggatccccaggtaccgcggggctattcccgacttttccccagcgcccccactccccaccccaaacgcgaagatctgagatctcggctttatttaatcatctactcagttattggagctcagtgggtccttttattctcctaggtgggttgcttgaagaactgagttacaaaggtattaaagaggaggccgtgtttgcaggggtccccctgtggtcagggaaaccgactcaggagaacccccatgtttttacactgagctgagcctccgagtttcaagaaacttgggcagtgtgcgagagaatttggggttgtactcgcaagcaccaggagtctttttgattttagtgttggtggatcctatcggctcctcacacagaaaagccaacctcactcttcaatctcgagctcatcctctcaagtaattattggggaagggagatgctaaacgagggagcatggggctctgggaagaaatttaacaacagcattcgagcgctcaaattctccttaacaccagtgctgcagattctgctgcgatccccgctctggccctccaggaatgtctccccgccccactcctccaaagttttgttttaattgctttagggcacttagaatattgttcaagttgaggatttcctagaaaggcgaggaagatagagttgatttagttttgaaaaaaaaaagttattaatcataagacttcagttgggtttcatttcttctaattttccttcctgtcccctccttccctccttccctctttccctccttccctctttccctccttccctccttccttccttccttccttctttccctgctttttcttcccttttctctttcctctctctccttctccctttccctctctctctcactatctctatgtgcgtctaactgtatctcatctacctccctaaatgtgagagaaatgaagcctcgtgaaggtgatggaggtgaccattttcattctgacttccagaacctccttacttgtcaattcttagtctcaatgctggagtttgaaagacatctcggaggtgattcttctccctcccagagacgacctcagaaccatggtctaagaagctatttttctgactgtgaaattattttttaaggcagctgaaataaatatatatttttaaaataagcctctgaaactgatgccccggtgcatgcttcctctatggattgaggcgctccctaaattcactccacccaaccccctaggagcttaagtcctcagaaatgttttgttttgttttatgcaaaactagcgcttccactctctactcccaggtctatcattcctctcccgcattatattaacggcgatgttagccttcccctacgtgtttaggctgctaatgccggccacttaacgcaacaaaaatgctcatctgtggagattttcccaaggctagctgcaatgttggctcctttttcttttcttttcagggcacgtccattcctccctgcccccttcttcttttcttgagatggggactggaaaaggggtccaacaactctaagcgagtgtgtgaagtttcccctctttctcttgcctgccaatccgtcaagcggggtggtgaatttttggttttctctagagagcaggactatggaagcttcagcgtagattggttgcctctcttagttaacttgccttcttcttcttcttttttttttcccccagtcatgactttcctttctcctcaccttagtcggtgaaagcagttggggattgccctcacagtccttctgcacccttcctagccccaggcttctgaagaaaactccactggctttcttttgtattgacagggaagctgggttggagatctcctctgtattggattgtgtgtgggtgttgtgtatgcttgtttagttttaataactttatgagggagagtctcctgagcttgttgacatttttcttctatggggagattttctggagcacggaatcaccttaatttagataatgaccgtctattaggcttcttaccctttcaagtaaatttagcctgggaaaatcatctgccctcatgattaatacatacctagtgtccaaggagaagaccatcaggggcaatgcaaactgaaactgacttaaagggtaataaatattacccagtgggttttgacttaaaaagatgaatatcgtcattgttcacagctggaacacgtttacttcttaaaagctggtgaagtgtagacacgagaactgttttcccgactctggctctagcactcttttcctatggatccttaggcaagttacttcatctttccagttttttcttaatctggaaaataagtgacctgcaatcactaatatataaaatatcattcattcatcaaattcccaacaacactaccactaaaatataactttaaaaaccataagaatctttggaaaaaatttttggggaggtggggctgggaagcaggaagaaaatctcattctaacagcagcagcgacatctagtgcccataatgctcaatagtagcagtgaaggtgtgggattgtttactgagtttttggaggggagagccatggaggcttttctaataaatcttgactgtctgtcgggcacacaatagtcttttgataataaaaaaaattctaagctttgattatggtaataagacatagagagacaacggtttttacctcaaatctgcacatagggcaattaaagtttgttatgagtattcttccaatgctttaaattccatgtcaaggaactagttctaaaaagtaattttattttgaggggggttgcaatttatttacctaattttccaaattatcactaaatatgtttctacacaaatacttaacaaaattgaaaaaaatttatttttgatattttcttttatctgaactaaccactttgattggagataattatctagtgaatagcagggaacatactaaaaataacaccatgttttaagtaatcacatatatgccctgtctatattaccttggctgaaaatattttataattcaaagaaatggaattcctatgtctatatggattctgtttcttcttcttacagcaaagattaaatgaactatctgggcacaggaaaaatcttagcaatggtagactgcagagccccaaacttttcagaataacaatttcggcatttgcttggcatttaagatgttagagagagagagagagtagacctcttctcattccctgtgactgtaagctggccatgtgcgtgggtgtagtttgtgcatgcacttgctgaagtgtgtgtttaatttcctaatagcaaagcagctgatgactactggtatatggattcatgttttgtaactttaagggaaaccaattttgtttattctatttttcaccaatatttcctagattttactataaaatttggtcactggcatgctatctagctagttctagaatagtaagataaatatacaagttgttaacacctaggggaatttgaggtagtcacaacacttctcttgaaccttagttttcttatgcacaaaatcagaaagttgaatctgctatcccttataattcaaaagatttttaagtcaataaatcatcaaccttcatttgagaagactctcttgtggtgtcttcattgttatttgcaactcttctctatcactcctgagttctctagttaggcatctcatcagaaatgagaattgatgagccagccctccatcttctctatcttcctgtcttgttctttccctttttctgcttttaaaaataacaaaaatctcagacactctagtcaccatttactaactcattggcatgcactcagctctcccttgctatccatccagaaaaatcataatgtgagataggaccaaactgaacacaaagctgaggcagctgtattcaagttttctaacatgttcccatatcctcatggtgatgattaattattattattattattattattattaggaaccagagccaaagtattctggctgcagatagcccacatttttctctctgcttctgtgtccctgtgaataaatttcttcaggaggatgcagtaaaatgtctccaaattcaatctgactaggaacatcattacacacttttagcaataagagtgttcacagaaggcaaatatcactccgtgatgcaaatatgctcagcatttaatatagacctcagggaagctgaagacacacacacatacccatgcacacacacacacatgcatgcgtgtgtgtgttgtatataaagtgtgtgtgtgtatatacatatacacacatatgtgtataaatatatgtatatggagtatcatatctacattgacctgggttgatagaaacaaacaagtttgttacagggaaaattctaaatatatcaaaggtttctttcttagatgcctttttgttgttccctagaaaacttcttagttcctactacttggacttttatttaaaattccttatgctgtacgaagttcataagacatgttttattctgcagatttttcacagacctcataaacacaggtattattctattttagaagtgaggaggctgacccagacagactaaattacttgcttaagatcactccactaggaagacacagaaaaggagtgtgaatcctggctttggacaggccgcaacttacacttggtcatttttagggttggagccttcaaactgggaaacaaggggattgactgaggctgttcattaaacttcaccaggattccaccgcatatatttgagaaatctcaacttttaaagaattttgggacccctagtctgtgtttgctactcctcattcatgtatgattttctcctcttaacactaacaattttatattctttctacaggaaggataatctttggaaaaaaggttgtactctaaacatttttgttaattatcttgggaactaagattttagagttggagggaatctaagacattttgtgattcagcagctctggagtaagatgattgtccgaatcaatggctcctgagaactgtgtgaaatgggagtccaaattattgtcagttctctggtgttgcttcctacccatctccctggatttttggctttatgcattaatacatttggttacttcattcttgctggccaccctgtggactagttttccaagcggctgcattttcagcagcacactgtggacttagccaagcactttaagctgagagctgttaagccttcctggatctattttctctcaagtagctctctgctgccctcaactggtgttcagccaccagagctagctagccttcagcatttacacgaatccaccgtaccctggcttcaagtcagatatgggagcatcattaaccacactgtttatggcttttatagcatggaaaaatagaacccaggcccattcacgtccacttatgcctcaggttctcactttctacaagtttgcagatgggccatgaaggagaatatctgattttcacttatgtgtgctggagtatttggaagggcttacatgaaaatgtaaactttctctttaattatcaaattgagatacacttcagctgatagaccaaatttttgaacatgttgcagttagtcttaagaaatacgaaacattttgtctttgatgtaaacaagatatagagaatcatatgaaatccatgaattacttgtaaactttctcatttgttcaataaatacatagcgtacacatactgtacgctagacattattattggtgctaagaatatagcattggataatggaggcaaaaacctcttctgcctttattaagctaacttcccagttaaggaaaatagacattaaacaagtaaataggtaaaatacattttaagggaatgtaacatttccatatctatttcctaccccaagtactgtagcgattgcatgtttttgttttgttttgttttgttttgtttgagacggagtctcactctgtcgcccaggctggagtgcagtagcgcgatctcggctcactgcaagctccgcctcccgggttcacgccattctcccgcttcagcctcccgagtagctgggactacaggcgcccgccaccacgcccggctgattttttgtatttttagtagagacgggatttcaccgtgttagccaggatggtctcgatttcctgacctcgtgatccaccccctcggcctcccaaagtgctgggattgcaggcgtgagccacagcgcccggccgcgattccatgtttttaagaacatttgtagtaaagttaaagaggcataactagagtttatacagggaccatgcatgctatataaatattggatgcgttatttattaatgaattcataaatttcttgtgaatatagagtagatatatgaaagtgtttgatgcagatgagtgtttgaacctttcagctgaagagacgttttgtcttttgtttctataccaatctctcctgttgaattgccttgtggttggaatatcactttgtcaccccctgtaatagctgccactaaagtccttgctattgctgctattgacgatggtctctggttattatctttacttttctctttgtttttattctgttctcttactatgtcttttgctttcttctcagataaactctgatagataccactgaccaaatagaataagttttccctttcttatgtggtggaactaccagtcataactagggaggtttaatcacctgattttcttttcactaccaggtctatttgaaatgggtatttgtcctaggtaaaatgcagctgatttggaagaaaccaaaatattaatcaaataataaccaagtggtagtggcaattacagatacacagcttctaagtaatatgtttgtatttaaataaataaaatagtccaaattctgaaaaaaaaaccctataatataaaactgagtttgagttgctgggtgagaagcttcatccttgccgtgaaaaatttttctgtgtggaactttccagttttgtttttatgatgcagacgtcttctgctaggcagacatgacaccatctcagtgaaccatgtaggtcagcacagggttaaaaatatgacgtacctatccttatatgaattagaaatgtttatatttattaacacatttcttgaaatatctttaatttttgtttacttctgattttatatatcacagacttgctgtaaaaaatttctagtatattataaagaagaaaataaacataaatcatctattaaagtaaaacctatattctaaataatttggatcatcttgtgggtatattttgtgttcagccttttgttcattattgttatactgtgagcatttccacataactatttttaaaacctatagttacaaatatctatattaacctatggataatgataaacaatttaaccactgtccattaaccattgcttcacatttaggtggtttctaatttttcattattaaaatgacttgagatatataactttgcacataagctttttctccaactttgatcatttataatagattttttaagggcaaggtttttttttttttttggtggggagggatcacatagtatgaatttgtaataaacatttaccatatactggtgaattgtatttctttaaaatttcattactgtggctgggcttggtggctcacgcctgtaatcccagcactttgggaggccgaggcaggtgtgtcatctggggtcaggagttcaagaccagcctggccaatgcagcaaaaccccaaccctactaaaaatacaaaaattagccaggtgtagggttgcatgcctgtaatcccagctactcaggaggctgaggcaggaggatcccttgaacccaggagacagagggtgcagtgacctgatatagtgccactgcactccagcctgggcaacagagcgagactctggcaacaacaacaacaacaacaacaacaacaacaacaacagcaacaaagattgtattagtgtacatttgcagtgatctataaaagtgactgttttggccgggcacggtggctcacacctgtaatcccagcactttgggaggccgaggcgggtggatcacgaggtcaggagatcgagaccatcctggctaacacagtgaaaacccgtctctactaaaaatagaaaaaattagcctggagtggcggaacgcgcctgtagtcccagctactcggtaggcttaggcaggagaatcacttgaacccgggaggtggaggttgcggtgagccaagattgcaccactgcactccagcctgggcaacagagtgagactctgtctcaaaaaaaaaaaaaaaaaagtatctgtcttaatgaaccctcaccaaattttaaattttcttttgccagtttaaaaggagaaatataatacctataactagctttaatgtacttttctttatgtaccagcaagattgaaagaagtttctacaaatatttattacattttaaatttcatcttttatgatctttctgttgtcttttgtgcattgcttatttgaatttttgtgctatttttattaattttaagggcttttctagataaaaatattaaccgtttgtcatctggctcaaatatcttttatagtttttattggtctttttagcctatttacaatatttagttatacacaaatgtaagttttatgcatattcagatattgaaaatgtaatgagcttgaaacttaaaacctgggtttgaatctatcactttgtgtctatgtgaacttgagtaaattattaaacctctgtaccttatttttatttgatataagatggggttaataatgctaattctctcataatgttttctgaggataaaatgcattaaatttataaggaatatagtttgaagtgtaccaggcaaatggtaagcccaaaattaagattgaacatttttattattgtttacagtattcaaatttacaaatgcaaaatgatttttattactttaatattagtattttacatatctaaaatgagttaaatactaagtaaagttttattaactaaatatcaaatatagctaagaattttgtatacctagttttttaaattatataaatgattaacttgagtattctgaggaaaaatactattatgtcttattcatgtacttaatactagttttattcattcaagagatatttgttgaacttttactacatgccagaaactgtagttgaacaagatagcgaacaaatggatgaaaacctttgcttttctgcaattgacggagaaaccataacaaaatacgttagtatcatgaaaaatataatagagaagaataaaggagataagaagtgcaaaaaaagtgcttgcaatttcaaatatgttagccaggggagatcactgagtgtattagttttctgttggtgcctaacaaattactacacatttaaggcttaaaacaacataaatttatttttttttccagtttctgtgggtcagatcaagagtctagacacaaactacctgggtcctttgctcaaagacttacaaaactgaaatgaaggtattgactgtgctctggttctcatctggaattcaggttgttgatggaatttagatccttgtggttgtaggactgtggcctctgttttcttgctgcctctcaactaaggtcactctcagctcctataggacagatccatccatcataatctgaataatcacccttttgattagctaaagaaaggagtgttggtgatcaattgcatcaaatgttgctgataggccaagtaaaatgagaagagaagattgactggatttagcaatacgaaggtcattggtaatcatgaggagaacagatttgttagaatggtggaggtaacagcctcttgggtgatgattaaaaatgaatgggaagaaaaatttgagatagtgtatagacagtttattgagcaggtttgtggaaagggaagcaagagaagaaacattagctagagaggaggtggagccaagagagggtttttttaatgggagaaaagtatgtttaattgctgatggaaatgatacagtagagaggtaaaagtaaaatctgatggcataggagagaaggggtagaattgctggaggaagtgagatgggatcaagtgaccaagtgagatggttggacttaggtgggagtactgataatcatctttcattccagggggacagcaaagtacatggattctgtaggttgtgggtattctcttctgattacttcagttttctcaatggactaggaagcgagctaaaactgagcatgataatggaagtgcttgaaatatgagtaggaaagattaggcatgaaaacgccatcaagaagagtgggagagtaaaagaccaggcaaatttaatatgattgccagacagtgttatggacatacatgaaatgcatgctcatgaatgtaaatgaaaccagtttacatgctgtgtgttttcctaagtcatgttagtttcagggccttcagaactagcaagacagaaagtaatatgtaaccagggttgtgattcagacaaatgactctgaacaagacaggtaagggaattggggatgcaaaaaggagtcactgcaatgattggtcatggaatttaaggtaaaaaggggggaaaaaggagagaagggggagagaaaagtgaaaagcataaattgaagacctaccaagcactatggtaaattattagaataaagtaatacaagcttttctttaaaactggctttctgatgactctaaggctgtgctgtatagagaggtcaagtgatcctgagctttagctcctcatgagctgcaggtaactcccatcacaatgacttattttgacaaactcaaagcactttcatagctcctatcccttggtacacttatgaagaaagcaagggcaggaactaatatctttgctttttatatagatgtaacatctttattttatatctagatatgcataaagagaggatccctggacacagtttttaatttaatttaatttaatttaattttattttattttattttattttattttatttttgagatggagtcttgctccgtcgccaggctggagtgcagtggcatgatctcagctcactgcaatctccacctcctgggttcaagtgattctcctgcctcagcctcctgagtagctgggactacaggcgcatgccaccacacccagctaatttttgtatttttagtagagatggggtttcaccatgttggccaggatggtcttgatctcttgtactgtgatccacccacctcagcctcccaaagtgctgggattacaggcgtgagccactgcgcccggctgacacagttctataatatacagaggtgaaattattgctaactcaaatcactgtttctctgtgtgactgctttcagcttccttagttctagtcctcagcaactcatgctgccctatggttgttccacattccccactgcaatcttcttctcaggattttagtcagcagccatgcactgtaattttctcttttcccaggcttccttcattgaaaacttcctgtgaagttctcagttctattgagtaacccattaaccactttggtaaacgaattccaacttgacctaagttcttaatcttgttatctgtcttagagtggtagttttcagtatgctcttccctcaacccacagtaagaaacacacatcatgacctaggccacaaatacatgtatctacatacacacatacacagctgaaacaaaaaattttccaaaatacccttattacttgtgatacactctgatagtttctattctattatattctatttaaaaatttactggttgagactcactaagctatttaccaactactaatggttgatggactgcagtttgacaataaaaggggcttttgatgcttataatgcaggtaggctataatcatggctcataacacatttaagtattttacaaaataactcctaattgagacctgataggatatgtgtattttagtagctgaaaagagctggaagttagcagttagtagaaagactatagaacttcaaaagacccaaatcctatacttcattctaatacttaataactttgacattggtttcttattctaccctctctcatgttctgtctatctaaaatggagttcaaaacacctgctacgtgtaaccctgaaatttattctcaagagcaagtgaactgtaagtaatggaggtttataaatagcaaatgtaagactctctaattttatttctggattgggtttatgttggaactctgaaagtcaggcaaaccctccaaagataagaatattccctgtcaccaactgctttgattgatcttaagttgccatatattattcaaatatgttgaattatttgaatattatattgtttcttatgtattcaaataaaatttatcatttatcagaaaaatattttaaagttaaaaaattatctacttgatatgatatggtgtataaagctttttatgcactttaacagaaaatgagtcatggttttttattaggttttgagataaatccacacaccaacctggaaggcatagttctaatacagctgctgtacattttcttcacagcttatttgtaatgtctttagtcattgttaggtctgttagtcagaagtttcacatctataatgagactcacttgtggtcatcatttttagtaattatttaaaaaaatgttgacatacaattttgtgtgttgaaattcagttctattcacagatttctccttctgttcttctgcaaatcaacatggataatctcaaaataacagtgcctctctacactctaaggatttaggttgtgtcatcatattaatattttgcccttttactatttccattaattaatttagccctttaacttttactgatcaatgtggaccaaattttaattgcaacattgctatgaaataataccaaattaaagctgatagtgtcatgctgtgagctagtgagagatattaacatttttgaacatgataagttgatttaaaaccagatggacttgtaaccaacttcagaatagtgttcaaaatctagttgtgaagaattttgaatattacattaatggttcaagtttggggtgtctgataaaatgctggtggcttggaaacatgtagctttacattatctctatcccattatactgttattagaaagttaaaacatgcaatgatgtaaagaaggaattcagtagtgtatctgatgaatatctattaattcccataacggatttaaatattaactttaatatagactgtgcaatatggttgattatttactcattaactaaacatttaatcattatctactctgtacaagatactacacgaggtgatgaagacagagtgttgaataaaatttagttcctactccaatgggactcacagtctggtggaggagatgaacacacagtcatatgtatagcataaaatcagtgccacactggaggtatgtactgggtgggttctatggaagtacagaagaaagccacttaagtcttcagattgtttcatggaagaaatgtcattaaatctctgtcttcaaaaattatcttaaaaatttatcgatattgtacatgaaaacataataataaataatttagaagagcttacgataaaaataacagaactgtttctcttcaacagctgtttggattcccggctgtgaactcttttaaatacctttttaaaattttctttcttttttttttttttttttgagacagggtctcactctatcatccagcttggagtgcagtggtgcgatcacagctcactgcagcctcgacctcatggctcaactgatcctcctacctcagcctcctgattagctggcaccacaggtgcagcccaccatacctggctgattaaaaaaaaattgttgagatagggttttgccatgttgcccaggctagagttttaaatacttcgtttgcatttctttttgtgattccttcatgatactaattatattatacttattatgggtggtttagagttatttatcatttcattacttccacaggttacttttgtgataatagtaataccctaacacattatgagatattatcatccccactctttcatccaccttttacctcccttagttcccaccatttattagctaacctagttttattttattaagactattataatttgtactttgaaattaaacagcaaccaaatctctcaccctttctccatgaattaagtctggaaatttacatttttatgactatgctgagtcatgcattgtgctatgacagaatacattctcttttctcactatcaattgcttaaaaccatgccagagatttaatttttttccatttgtatcgtagccttttacacaactttcttcctttattttctttaggttagccatcaacttttctttctttctgtctctgtcttttttggcttcactatattagcacattattgttttaaagaggtaacatgtctttattgcgtttttcacacattctcccagtgattctcaaatggggaggtcttttctcacatgccttcctcctgacaccatcaatccttcctgccatacaggaatatcctgcattgctttcctgggtgtgacccacacttttttcctgtctctcctcttctttcttctctttattttcttcttttttttcttgtttcgtttcttatttttctttattttctttaatttctttatttattttctttaaaaggtatgtggtaaatacataccatattaccaaatgcttttattctggtgtgtatgtgtgtgtgtatgtgtgtgtatctgtatgcgtgtgattgaactgtgtatatcatttttaagtcaaaaatattttctcaaaaactgtaaggttttggtctattttcttagcattcagtgttggtgataagtttaaactaatcctttctctctctctcttttttttaatagaacctgggtctcactctgtcccctaggctggaatgcagtggtgtgatcatggcccactgcagccttgaactcctggacccaagcaatcctcccatctcagcctcccaagtagctggaactacaggtgtaagccacacctggataactcatttatttttagataacctgtttttaatattggaagcttttatgattatctctgggtaggggtctttacattccacttggcatttttcactttaaattctcatgtctttcaacttggaagaatttgattctatcagttctctgttgatttcttctactccattttctgtattcacgttgtttttgggtttctattactcagatatggggcgtccataccacttctataggtcttttatctgttccctcatgctttctgactctgcatttctgctttacactgtaacagagttctctattttattttctagatctttactgttaattccgtttagtagtttgttctgcattttccagttgttagaaatctgttattattcctcattttttgaaaacttctttcctacattcttcttagttacgtattaattctatttttgtgcatttatgttcttattttagtgctgtctcaggagagataagatgtatcgctaagacctagtatgaatttgagtcagaactataagcagcaacaggttgttttagggttaaagtaacaaactagaaacagtagggccacaatttgcttttttttttttttttttttttgatggagtctcaaatctcgctctgtcactcaggctggagtacagtggtgctatctcagctcattgcaacctctgcctcctgggttcaagcgattctcctgcttagccccccagggtagctaggagtacaggcacacgctaccatgaccagctaatttttaatttttggatttttggatttttagtagagatggggtttcaccatgttgtccaggctggtcttgaactcatgtcctcaagtgatctaccctccttggtctcccaaactgttgggattacaggcgtgagccactgtgtctagcctgctgagataatttctaggtgtcatatcatggagggtcttgtagatacactctactaaggacactggacttttatcttagaggcactgaatgttctaattgtgaaaatagtatgatcttgtaaagaaggtatatttaaagactagaggccaggaggacagttagaagcctgttgaaagaatttatgttagtgataaagatctgatccaaagcagaaagatcagagagaagatgaggcaaaaataatagagatactacagaataaatttccaaaattgagtaaaaatatgaagacaggaaatgctaagaacgccaaataggataaaggcagagaaaaccacatgtagtcacatcacaataaaacttccaaaaaccaaagatgaaaaaaagcaaccttaaaaacagccaacagataatcaaacaatatatattaccttcaaaggagcaattaaaggcaaaataagagtgttacctttcttctcaaaattagtaatgaaatccagaaaataatagaatgataatcctaaaaaagctaaaataaaatacgggacagcttataattaaagcctagtctctctctacttttgcctaatgtactatgtccctgttcattaaggcaatatttcgtcttaatgaaatgtccctgttcattaagaaaatataagaaaatactttattctcttataattttgtggagctgatgttcataaaatgcatcatttaaatagttaattttgtaagatgatgggcttggcagagtaactgagatttaaatccttgctgtttaatctgttctaattggttaagggtaatgcccaatttagaagcaaaaagtaaaattgcagatttgtttttaactcccttcagttctgaagtttaatagtttcaaaaacatctcctttattatacttaccctaagaaagttgactcagattaattgtggcaatatttgataaacataataatgccaatattagatgaaatgaaaataattgtgaatagaaggctgtgtgtaataaggggattataaagacgtaggttcagagcagacctttatttatttacttaataggtgagtactggttctgttgcctagtagagtgcagtggctattcacaggcatgaccatggcgcactacagtcgcactcccaaatactgggactacagatgggcaccaccacacccagctgtagagtggatcttttttttctttttttttcttttttttttttttccccgagacggattctcactctgtcgcccacgctggagtgcagtggcgcgatctccactcactgccagctccgtcttccgggttcacgccattctcctgcctcagccttgtgagtagctgggactacaggcgcccgccaccacgcccagctaacattttgcatttttagtagagatggggtttcaccgtgttagccaggatggtctcgatcttctgacctcgtgatcctcctgccttggcctcccaaagtactgggattacaggtgtgagctaccgcgcccggccaagtggatctttgtaagaactgtgggatagcaaatatagaattagggctgtgcaattttatgtccagatagactattatgaaaataaaattgacctccatgagctctggagttagatttcctgtgtaaaaactccagctcttcaatttaccagctgtgtgaccttggacaagtttcttaatgaatctgcatttcactttctgcatctgtaaaaccttttcaagattgtggcataaatttgtaagcatacttcaaatgttaagcctttattataataatgaaaatttggcaaaacaaagctgatgcattcatagtaattgtgtaatgtatttattaatattttattggattctggtatttggcttaggcaaaggaagtatctgaaaatatttacgttttatgtaatgttaaaagaatgaaaagaaaaacaaaaaaaattatcaatatatgctaagaattttgttgacagtccacatatataattttcttaaccctaaaatgaatttataatgtaggtgttattttttgttattttacagacgggaaaactggcttagagatgttaaatatcctggattattgtcagtttaacaaaataatttcggctatcaaagatcaagtgggataaatttcagaggaagaaactgagctccttgcgtaagtcctagaaaaaacataaaaggtggttaactgcctccatataaagctgattttttaaaaagctctcattttaggtttaggggtacatatatggagttgttatacagataaattgtgtgtcatgggggtttggtgtacagattatttcatcaccctggtaataagcataggacccgataggtagttttctgaccttcttcctcctctcacctcaccctcaagtaggcactggtgtctgctgttctcttctttgtgtccatgtatactcaatgtttagctaattctagcctgtaaatttaatcacaaatctcctgttaacaatgctgtgttgcttccttctttggctatttcaactatctgctgatttcttttctttttactactatttcatttgcagtggagccaaccaatcctaacaaaattgaatagtagataaattataccatttcatctgaagaaatacctgtctcatttttactttttctctcactctatcctctactaaaaactgttttcagataaacttctgaaaccaatcatttataactggatataaattcaaattatttttacttctcagaatattatctcaagctaacttgaattgggtaataaaagacattttactctattaaagaaaagctaggcatttcctgaaatatcaattagtttggaagcaattcacctatatgtcatgtgaaccctaaaataaaataaaaaaatctagactggttatgtatattttcctgttgaaaaagatctagatgattttttaaaatgctacatgttactttctaataaatattttttaaaaagttggtcaccagactgcccctaagcaataagagatttccaaatttttatataaagtggaattatgaaacctaattatgtacaactgctgttctaccatgtttgggctacattggtaggaatacctgttagctgtgcattgtcaaagattgcttatacttggagatatgaatgtttggccactcctagttggagccaggcctatattgatcaaggataatgaaaggaagtactgatgatggtatttctttgacagtttcagttaacaggaagtattagtctgaattagggtgtaacaaagggaacaatgattcagtaagttaaagaagatagaactggttttgtcctttacataacaatccaaattgaatggttcaattctgctccatgaagttattcaggaattaaggttctttccattgattgcagtattatcctctaggactgtcatcttctgcatggcagagacagggtcagctgccattccagctggtgataaggggaaagataacatggaagcatacacacggtcctaagacttaggtccataagggacacctatcacattaaattccatggggaaaagaagtccactatgaaaaggaggctggaatattccatttattcctgcatgcaggaataaaaggagagtatcttttggtagacaactaatactctccactttatggtttgtgctgtttgttgaccctattgaagtttcctgatgatgtgcagtcttttctttgtgaaaaggataaagattgatacggtttctgaatggacccttaaattggacgaataataccacccttatttttctgtagactgtgttttacacttgcctatactgtgaaaaataataatcttttattggataagagtctctccattcttattttcttcaacttatttggtctcgggaaaataggaccaatattaaagctgtcttgtgatttacctatgccttgtgaatctgtttctagtcagtttcacttagaaggaaaagggccagattctacctctaattctgcaaggaaaaatacattcttagtagatcagaatacattttaatctacccttttcctgaattagattaaaaaagctttgcttactaaatagcttacaaagtgtcttcttttgaaataacctgtacagataactttgacagggaatttactagggaaattaaacaaagctgcctttattgttaatgaataattcttcaactacaatttaatattagatatactatatttaaaatgaagagagtaatgttcattcttgtaggatagtgattattttctaattttctattaggaccaaacttatgttttgatgtaatgttgggttttgtatgtcacaagtctccagcagatccagaaaggcccttttcacctgttactatgctcacttactagctgatgtctaagtttctctgctagaaattatttatcttggcttaaggctgtaccttgctaaaacccaattatctgtgaaaaaattaaccttttaaactttataatcattttatattttgttctggctgaatttgttgttgagatttttgaaaatgagctagaagaatccttgaggtggctgtgagagggtccactgactatggaaatgaacacaaaaagatttttgtttaaaagatgaatttaatttgtgaattttctctccattgtaggtcactttgagttctggctgtcttacagaggaaaaaatagagctgatcatgaccattataaaaacgtattatgtgcaaaagatgtacatcattccctgaatattttggtttttacttcaaattagaaaattactgttgttgatttaaataaaggacagcatcaacaaaatcatcatgaattggaaatagtgaaatttaatttttataaatctgtgctaaacattttaatggaaatatatttttaatatatattttaatgaaaaaattttaaaatatatattttaatatatattttaaaaatatattttaatgaaaaatgtagtggtgacaaattcaaacaagtaaaataggatttaagttatatgtgttaataaggatattgatctactgttgatattctgaagctattagcaatcaaatgttaccattaattttcagcccttgagctactacttagttttctactcaccagttctaccttttaaaaaattgcaaacaacatggctgtatgatgagggaaaagctttacccctacttggacaaaaaataggccctgcttgtaagattagttgaatgttcaggtccttgtaagttgtaaagaatgaagtttaaaaatttgtgttaggggccgggagtggtggctcactcctgtaatcccagcactttgggaggccgaggtgggaggatcacgaggtcaagagatcaagaccttcctggtcaacatgctgaaaccccgtctctactaaaaatacaaaaattatctgggcgtggtggcatgcacctgtagtcccagctactcggtaggctgaggcaggagaattgcttcaacccgggaggcagaagttgcagtgagctgagatcacgccactgcactccagcctgggtgacagagtgagactctatcccccccaaaaaaagaaaatgtattaggtacatttttttatactttaaatttgatttaagacattatcttgactaccaataactctactgctgtgaatccttgaaatttaatgtttagatttagatctaatagataactacattgtcaactctgtaaatgcttggccattttattaatgttgctattttctagtgcttgcatatagtagacagtaaataagtattgagtggatgtccaataactctttgtcattttaatttgtaagtaattattaattctttttctttaggatacaggctaaacaagagtcaaaggtaagccaaatgagctggagaacagaggagagattagacaagggcaagtgtgtataaggtggtaaaagagtaccctgagataaatgtaaatggtgtgcaatagaggtttaaaggatagagcgcttgtacacagtttggcaattcaggaatgcctccagtaggatatggcatttgaggttgaacctcgcagggtttgtgtggctgagtggtgaattgtgaggatagtcatggcagagagcacattctataaagattagagcaaattcagatgttttccatctctgaccttgtatggttattttacagcacttgtgtcacacactgttttaattattttgtactgtttatacctctcatgccatccactaaattataaacttcttgagggctatgtgttagtaagtcttgtattttctgctaagtctatgagtgctctgtatacattaatatttttaaaaaattactttatatctttcaagttatcaaagattttatagttttcctaattacgttgaatctagtgattaacaaatgaaatagctccttgaataggctgcacaatcaagcatttctaaaacagcagtttagagctaaggcgctggagttagaattgccatctttaaatactggtttagctgttgactaacagatcttgacaatttgttaaccacagtggactcagttttcttatctataaaaagggataatattcatacatacctaatagagctctgtggtagaatatagattgctataaatactttgctactcttccttttgaaagatggagtctaatattcatccctttgagtctgggctggcatttgtgacactcttaaccagtaaacaataatggaagtgatattgtgagacctttgaggctagatcatatgaagctctgaagcttttgctgatctctttttgaatgctgctcttaaaaatctttctgttggagcctttagcctccacataaggagtttgattactccgaacctttagccatcatataagaagtttgactactctgagactcatgagacatgggatctcatgctggagatccacgcgtggattctctggttaacattactagctgagctaaactttttggccattccaggcaaggcatgagacacgtgtgttaagcctcctggtatcctttataacagcccatccaccaggtgaatagcatctggtgatctcagctgacttcatatgtagaactagtatcacctattataacccaactcaatttcttgacccactttcattaaccataaaataatggttgttgttttaagacaccaagttttgaagtagtttattacatggtaaaaaataactggaacaggctattatgaagattaaatcatttgctgtatataaaacagcacagcgctgggcacatattatgtattctataaaagttattgctattatgatattattacaccataaccacaattataaagtctgcatttgtgtgtgtctgtgtatgtcatgtttgtgtatgttatttgcaagtattgattaaatggacttgaaatgaaagccaataatatgctgcactttcatataagagaagctttaagtcatttttttaaagatagggtcttgctatgttgcctaagctggactcaaactcctgggctcaaacaatcctcccgcctcggcctcctgagtagctgggactaaaggtgcgcagcactatgcctggctgctttaagtcattattctataaagaattcttttcttttttttctttcttttttttttttcaagatggattcttgctctgttgaccaggtcagagtgcagtggcatgatctcagctcactgcaacctccgtctcccgggttcaagcaattctcctgcctcagcctcccgagtattggggactacaggcatgcaccaccatctctgcctaatttttgtatttttagtagagatggggtttcaccatgttgtccaggctgatctcgatctcctgaccttgtgatccacccgccttggcctcccatcgtgctgggatcataggcgtgagccagaattgttatctttccctgactaagaattaaatgtttttcttcttcaggttttttctttttttcttctttttttttttggtcttagtgccttctcatgtcactctatactgcccatctgaacaggccatctgactgatcttcactacttccttcgtttcagttaccataaattcttaatgaattctaaatatttacctttagaccaacttatttttgaatttcagatgtgaacattcaactgcttactgaaaatccccatcttaacgtcccacaggaacctccagaccaacatagtcaaaactgaaccccttagctccttatcaaatttccttattttaattcactatccattatttgtgtaagtcaaaacctaggtttttaacaagaagtctggcttaacatcacaaatcacattggcacatgttcattaaaataggatctggtctaggtaattgcaaagtttaaggctttcataatgtagcttatatatcatgatactgaactgtgttttatgaagcaataatacattataaataatgcagaaaattttataaagtgcctgatttttgggatatgctaagcaaactacaaaagtttggcacatcttgggatttaagccttttctatctattatttttcctgctctagcttattgaaacttgtgactagaaccttcccataaattttaatctgaagatatcaaatagccttatagatttaatttgtttgcatgtttcttaatgctttgaaacccgtgaagtaatcaagactattgctttttctagttggcgcatagcaccttttgtctcctctccatagcattgtcaagaaagacattttattctacgccagccatttatgtccagtcccactagtactcaaaatcttgaaattttaaattaaatttaatcaggctgttgtacagaattatttccactatcctaatgtcattatgcttcatatcattttaatttatatttaagtacaatttttgtttgtaatttttggtgctctgatctttaatacctggaaataattttagcaaaaatttatgtggtttacatttgggaaatgctttatctttctgttgaagattcacgatacatgctaacatacgtaaactctgaaaagtaaaagcttgtggaaaattatacttgttcaggttgtttaatccagcagttcccatggaatgtaaaaaatgaataacatttgtcatactatttactatttagtttgagagacctgcaactttaaaattgatactcagcttcgtgaataccttttctacttcagggactacataagagaggaaatctttcatttgtaaacattctactttgtagataatatgggagtctttttccttttcttaaatggcttccctcattgctcaccattgcgtcagatgacttccaaaattccttgctaatcagttttaggtgggcttgctttctgaaggtgagtttctctctttcccctgtccctatccttacattgccaagtctgtgtgtcctctttcttctgcctttttgctttcttcctttcctttctgacattctagattttgtcttgctgatattctttcagctttatagaatattattatttttgtgctaccacaatgattttacaagaaaacacgcgttaagggatagctgtttacctcgggatgcttcagttaacaaagtattttacgtagatttcctttagtctgaaatctctgtacttcagattagttccatcccctgacttttcagaaacacatcagttgtattaaaacgaagcatatctgtaacatgcttacaagtatgtgctaatgtaacagcactaagagaagaatagtattttggcagataaatttttaattaattgctgtaataaaatcagctaaaactctatcctcctcacagcatagaacatgtacttcagtttttaggtcagctcctacaagacaattaactggcaaggaagtcagtgctcttcattacatggatacaacaatatcagagaactgttagaataacttaaacttctctaatgagcttcagagctttaatgagaagattttctattcttatgtgattaatattgctctgtgatagaaagagaactttttttttttctgaaaggaaatgtagagcaaaagagttcaagataaatgattcagaggaagttaagaagtggttaatcccttatagcccaaaggggcttaattattgataactagtaactgccaccagcataggtaggctctccaaataatcattatagcctagaataaagtaacaaaaactatttttaaactaccagcattttaacaatttattttctgaattcagtctatcaagcaattagcttagatggctataaaactgtgtgagcttgtgaacctataagtttagagacacttatgattcacgattagtcttgaaaattttttatataaaaatagtcccagtaaatcactaagtcaaaatggacgaactggttcgatgtgtgtgtatgtgtatgtctaggtacacagctgtgtgtgtgtctaggtgtgtagctgtgtgtgtgtccacacacattttgaaaatcaacaggttagcagaaacaggatgacgaaggaagaatgagaaaaagagatagttataaagagtcactgataataagcaaaacattcttaggatatgtgggtgtatgtaatcagtggcctttttgtacaaatacatcttttttgacatattagcttccatcgacatcaaccgaagctgatttcagaatgtcaaggagaactagtgagaagtaaaagtgactgtaaaagtagtggaagtagaagcagtgaagatgtagagaagtttgtgaaagaagtaaaatagacgtagatggtcatcacatttaaataaaggttagtcatgcactttgaacagacactggttaaagatatttaggaaaatagaatggattttaaattttaattggggaagctgattgtaaagaagaaggagtaaaagcaaataccagaataaaaacactagagagggaaactatttcgttattagtttcagctatctccgaatgtgtgtttaggtttcaacttcagcaggagacatcaaatttattcagaagaagagctaaattgatgatggcatatcaattcaaaagctatgctttttgaagctttttgggaaagacattctcctgtgtgctgtcttttcactcccactgggccacataagaatgggcatggaacccttccttaccagtaggtaagggggcccacacaggaagtactgggcttatggccttatgtgggaatatcttttgctgtttcagaatcaacgtgtagttctttgttctgcttaagtgggtgtttcaatagtcctcagacatgtcctctccccacaattttttttcagataccatagaggagaggtcgaggtccttccactgaagcaccaagtggggtatataaagccacactgcccatgagggctgtggtgtgagtctgctagccgtgagagacagatgcattacaaagggatggatttagtgcactctttcctctcttactgtgagtagatgctgcacttcttgaacctggtgggagtgtgtgttgtctgttctcagagaccttgaatcatacacagtttttttttcacccccgggtgacatttacctccttttaacattggctacactcgtgtaatattctgtcctgcagcacagcctgaacagccagtgaacaatgaaacaaaacttcttcccaagcctcaatgaggattgtgccatagtttgaaggtaggagaatttcaagccattaatcgtctatgaatactgaataccagtgagaatggctccttcaggtctatcagcttattgctgcttctgtgttattcttaggccaaagaacaatggtcaatccagcaatcaactcattttggctgatttgtgcctttagtccaggcttctcagttgtaccacacagccgttcttctcttcaatttcagggtcattccttcaggaattctgtcaggaataagcagcaagtttcctactgctctgggtcctccttagacccttcttatttcatgttcagaggaatgaactccacatcctgtcctaaactccaccttccttctattattttttcatatttgacattgatactcattgaaactagaatctgaattattaccaccaaagatattacctttttttttgcaccggagagcttaatctttaaaatgttctgtgagagatgggaatggtgtactatctttctagtgtggcaaaagaagagagagtaacaaaataattgtgttcatattaagattatttgaatataaatattttgccaaatttgtatttaggtgtccatgttatttttaataattggattgatataagtagatcttaagaaatatcaaaaaaaaccgttttgtcaaattggagtcaaggaggcatggaaattctcttcatttgcagtactcctaaattcctgcttattctgtggttgaatttatatcaacaaagagaattggagattaaggcaattaatcagaaattttaattaaaaaaggaatggaaaaaataagtaaaatttcctatagaattctagggtttgacatctacacagatatattcattaggtagaataataagtcttttctttgctcttatgaaaatattgttctctaattgtcaatgagcataaagtaaaatgaagatagattatacaaatatccttattgattaaaagtttaaaattgccagtgcccaagaaatatgcctgaattcaaggatatcttattgcaaagaatatcttactgcagtttgttttttttccttactgtaaataaagggtcaaccaacagctaataactcacaactgcatcttttcttgttagaacatgtcgcccagcactttttgattagatgttctcagataatagctggggcagattgaccaaaggagagcaattttttttttaatcaggataacataatgattccatcacaaactttagaaagggctatggatatcaccaattttctttttttgtctattattagtgctgtatctttagtggaattgaagacatgaccttctgcaggtttcaatacaactttataaaaggctgtaacaatgcctttaaaatcaaatctattaactgttctacagagaagccagttttctagtaacttggccataacttaaagacagggaccctgaaaacaccaagcatctgcctgcaatctgaattatctccctgactagattcagtaatcttggctcttccctgcatttaagtcattttgtgtacatcaattttctcacctatgaaagaagagagttgtccaacatgttctgatgttctttcagttctgagttctttggataatttgattggagcccaagagatgtcctaaatctgactggcatgcagtaaatgtttgttgatt

SEQ ID NO: 17 - DNA, 34275 bp, synthetic construct
  source 1..34275: mol_type = other DNA
cccagtcgtcggggggaaacggcggcggcggcgcgggagcgggacagcggcgcggcgggcggcggcgaccggtgggtgccgaacggggcgcggggggcgcgggcggcagcggcggcactgagcgctccggcagcagcgcagttggaccgcaggctcgggcgctggcaagtcatgtagcaaaagcagcaaccacccctcaggaattagaaaaaaaaaaaaaaaggaaaagaaaaaagaaagaaagaaaaaagaaacaaccaccgccccgggggtgaagctccctcgctcccaactgtacttcttggtctccccccccaccccccgctcccccccccgctgcttcctcctcctcctcctcctctcgcagcagcagtcacagcagcagcagcagcagcccaaagggggggagagccgggcggcgggcgggcgcgtcccggagcggccgcggcgaggctggggagacagcgatcgaggcggctatagccacaattaattttccgagctacaggcgcacactagggctgtaaaaaaatttttggtttaacctttcctaccgctgggccaatcagagctggcggcgtgcgggagcgtctctctcgccgtcgaccaatcggaggggccgggcccacccacccccgccccgcccgccggctgccgccccctcgggcgctcgcctccccctctccgcgggcctcggaccccgaggccgcgcggagccgcgtagggatacgccggcgggagcacgcggtgccgcagcccttccctgcgcccccgccaccccctccaccccgccggctccgctccaccgaaattcccctcctagggccaatagctcacatttccggcttcctcctcattgtactgggtcattttcgtttaagcgtcaatatttgtccttagtgattaaagtttgtatccaattttatttttgaaagcgggggtggtggtggtagtgggaagagtgggagtgcagggagaggatgcgagagggcaggaaatggaggaggtagaggcgaggcactacttttccgagtcggccttgcaggccagtgtctggcatttctgagccagcaggaaaattgtgccctttgtaggtaatagtctagcgcagctttggataaggggggagataaggcccaccgctggcgtgtgtgcgggggtgtcctttacttcgggggaggagggtgtgggagagctttgggagcgatgagatgaggaggaggaagtgggcagaggagcgcggggccaaacaggggttacttactcgattcctgaggcctgggcggcggcgagctccgggctccggcaccgcggggaaaccgggactggggctccgggtgctgctgcggcggccggggccgctcagtagtgggcgcctctgatggccttccgaaatgagaggtgcccagagagaaaacagtaccaaacaatctcgaaataaatcaggtaacacatgctatatacgtgtgtctcttccttttccttctctcctgctccagagcgtcgctcacgcgcggggtgactcgatgtcgttttctcctcgccgctccagcctcagcttctaatccgaattggaagcgggtagctgcgagcgatcaccctggaaatattttcgttttgcttgttgacattggagggaaaataagggtgggcgtctatttctttccggttttttttttaagccttcctattttcactccttttttttcctttccgtttctcaaatgcttttattttgtgcgggcctcattcctgaaataacagtatactgaagccagctcttgccaaccctgagtgctgctggaaagcaccggggagccttgggaacgtggatctcacagtcccagcttcttgggctttggaactgggaaggttcggttggaggttgaagtgaatggggaaattgcgaatcgggggctttaaaaggatcccgtggccaggggcagggctaaggcccagcatttcccagggattggaatttagtggggagcacattgatcaaaggctgtttcatcccacatgggcacgaaacataccacattttacggaggatgtagccccagagaagagggaggtggagaagcataaatgaaaggcaccttttccttcctctagaaggtaaataattataaaatgtaagcatttcttcacttggataaactttcattaagatatctggcagatttggtatttaagaaaacgaaggaaggaaaattggtgcttatgcttgatcctaaagttaaattgtaacataaagaatgcggttgatgaatactcaggataatagggggttattccatcctccccacccccagtcttaagaaagagaaagagagaaaaggttgggggggggggaggaaactggagagaataatagataattacaaatagctagtgaattttaaatatgcatctattaggaatgccacaaaggagctttgaatattggactaacctcaaatgtccttccactgtatgtatgtatctttacctatgtctacatatcttctcactttaaaacaaactgtatttacatgtggaaaaattcaagggttccaacaatcagtgttgatagtgacatggtcccaaacatttatttccaagctctggatttctcgcagggccgggtgagctaatgggctttgcctaccggctggagagatttgtgggccttccccttcctactcacttttcctcctcccactcttctcccctcttcaggcccgttcttcctacttttcctcaactctccttcctccctcctccctccttctcccttagctccccccgccccttccccccaccctccattcctctgtctctccattcctccacctactcctcccaatttctcctcttacagccccccttctcaccaccacttataatggcacacagggctccctctgaggtggacggcccggtacgttttcgtagtcggtctagcgtgaaatgattcggtgtaaccggattcttgggaaaggacaggttacgtaaacatttactgatgtggagtaactatactatttgtactctgatatgcaggaaggaaagtgttgattaacacagaagaaaaaaaaaacgaaattagctgattgcatgctcataagtgcaagctgtcattgaactgcttttaaaatagacacagctgggtgtttgtgttgaaaaatttcttgtgtttttgctaatactgaaataagcatgattttgtataccaaacattttcagatcattaaaatttgtaatctgtgctggttttaaacacaaagaaaagaaacagaatcatttcgatgtaaaatttgctgatattatattaactcaatcaggtctgtacaaattgtgcattgtgcctcctaaaatctgtttgatcaatttaatacacaaagtgccaaacgtgggtattgtttgttaacattggacattttattcaaatccgagctcagcagcaaactggaggagctttccttcaatacaacaccagctctgactgatgaatttttaatgttttaaggtgtttcttaggtcaaatctgagcgtgcattgttttgtaatcctgtataaaattttagcaggtggaaataagtttttcttctgtttactttcaaatagcaaaaactggggtttgctaatgagcactgtaataatctgattttatatgctaggagctcatattgactgggaaaatgtttttcattcaaattaaggctatgttatatagtttaaggattgcatgaatgcaagtctctcaaaataggatattcttatgggcataaatagtatatagtttaatgtaatatttaaaaatctattcttcctaatgacaaaagttatttgtaatctggcaagaagtgagttttaattccttttaataatttcaattgcactttatgtcgagatggtgatatatatatatactcacacacatatatatgtgtgtgtgtgtatatatgtatatatatatagcttgttcaacaaaacatgcactgtttactcagcaccccgtgtttgtctcagcaatagcttttttaaagaactgctactatttgaaatggagggggaggggggtcctggacagagtattgtgcaagttgaaagtctctggatggggctttgtatatcctaccagccaatttgggtgcaaattggatttgaaggcctgcctctgtccacctcgggcctcttccctaagcactggaagcccaatcttcagccccactcagtcaccgtgattaccttcagaagacagagggaattcagttgtctcagacctgaaacaaattctctatcattctaatttttttctcatgatggatctaaccttgatctctcacacagacatctacaaatgtgtactattaatgaataaaagcagaagtcaaagtaaatgtaattttctggtatatatcacagtaaacttctctcttgctctgtctgcagtcccccagctctcccaaagaggtcaggccccgtaaagagtgagcagttccaatggaagaattggttaggaaatgaattagattattccttctggagttttaattaataaccaaggctacacagcatacttcatccgagtagggagcagaacatttcatttttccatttggcggtgttatttaagtattctttagggcctctttcctgttttcagtcagtgatccgcctcttttcttcttgctagctgctggttaaaggaaggtgggccccatttacctaaaatccactctgctctccattccctggacagggtgtccttggagtggcacccagcgccaagctctagctaaagttctcttcctgcagatgcacagagaatttctgaccttgtgacgtttgggagtgaggagatcccatacagaggcatccaggtatttccagagatcctgtggcaggtgaggtctgccctcgctggagccaactcgtctataatatcttcctaacagcaggtaatggtgctaacatttagtgattagaagttactctttagcgtgttgtatcctcgttctgtgggttttggttacaggttatacgattaggtttgcgagaacagtagtgaactaaacttctcagtaagaggtcttcacaccagggaagtgttacttttctgcagcagccctgcctcgtcactcattagggagctgcaggttgtcagtggctgctctcagcgcgcgcacgcgcaccctcaacgtccccgggggcgcgcacattcgcgcgtgtgtgtacacacacacacatacacacacacacacacacacagcactaaaactaaattaagaagccgatgattaagacaagcgtctactaggcctgtcatttcagcatccttccatcataacattgtggcaccggcaattttgcttgagaacctcgagtaaaaaaatctatgggagagcctcgtctatgagagtggaaatgtttagcttgtgtaagctagggagccactttaaaagggagaaagtccaaggcgaagaagaggagcaggaggaggaagaggacaaggatgaggagaaggccttgacagagacccctgggactctccaggcaagtctctgtgacgctgccaggtgttccaggctctctgaagccaagtacgtacgcagccaaaagaaaagtcccgtgatcttcagcaaaatgtgcatgggagaagctttgtcccgacattagccaagggagagccaggcgtggaaagatccatgacgaaataccaccagagggccaagggactttagtctctggaaaaacttctcagtcgaatactctgtacatctcttgtaaacattgtaaacataatgtccactacccttttacttgtttcgcgttctttgccagtgccctccagagggtgagtgatttgctgaaggacttgagtccctgatagtgtgagggcggacctgttccatttttagacctgatccagttgtgagaaatggtcccacacagcctggggaagtgcccctgagaggccgattcctatttttcctctagttacccaaggaaatggtgtagtcagtctttcaaaggagataggggaagctccacaaacaaccataacacagcacaacaaaacaaaacaaagagctgaaggggtgttggtgaactagaatgaaggaggctagagctcaggttcttagttttaacccccagctctatcccataaggaagaaagtgagacttggcaaaagccagcccacaaacaatattacctcggattttccatcagtggggcaccaaaacaggaaaaaagaaaataactatatatatctatatatatatatatatctatatatagatatatctatatatcaagctgaaagaaaaacttacagggctaaaaatgctgaactcttaagagctggaactcctaactatcccatcattgttcttacttagttggagcttcaatgtgatttctagtagaagcctcaatcttcttctggaagtgttacaggaccttttagagaaggggtgtgtgtgtgtgtatgtgggggtgggtggcgggtgaaccaagacttctcttttgaagcacacctccaaatggtcccccaaattccagttgtgtgagaaaagatggaaaggtctgactccttcgcaatttttagccggtcaccttcgaactagtcaccgctcaatatttaaatgtacacggaaaacattgaacagttcaaggtccgtatatgagattaataagtatcttataggaaatgcttgcacccactaaaggtctgaaggaatgagaggagagaaaacttcatgagtggggtaagcaagccaggaagcacgcagagaacccttcaccccaaagtctccctttgcgtggggaaagtagtatctactaatcagatctatctgcttctttcaagaaacctgcgtggacaaaccaatgggcaagtttttactttgtgctcatcttacacccacctggccctcactctccggagtcggttccagtaggtcttcctaccccaaccctcgctcctcccccgcgccccttctccaacacgaggccatggaaaatctgttggaggagcgtttctcaagaggtccagcttttctccctcccctgccttgcctcttgaggctgtaactgcgagggttcccttcgaggagcttcgcggagctggggctcccaggtctgcgcgccgctgctccctgcgcatacccgcgagcttgcccgacgctcctttgttgcagacgcttgccgaacgcaatcgagccagaggccttgattctaggcgtcatctctctagatgtccccccttaaagttgccttttcagcctaggtatctctttttttggcacataagaccggatacacaccaaatcaattttgggctaagaggtcatctgccacggtaaacgcggggatatagaaaacagttaaaggtttcaagtctgtattgctataaagcacatttcagagttaaaagactggggtgcatgcaggtgacgttaattcgaatattcaattacatgtaaggggtattgaacccagtgggggataacagtaatacccgtatttctctctcgctctttggaaggagtcgcctgcggggaggagaggagaagacagactaagctgcgcgtagagcggcatcaggagcaagttaccgttagcatgtgtaaacaaaacaactcgactcctctgtgtcagaatcaacaacatcaaaggtgtctgtggggggtcacttttatttttaaaacaatcctagattcttgttcggcaattttagtgtcttccttctgggtttgtcctaaggcacgtaggagagaaagtaatgttcgagaaaaaagctgggtcggcaagtttggctccggagcctatgaactatgaatcaccgcggtgaagaggttttcttttttctttcttttttttttttctcaccctcttcttttctcccggagcgtgaacttgttagataagatagatgaggctacaaggaaacagaaacgctgaaaaagcccagctcctttggagagcagcctgtggggctggcaggcggctgccgcgcgccctcccccagctgaggggcaccggggtagctccggagctgtcccaacttgggcctcctgggccccccgcggggccagacatcacgctcagatcgctgccttggccatcttaccttttggccatagaataaacaccccgaaagacgagagaaaggcctctatttgagctgagtgtctgctctcccgctcgaaacacaaaacagggcccgcaggttttcctagaggaagtcccaatcacaaaacagtcggacagtgggagacgctagagaattctggaattacacatgaaacattattttcctaattaattaaaaataattctcttttcctatttatatgcatacacacgctactgattacacgtccctatttacttgaaaaaatgcttggagtatctttagtacacaatgctcattctttgtccccttgaacatttacagctgataatgtggctggttgggatcaattagcactggattttgccccaggattgctccccagggcggacaagtggagccaactcatctccagcgacttttacttcgctcacgtgcatatccacgccagggctgcagaagcatttcaaaatccgcagccttggtttcacttgattttgttgttgttattgataaatattgggtcatacacaagccacttgtcagagcctttggctccacctcgcagctgcagctcctagcgggacgtttgagtcttgtcctaggaactggaaggatttactgggaaaagaattacacattctctgctccttcagttttggagatggtcccttaagatcatatcatatcgtctgtctgcttgctgtcacatttatttcttgattgttctgatatgtgaacaatgggaaggtgtgtgcatcttttccccctatctttttaaaacaattattgaaatggtttaaaagtgggggaggggagaaagtattgatcccagttctttccgtatatttggaaccaaaagaacataggcaaatagataattgttttaattgtcggggtggggggtgggttggtaggtaggacctttggggtttgtgtaattggagtaataaatacagtttatcttttagctccgccgtatatattctttcccttgatacatattgcacatgaaatgaacccatggtctacttgtcttagcatgtttgaagccccagacacccaacagtatcattacaggaaaaagacagagcccccaaagactgcatgcaggcaggaaggtgactgcctttggggccaatggggaatttatcttgacgttgaacttttctgggaagtgcacccactcgctcaggctcccttgctgcgcagagttcaggtcttccccatgctgaagagcagggaggaggtgtggaaaaaaacagctgatttttgacatttcaaagggactctttctatttatcagtgttaccgaagtgtacgttactggagaaacaacaaaacaagcatagagctgcaattttcaagaaaataggatccccaaatataaaaataaaaaagtagcagataagatgtataattcaatcaaattacacaaaattgcttgaaaatctagaacagtagcgatccattatgcctctgttttattagtgacaagtacaattactctaaaataaagaacactggtgaatatataattggctgtaatcatgttagtgtttgaataaaaatgacgggaacatttaacactttgacaacaaaatgttcagaaataacttatttattcaaacagtttttattttccaccaagtgaaggagaaacacagtttgcttaggaaaatcaatgacttttccctgcaataaataaatatccaaaagggggaaaaaacagccccacacctctatattattactgttattatttttaaccagcaagaagcttaatcctgtgtttgaagtcagcgctcatctagcggttgtttggtgccaaagtccgccacctagaggaagagctctgcagcccagaaccccttttcccgcggtgacctcgggaatcaaagttaatattcctttgcaaattgctctccaaactaactccccctccctttcctcctcacgtcccctttcgccctacaacatcgtcacaatattttcttagtaattgctctgcacaattgacagaagcgccgcgtctccggcagcgctgtgctctcccaggaaagcgtgagggcgcgttggggaagcaggtcagggaatatttggttctggagagacaatccactttcatccacaatggtctagagcagcgtgacttgggaacttataaagtgacggctgatggaggcatcgccacattccaatgcattttttaaccccgggctttcttagaaaaaacaactgggatccccaggtaccgcggggctattcccgacttttccccagcgcccccactccccaccccaaacgcgaagatctgagatctcggctttatttaatcatctactcagttattggagctcagtgggtccttttattctcctaggtgggttgcttgaagaactgagttacaaaggtattaaagaggaggccgtgtttgcaggggtccccctgtggtcagggaaaccgactcaggagaacccccatgtttttacactgagctgagcctccgagtttcaagaaacttgggcagtgtgcgagagaatttggggttgtactcgcaagcaccaggagtctttttgattttagtgttggtggatcctatcggctcctcacacagaaaagccaacctcactcttcaatctcgagctcatcctctcaagtaattattggggaagggagatgctaaacgagggagcatggggctctgggaagaaatttaacaacagcattcgagcgctcaaattctccttaacaccagtgctgcagattctgctgcgatccccgctctggccctccaggaatgtctccccgccccactcctccaaagttttgttttaattgctttagggcacttagaatattgttcaagttgaggatttcctagaaaggcgaggaagatagagttgatttagttttgaaaaaaaaaagttattaatcataagacttcagttgggtttcatttcttctaattttccttcctgtcccctccttccctccttccctctttccctccttccctctttccctccttccctccttccttccttccttccttctttccctgctttttcttcccttttctctttcctctctctccttctccctttccctctctctctcactatctctatgtgcgtctaactgtatctcatctacctccctaaatgtgagagaaatgaagcctcgtgaaggtgatggaggtgaccattttcattctgacttccagaacctccttacttgtcaattcttagtctcaatgctggagtttgaaagacatctcggaggtgattcttctccctcccagagacgacctcagaaccatggtctaagaagctatttttctgactgtgaaattattttttaaggcagctgaaataaatatatatttttaaaataagcctctgaaactgatgccccggtgcatgcttcctctatggattgaggcgctccctaaattcactccacccaaccccctaggagcttaagtcctcagaaatgttttgttttgttttatgcaaaactagcgcttccactctctactcccaggtctatcattcctctcccgcattatattaacggcgatgttagccttcccctacgtgtttaggctgctaatgccggccacttaacgcaacaaaaatgctcatctgtggagattttcccaaggctagctgcaatgttggctcctttttcttttcttttcagggcacgtccattcctccctgcccccttcttcttttcttgagatggggactggaaaaggggtccaacaactctaagcgagtgtgtgaagtttcccctctttctcttgcctgccaatccgtcaagcggggtggtgaatttttggttttctctagagagcaggactatggaagcttcagcgtagattggttgcctctcttagttaacttgccttcttcttcttcttttttttttcccccagtcatgactttcctttctcctcaccttagtcggtgaaagcagttggggattgccctcacagtccttctgcacccttcctagccccaggcttctgaagaaaactccactggctttcttttgtattgacagggaagctgggttggagatctcctctgtattggattgtgtgtgggtgttgtgtatgcttgtttagttttaataactttatgagggagagtctcctgagcttgttgacatttttcttctatggggagattttctggagcacggaatcaccttaatttagataatgaccgtctattaggcttcttaccctttcaagtaaatttagcctgggaaaatcatctgccctcatgattaatacatacctagtgtccaaggagaagaccatcaggggcaatgcaaactgaaactgacttaaagggtaataaatattacccagtgggttttgacttaaaaagatgaatatcgtcattgttcacagctggaacacgtttacttcttaaaagctggtgaagtgtagacacgagaactgttttcccgactctggctctagcactcttttcctatggatccttaggcaagttacttcatctttccagttttttcttaatctggaaaataagtgacctgcaatcactaatatataaaatatcattcattcatcaaattcccaacaacactaccactaaaatataactttaaaaaccataagaatctttggaaaaaatttttggggaggtggggctgggaagcaggaagaaaatctcattctaacagcagcagcgacatctagtgcccataatgctcaatagtagcagtgaaggtgtgggattgtttactgagtttttggaggggagagccatggaggcttttctaataaatcttgactgtctgtcgggcacacaatagtcttttgataataaaaaaaattctaagctttgattatggtaataagacatagagagacaacggtttttacctcaaatctgcacatagggcaattaaagtttgttatgagtattcttccaatgctttaaattccatgtcaaggaactagttctaaaaagtaattttattttgaggggggttgcaatttatttacctaattttccaaattatcactaaatatgtttctacacaaatacttaacaaaattgaaaaaaatttatttttgatattttcttttatctgaactaaccactttgattggagataattatctagtgaatagcagggaacatactaaaaataacaccatgttttaagtaatcacatatatgccctgtctatattaccttggctgaaaatattttataattcaaagaaatggaattcctatgtctatatggattctgtttcttcttcttacagcaaagattaaatgaactatctgggcacaggaaaaatcttagcaatggtagactgcagagccccaaacttttcagaataacaatttcggcatttgcttggcatttaagatgttagagagagagagagagtagacctcttctcattccctgtgactgtaagctggccatgtgcgtgggtgtagtttgtgcatgcacttgctgaagtgtgtgtttaatttcctaatagcaaagcagctgatgactactggtatatggattcatgttttgtaactttaagggaaaccaattttgtttattctatttttcaccaatatttcctagattttactataaaatttggtcactggcatgctatctagctagttctagaatagtaagataaatatacaagttgttaacacctaggggaatttgaggtagtcacaacacttctcttgaaccttagttttcttatgcacaaaatcagaaagttgaatctgctatcccttataattcaaaagatttttaagtcaataaatcatcaaccttcatttgagaagactctcttgtggtgtcttcattgttatttgcaactcttctctatcactcctgagttctctagttaggcatctcatcagaaatgagaattgatgagccagccctccatcttctctatcttcctgtcttgttctttccctttttctgcttttaaaaataacaaaaatctcagacactctagtcaccatttactaactcattggcatgcactcagctctcccttgctatccatccagaaaaatcataatgtgagataggaccaaactgaacacaaagctgaggcagctgtattcaagttttctaacatgttcccatatcctcatggtgatgattaattattattattattattattattattaggaaccagagccaaagtattctggctgcagatagcccacatttttctctctgcttctgtgtccctgtgaataaatttcttcaggaggatgcagtaaaatgtctccaaattcaatctgactaggaacatcattacacacttttagcaataagagtgttcacagaaggcaaatatcactccgtgatgcaaatatgctcagcatttaatatagacctcagggaagctgaagacacacacacatacccatgcacacacacacacatgcatgcgtgtgtgtgttgtatataaagtgtgtgtgtgtatatacatatacacacatatgtgtataaatatatgtatatggagtatcatatctacattgacctgggttgatagaaacaaacaagtttgttacagggaaaattctaaatatatcaaaggtttctttcttagatgcctttttgttgttccctagaaaacttcttagttcctactacttggacttttatttaaaattccttatgctgtacgaagttcataagacatgttttattctgcagatttttcacagacctcataaacacaggtattattctattttagaagtgaggaggctgacccagacagactaaattacttgcttaagatcactccactaggaagacacagaaaaggagtgtgaatcctggctttggacaggccgcaacttacacttggtcatttttagggttggagccttcaaactgggaaacaaggggattgactgaggctgttcattaaacttcaccaggattccaccgcatatatttgagaaatctcaacttttaaagaattttgggacccctagtctgtgtttgctactcctcattcatgtatgattttctcctcttaacactaacaattttatattctttctacaggaaggataatctttggaaaaaaggttgtactctaaacatttttgttaattatcttgggaactaagattttagagttggagggaatctaagacattttgtgattcagcagctctggagtaagatgattgtccgaatcaatggctcctgagaactgtgtgaaatgggagtccaaattattgtcagttctctggtgttgcttcctacccatctccctggatttttggctttatgcattaatacatttggttacttcattcttgctggccaccctgtggactagttttccaagcggctgcattttcagcagcacactgtggacttagccaagcactttaagctgagagctgttaagccttcctggatctattttctctcaagtagctctctgctgccctcaactggtgttcagccaccagagctagctagccttcagcatttacacgaatccaccgtaccctggcttcaagtcagatatgggagcatcattaaccacactgtttatggcttttatagcatggaaaaatagaacccaggcccattcacgtccacttatgcctcaggttctcactttctacaagtttgcagatgggccatgaaggagaatatctgattttcacttatgtgtgctggagtatttggaagggcttacatgaaaatgtaaactttctctttaattatcaaattgagatacacttcagctgatagaccaaatttttgaacatgttgcagttagtcttaagaaatacgaaacattttgtctttgatgtaaacaagatatagagaatcatatgaaatccatgaattacttgtaaactttctcatttgttcaataaatacatagcgtacacatactgtacgctagacattattattggtgctaagaatatagcattggataatggaggcaaaaacctcttctgcctttattaagctaacttcccagttaaggaaaatagacattaaacaagtaaataggtaaaatacattttaagggaatgtaacatttccatatctatttcctaccccaagtactgtagcgattgcatgtttttgttttgttttgttttgttttgtttgagacggagtctcactctgtcgcccaggctggagtgcagtagcgcgatctcggctcactgcaagctccgcctcccgggttcacgccattctcccgcttcagcctcccgagtagctgggactacaggcgcccgccaccacgcccggctgattttttgtatttttagtagagacgggatttcaccgtgttagccaggatggtctcgatttcctgacctcgtgatccaccccctcggcctcccaaagtgctgggattgcaggcgtgagccacagcgcccggccgcgattccatgtttttaagaacatttgtagtaaagttaaagaggcataactagagtttatacagggaccatgcatgctatataaatattggatgcgttatttattaatgaattcataaatttcttgtgaatatagagtagatatatgaaagtgtttgatgcagatgagtgtttgaacctttcagctgaagagacgttttgtcttttgtttctataccaatctctcctgttgaattgccttgtggttggaatatcactttgtcaccccctgtaatagctgccactaaagtccttgctattgctgctattgacgatggtctctggttattatctttacttttctctttgtttttattctgttctcttactatgtcttttgctttcttctcagataaactctgatagataccactgaccaaatagaataagttttccctttcttatgtggtggaactaccagtcataactagggaggtttaatcacctgattttcttttcactaccaggtctatttgaaatgggtatttgtcctaggtaaaatgcagctgatttggaagaaaccaaaatattaatcaaataataaccaagtggtagtggcaattacagatacacagcttctaagtaatatgtttgtatttaaataaataaaatagtccaaattctgaaaaaaaaaccctataatataaaactgagtttgagttgctgggtgagaagcttcatccttgccgtgaaaaatttttctgtgtggaactttccagttttgtttttatgatgcagacgtcttctgctaggcagacatgacaccatctcagtgaaccatgtaggtcagcacagggttaaaaatatgacgtacctatccttatatgaattagaaatgtttatatttattaacacatttcttgaaatatctttaatttttgtttacttctgattttatatatcacagacttgctgtaaaaaatttctagtatattataaagaagaaaataaacataaatcatctattaaagtaaaacctatattctaaataatttggatcatcttgtgggtatattttgtgttcagccttttgttcattattgttatactgtgagcatttccacataactatttttaaaacctatagttacaaatatctatattaacctatggataatgataaacaatttaaccactgtccattaaccattgcttcacatttaggtggtttctaatttttcattattaaaatgacttgagatatataactttgcacataagctttttctccaactttgatcatttataatagattttttaagggcaaggtttttttttttttttggtggggagggatcacatagtatgaatttgtaataaacatttaccatatactggtgaattgtatttctttaaaatttcattactgtggctgggcttggtggctcacgcctgtaatcccagcactttgggaggccgaggcaggtgtgtcatctggggtcaggagttcaagaccagcctggccaatgcagcaaaaccccaaccctactaaaaatacaaaaattagccaggtgtagggttgcatgcctgtaatcccagctactcaggaggctgaggcaggaggatcccttgaacccaggagacagagggtgcagtgacctgatatagtgccactgcactccagcctgggcaacagagcgagactctggcaacaacaacaacaacaacaacaacaacaacaacaacagcaacaaagattgtattagtgtacatttgcagtgatctataaaagtgactgttttggccgggcacggtggctcacacctgtaatcccagcactttgggaggccgaggcgggtggatcacgaggtcaggagatcgagaccatcctggctaacacagtgaaaacccgtctctactaaaaatagaaaaaattagcctggagtggcggaacgcgcctgtagtcccagctactcggtaggcttaggcaggagaatcacttgaacccgggaggtggaggttgcggtgagccaagattgcaccactgcactccagcctgggcaacagagtgagactctgtctcaaaaaaaaaaaaaaaaaagtatctgtcttaatgaaccctcaccaaattttaaattttcttttgccagtttaaaaggagaaatataatacctataactagctttaatgtacttttctttatgtaccagcaagattgaaagaagtttctacaaatatttattacattttaaatttcatcttttatgatctttctgttgtcttttgtgcattgcttatttgaatttttgtgctatttttattaattttaagggcttttctagataaaaatattaaccgtttgtcatctggctcaaatatcttttatagtttttattggtctttttagcctatttacaatatttagttatacacaaatgtaagttttatgcatattcagatattgaaaatgtaatgagcttgaaacttaaaacctgggtttgaatctatcactttgtgtctatgtgaacttgagtaaattattaaacctctgtaccttatttttatttgatataagatggggttaataatgctaattctctcataatgttttctgaggataaaatgcattaaatttataaggaatatagtttgaagtgtaccaggcaaatggtaagcccaaaattaagattgaacatttttattattgtttacagtattcaaatttacaaatgcaaaatgatttttattactttaatattagtattttacatatctaaaatgagttaaatactaagtaaagttttattaactaaatatcaaatatagctaagaattttgtatacctagttttttaaattatataaatgattaacttgagtattctgaggaaaaatactattatgtcttattcatgtacttaatactagttttattcattcaagagatatttgttgaacttttactacatgccagaaactgtagttgaacaagatagcgaacaaatggatgaaaacctttgcttttctgcaattgacggagaaaccataacaaaatacgttagtatcatgaaaaatataatagagaagaataaaggagataagaagtgcaaaaaaagtgcttgcaatttcaaatatgttagccaggggagatcactgagtgtattagttttctgttggtgcctaacaaattactacacatttaaggcttaaaacaacataaatttatttttttttccagtttctgtgggtcagatcaagagtctagacacaaactacctgggtcctttgctcaaagacttacaaaactgaaatgaaggtattgactgtgctctggttctcatctggaattcaggttgttgatggaatttagatccttgtggttgtaggactgtggcctctgttttcttgctgcctctcaactaaggtcactctcagctcctataggacagatccatccatcataatctgaataatcacccttttgattagctaaagaaaggagtgttggtgatcaattgcatcaaatgttgctgataggccaagtaaaatgagaagagaagattgactggatttagcaatacgaaggtcattggtaatcatgaggagaacagatttgttagaatggtggaggtaacagcctcttgggtgatgattaaaaatgaatgggaagaaaaatttgagatagtgtatagacagtttattgagcaggtttgtggaaagggaagcaagagaagaaacattagctagagaggaggtggagccaagagagggtttttttaatgggagaaaagtatgtttaattgctgatggaaatgatacagtagagaggtaaaagtaaaatctgatggcataggagagaaggggtagaattgctggaggaagtgagatgggatcaagtgaccaagtgagatggttggacttaggtgggagtactgataatcatctttcattccagggggacagcaaagtacatggattctgtaggttgtgggtattctcttctgattacttcagttttctcaatggactaggaagcgagctaaaactgagcatgataatggaagtgcttgaaatatgagtaggaaagattaggcatgaaaacgccatcaagaagagtgggagagtaaaagaccaggcaaatttaatatgattgccagacagtgttatggacatacatgaaatgcatgctcatgaatgtaaatgaaaccagtttacatgctgtgtgttttcctaagtcatgttagtttcagggccttcagaactagcaagacagaaagtaatatgtaaccagggttgtgattcagacaaatgactctgaacaagacaggtaagggaattggggatgcaaaaaggagtcactgcaatgattggtcatggaatttaaggtaaaaaggggggaaaaaggagagaagggggagagaaaagtgaaaagcataaattgaagacctaccaagcactatggtaaattattagaataaagtaatacaagcttttctttaaaactggctttctgatgactctaaggctgtgctgtatagagaggtcaagtgatcctgagctttagctcctcatgagctgcaggtaactcccatcacaatgacttattttgacaaactcaaagcactttcatagctcctatcccttggtacacttatgaagaaagcaagggcaggaactaatatctttgctttttatatagatgtaacatctttattttatatctagatatgcataaagagaggatccctggacacagtttttaatttaatttaatttaatttaattttattttattttattttattttattttatttttgagatggagtcttgctccgtcgccaggctggagtgcagtggcatgatctcagctcactgcaatctccacctcctgggttcaagtgattctcctgcctcagcctcctgagtagctgggactacaggcgcatgccaccacacccagctaatttttgtatttttagtagagatggggtttcaccatgttggccaggatggtcttgatctcttgtactgtgatccacccacctcagcctcccaaagtgctgggattacaggcgtgagccactgcgcccggctgacacagttctataatatacagaggtgaaattattgctaactcaaatcactgtttctctgtgtgactgctttcagcttccttagttctagtcctcagcaactcatgctgccctatggttgttccacattccccactgcaatcttcttctcaggattttagtcagcagccatgcactgtaattttctcttttcccaggcttccttcattgaaaacttcctgtgaagttctcagttctattgagtaacccattaaccactttggtaaacgaattccaacttgacctaagttcttaatcttgttatctgtcttagagtggtagttttcagtatgctcttccctcaacccacagtaagaaacacacatcatgacctaggccacaaatacatgtatctacatacacacatacacagctgaaacaaaaaattttccaaaatacccttattacttgtgatacactctgatagtttctattctattatattctatttaaaaatttactggttgagactcactaagctatttaccaactactaatggttgatggactgcagtttgacaataaaaggggcttttgatgcttataatgcaggtaggctataatcatggctcataacacatttaagtattttacaaaataactcctaattgagacctgataggatatgtgtattttagtagctgaaaagagctggaagttagcagttagtagaaagactatagaacttcaaaagacccaaatcctatacttcattctaatacttaataactttgacattggtttcttattctaccctctctcatgttctgtctatctaaaatggagttcaaaacacctgctacgtgtaaccctgaaatttattctcaagagcaagtgaactgtaagtaatggaggtttataaatagcaaatgtaagactctctaattttatttctggattgggtttatgttggaactctgaaagtcaggcaaaccctccaaagataagaatattccctgtcaccaactgctttgattgatcttaagttgccatatattattcaaatatgttgaattatttgaatattatattgtttcttatgtattcaaataaaatttatcatttatcagaaaaatattttaaagttaaaaaattatctacttgatatgatatggtgtataaagctttttatgcactttaacagaaaatgagtcatggttttttattaggttttgagataaatccacacaccaacctggaaggcatagttctaatacagctgctgtacattttcttcacagcttatttgtaatgtctttagtcattgttaggtctgttagtcagaagtttcacatctataatgagactcacttgtggtcatcatttttagtaattatttaaaaaaatgttgacatacaattttgtgtgttgaaattcagttctattcacagatttctccttctgttcttctgcaaatcaacatggataatctcaaaataacagtgcctctctacactctaaggatttaggttgtgtcatcatattaatattttgcccttttactatttccattaattaatttagccctttaacttttactgatcaatgtggaccaaattttaattgcaacattgctatgaaataataccaaattaaagctgatagtgtcatgctgtgagctagtgagagatattaacatttttgaacatgataagttgatttaaaaccagatggacttgtaaccaacttcagaatagtgttcaaaatctagttgtgaagaattttgaatattacattaatggttcaagtttggggtgtctgataaaatgctggtggcttggaaacatgtagctttacattatctctatcccattatactgttattagaaagttaaaacatgcaatgatgtaaagaaggaattcagtagtgtatctgatgaatatctattaattcccataacggatttaaatattaactttaatatagactgtgcaatatggttgattatttactcattaactaaacatttaatcattatctactctgtacaagatactacacgaggtgatgaagacagagtgttgaataaaatttagttcctactccaatgggactcacagtctggtggaggagatgaacacacagtcatatgtatagcataaaatcagtgccacactggaggtatgtactgggtgggttctatggaagtacagaagaaagccacttaagtcttcagattgtttcatggaagaaatgtcattaaatctctgtcttcaaaaattatcttaaaaatttatcgatattgtacatgaaaacataataataaataatttagaagagcttacgataaaaataacagaactgtttctcttcaacagctgtttggattcccggctgtgaactcttttaaatacctttttaaaattttctttcttttttttttttttttttgagacagggtctcactctatcatccagcttggagtgcagtggtgcgatcacagctcactgcagcctcgacctcatggctcaactgatcctcctacctcagcctcctgattagctggcaccacaggtgcagcccaccatacctggctgattaaaaaaaaattgttgagatagggttttgccatgttgcccaggctagagttttaaatacttcgtttgcatttctttttgtgattccttcatgatactaattatattatacttattatgggtggtttagagttatttatcatttcattacttccacaggttacttttgtgataatagtaataccctaacacattatgagatattatcatccccactctttcatccaccttttacctcccttagttcccaccatttattagctaacctagttttattttattaagactattataatttgtactttgaaattaaacagcaaccaaatctctcaccctttctccatgaattaagtctggaaatttacatttttatgactatgctgagtcatgcattgtgctatgacagaatacattctcttttctcactatcaattgcttaaaaccatgccagagatttaatttttttccatttgtatcgtagccttttacacaactttcttcctttattttctttaggttagccatcaacttttctttctttctgtctctgtcttttttggcttcactatattagcacattattgttttaaagaggtaacatgtctttattgcgtttttcacacattctcccagtgattctcaaatggggaggtcttttctcacatgccttcctcctgacaccatcaatccttcctgccatacaggaatatcctgcattgctttcctgggtgtgacccacacttttttcctgtctctcctcttctttcttctctttattttcttcttttttttcttgtttcgtttcttatttttctttattttctttaatttctttatttattttctttaaaaggtatgtggtaaatacataccatattaccaaatgcttttattctggtgtgtatgtgtgtgtgtatgtgtgtgtatctgtatgcgtgtgattgaactgtgtatatcatttttaagtcaaaaatattttctcaaaaactgtaaggttttggtctattttcttagcattcagtgttggtgataagtttaaactaatcctttctctctctctcttttttttaatagaacctgggtctcactctgtcccctaggctggaatgcagtggtgtgatcatggcccactgcagccttgaactcctggacccaagcaatcctcccatctcagcctcccaagtagctggaactacaggtgtaagccacacctggataactcatttatttttagataacctgtttttaatattggaagcttttatgattatctctgggtaggggtctttacattccacttggcatttttcactttaaattctcatgtctttcaacttggaagaatttgattctatcagttctctgttgatttcttctactccattttctgtattcacgttgtttttgggtttctattactcagatatggggcgtccataccacttctataggtcttttatctgttccctcatgctttctgactctgcatttctgctttacactgtaacagagttctctattttattttctagatctttactgttaattccgtttagtagtttgttctgcattttccagttgttagaaatctgttattattcctcattttttgaaaacttctttcctacattcttcttagttacgtattaattctatttttgtgcatttatgttcttattttagtgctgtctcaggagagataagatgtatcgctaagacctagtatgaatttgagtcagaactataagcagcaacaggttgttttagggttaaagtaacaaactagaaacagtagggccacaatttgcttttttttttttttttttttttgatggagtctcaaatctcgctctgtcactcaggctggagtacagtggtgctatctcagctcattgcaacctctgcctcctgggttcaagcgattctcctgcttagccccccagggtagctaggagtacaggcacacgctaccatgaccagctaatttttaatttttggatttttggatttttagtagagatggggtttcaccatgttgtccaggctggtcttgaactcatgtcctcaagtgatctaccctccttggtctcccaaactgttgggattacaggcgtgagccactgtgtctagcctgctgagataatttctaggtgtcatatcatggagggtcttgtagatacactctactaaggacactggacttttatcttagaggcactgaatgttctaattgtgaaaatagtatgatcttgtaaagaaggtatatttaaagactagaggccaggaggacagttagaagcctgttgaaagaatttatgttagtgataaagatctgatccaaagcagaaagatcagagagaagatgaggcaaaaataatagagatactacagaataaatttccaaaattgagtaaaaatatgaagacaggaaatgctaagaacgccaaataggataaaggcagagaaaaccacatgtagtcacatcacaataaaacttccaaaaaccaaagatgaaaaaaagcaaccttaaaaacagccaacagataatcaaacaatatatattaccttcaaaggagcaattaaaggcaaaataagagtgttacctttcttctcaaaattagtaatgaaatccagaaaataatagaatgataatcctaaaaaagctaaaataaaatacgggacagcttataattaaagcctagtctctctctacttttgcctaatgtactatgtccctgttcattaaggcaatatttcgtcttaatgaaatgtccctgttcattaagaaaatataagaaaatactttattctcttataattttgtggagctgatgttcataaaatgcatcatttaaatagttaattttgtaagatgatgggcttggcagagtaactgagatttaaatccttgctgtttaatctgttctaattggttaagggtaatgcccaatttagaagcaaaaagtaaaattgcagatttgtttttaactcccttcagttctgaagtttaatagtttcaaaaacatctcctttattatacttaccctaagaaagttgactcagattaattgtggcaatatttgataaacataataatgccaatattagatgaaatgaaaataattgtgaatagaaggctgtgtgtaataaggggattataaagacgtaggttcagagcagacctttatttatttacttaataggtgagtactggttctgttgcctagtagagtgcagtggctattcacaggcatgaccatggcgcactacagtcgcactcccaaatactgggactacagatgggcaccaccacacccagctgtagagtggatcttttttttctttttttttcttttttttttttttccccgagacggattctcactctgtcgcccacgctggagtgcagtggcgcgatctccactcactgccagctccgtcttccgggttcacgccattctcctgcctcagccttgtgagtagctgggactacaggcgcccgccaccacgcccagctaacattttgcatttttagtagagatggggtttcaccgtgttagccaggatggtctcgatcttctgacctcgtgatcctcctgccttggcctcccaaagtactgggattacaggtgtgagctaccgcgcccggccaagtggatctttgtaagaactgtgggatagcaaatatagaattagggctgtgcaattttatgtccagatagactattatgaaaataaaattgacctccatgagctctggagttagatttcctgtgtaaaaactccagctcttcaatttaccagctgtgtgaccttggacaagtttcttaatgaatctgcatttcactttctgcatctgtaaaaccttttcaagattgtggcataaatttgtaagcatacttcaaatgttaagcctttattataataatgaaaatttggcaaaacaaagctgatgcattcatagtaattgtgtaatgtatttattaatattttattggattctggtatttggcttaggcaaaggaagtatctgaaaatatttacgttttatgtaatgttaaaagaatgaaaagaaaaacaaaaaaaattatcaatatatgctaagaattttgttgacagtccacatatataattttcttaaccctaaaatgaatttataatgtaggtgttattttttgttattttacagacgggaaaactggcttagagatgttaaatatcctggattattgtcagtttaacaaaataatttcggctatcaaagatcaagtgggataaatttcagaggaagaaactgagctccttgcgtaagtcctagaaaaaacataaaaggtggttaactgcctccatataaagctgattttttaaaaagctctcattttaggtttaggggtacatatatggagttgttatacagataaattgtgtgtcatgggggtttggtgtacagattatttcatcaccctggtaataagcataggacccgataggtagttttctgaccttcttcctcctctcacctcaccctcaagtaggcactggtgtctgctgttctcttctttgtgtccatgtatactcaatgtttagctaattctagcctgtaaatttaatcacaaatctcctgttaacaatgctgtgttgcttccttctttggctatttcaactatctgctgatttcttttctttttactactatttcatttgcagtggagccaaccaatcctaacaaaattgaatagtagataaattataccatttcatctgaagaaatacctgtctcatttttactttttctctcactctatcctctactaaaaactgttttcagataaacttctgaaaccaatcatttataactggatataaattcaaattatttttacttctcagaatattatctcaagctaacttgaattgggtaataaaagacattttactctattaaagaaaagctaggcatttcctgaaatatcaattagtttggaagcaattcacctatatgtcatgtgaaccctaaaataaaataaaaaaatctagactggttatgtatattttcctgttgaaaaagatctagatgattttttaaaatgctacatgttactttctaataaatattttttaaaaagttggtcaccagactgcccctaagcaataagagatttccaaatttttatataaagtggaattatgaaacctaattatgtacaactgctgttctaccatgtttgggctacattggtaggaatacctgttagctgtgcattgtcaaagattgcttatacttggagatatgaatgtttggccactcctagttggagccaggcctatattgatcaaggataatgaaaggaagtactgatgatggtatttctttgacagtttcagttaacaggaagtattagtctgaattagggtgtaacaaagggaacaatgattcagtaagttaaagaagatagaactggttttgtcctttacataacaatccaaattgaatggttcaattctgctccatgaagttattcaggaattaaggttctttccattgattgcagtattatcctctaggactgtcatcttctgcatggcagagacagggtcagctgccattccagctggtgataaggggaaagataacatggaagcatacacacggtcctaagacttaggtccataagggacacctatcacattaaattccatggggaaaagaagtccactatgaaaaggaggctggaatattccatttattcctgcatgcaggaataaaaggagagtatcttttggtagacaactaatactctccactttatggtttgtgctgtttgttgaccctattgaagtttcctgatgatgtgcagtcttttctttgtgaaaaggataaagattgatacggtttctgaatggacccttaaattggacgaataataccacccttatttttctgtagactgtgttttacacttgcctatactgtgaaaaataataatcttttattggataagagtctctccattcttattttcttcaacttatttggtctcgggaaaataggaccaatattaaagctgtcttgtgatttacctatgccttgtgaatctgtttctagtcagtttcacttagaaggaaaagggccagattctacctctaattctgcaaggaaaaatacattcttagtagatcagaatacattttaatctacccttttcctgaattagattaaaaaagctttgcttactaaatagcttacaaagtgtcttcttttgaaataacctgtacagataactttgacagggaatttactagggaaattaaacaaagctgcctttattgttaatgaataattcttcaactacaatttaatattagatatactatatttaaaatgaagagagtaatgttcattcttgtaggatagtgattattttctaattttctattaggaccaaacttatgttttgatgtaatgttgggttttgtatgtcacaagtctccagcagatccagaaaggcccttttcacctgttactatgctcacttactagctgatgtctaagtttctctgctagaaattatttatcttggcttaaggctgtaccttgctaaaacccaattatctgtgaaaaaattaaccttttaaactttataatcattttatattttgttctggctgaatttgttgttgagatttttgaaaatgagctagaagaatccttgaggtggctgtgagagggtccactgactatggaaatgaacacaaaaagatttttgtttaaaagatgaatttaatttgtgaattttctctccattgtaggtcactttgagttctggctgtcttacagaggaaaaaatagagctgatcatgaccattataaaaacgtattatgtgcaaaagatgtacatcattccctgaatattttggtttttacttcaaattagaaaattactgttgttgatttaaataaaggacagcatcaacaaaatcatcatgaattggaaatagtgaaatttaatttttataaatctgtgctaaacattttaatggaaatatatttttaatatatattttaatgaaaaaattttaaaatatatattttaatatatattttaaaaatatattttaatgaaaaatgtagtggtgacaaattcaaacaagtaaaataggatttaagttatatgtgttaataaggatattgatctactgttgatattctgaagctattagcaatcaaatgttaccattaattttcagcccttgagctactacttagttttctactcaccagttctaccttttaaaaaattgcaaacaacatggctgtatgatgagggaaaagctttacccctacttggacaaaaaataggccctgcttgtaagattagttgaatgttcaggtccttgtaagttgtaaagaatgaagtttaaaaatttgtgttaggggccgggagtggtggctcactcctgtaatcccagcactttgggaggccgaggtgggaggatcacgaggtcaagagatcaagaccttcctggtcaacatgctgaaaccccgtctctactaaaaatacaaaaattatctgggcgtggtggcatgcacctgtagtcccagctactcggtaggctgaggcaggagaattgcttcaacccgggaggcagaagttgcagtgagctgagatcacgccactgcactccagcctgggtgacagagtgagactctatcccccccaaaaaaagaaaatgtattaggtacatttttttatactttaaatttgatttaagacattatcttgactaccaataactctactgctgtgaatccttgaaatttaatgtttagatttagatctaatagataactacattgtcaactctgtaaatgcttggccattttattaatgttgctattttctagtgcttgcatatagtagacagtaaataagtattgagtggatgtccaataactctttgtcattttaatttgtaagtaattattaattctttttctttaggatacaggctaaacaagagtcaaaggtaagccaaatgagctggagaacagaggagagattagacaagggcaagtgtgtataaggtggtaaaagagtaccctgagataaatgtaaatggtgtgcaatagaggtttaaaggatagagcgcttgtacacagtttggcaattcaggaatgcctccagtaggatatggcatttgaggttgaacctcgcagggtttgtgtggctgagtggtgaattgtgaggatagtcatggcagagagcacattctataaagattagagcaaattcagatgttttccatctctgaccttgtatggttattttacagcacttgtgtcacacactgttttaattattttgtactgtttatacctctcatgccatccactaaattataaacttcttgagggctatgtgttagtaagtcttgtattttctgctaagtctatgagtgctctgtatacattaatatttttaaaaaattactttatatctttcaagttatcaaagattttatagttttcctaattacgttgaatctagtgattaacaaatgaaatagctccttgaataggctgcacaatcaagcatttctaaaacagcagtttagagctaaggcgctggagttagaattgccatctttaaatactggtttagctgttgactaacagatcttgacaatttgttaaccacagtggactcagttttcttatctataaaaagggataatattcatacatacctaatagagctctgtggtagaatatagattgctataaatactttgctactcttccttttgaaagatggagtctaatattcatccctttgagtctgggctggcatttgtgacactcttaaccagtaaacaataatggaagtgatattgtgagacctttgaggctagatcatatgaagctctgaagcttttgctgatctctttttgaatgctgctcttaaaaatctttctgttggagcctttagcctccacataaggagtttgattactccgaacctttagccatcatataagaagtttgactactctgagactcatgagacatgggatctcatgctggagatccacgcgtggattctctggttaacattactagctgagctaaactttttggccattccaggcaaggcatgagacacgtgtgttaagcctcctggtatcctttataacagcccatccaccaggtgaatagcatctggtgatctcagctgacttcatatgtagaactagtatcacctattataacccaactcaatttcttgacccactttcattaaccataaaataatggttgttgttttaagacaccaagttttgaagtagtttattacatggtaaaaaataactggaacaggctattatgaagattaaatcatttgctgtatataaaacagcacagcgctgggcacatattatgtattctataaaagttattgctattatgatattattacaccataaccacaattataaagtctgcatttgtgtgtgtctgtgtatgtcatgtttgtgtatgttatttgcaagtattgattaaatggacttgaaatgaaagccaataatatgctgcactttcatataagagaagctttaagtcatttttttaaagatagggtcttgctatgttgcctaagctggactcaaactcctgggctcaaacaatcctcccgcctcggcctcctgagtagctgggactaaaggtgcgcagcactatgcctggctgctttaagtcattattctataaagaattcttttcttttttttctttcttttttttttttcaagatggattcttgctctgttgaccaggtcagagtgcagtggcatgatctcagctcactgcaacctccgtctcccgggttcaagcaattctcctgcctcagcctcccgagtattggggactacaggcatgcaccaccatctctgcctaatttttgtatttttagtagagatggggtttcaccatgttgtccaggctgatctcgatctcctgaccttgtgatcca

SEQ ID NO: 18 - DNA, 40195 bp, synthetic construct
  source 1..40195: mol_type = other DNA
cccagtcgtcggggggaaacggcggcggcggcgcgggagcgggacagcggcgcggcgggcggcggcgaccggtgggtgccgaacggggcgcggggggcgcgggcggcagcggcggcactgagcgctccggcagcagcgcagttggaccgcaggctcgggcgctggcaagtcatgtagcaaaagcagcaaccacccctcaggaattagaaaaaaaaaaaaaaaggaaaagaaaaaagaaagaaagaaaaaagaaacaaccaccgccccgggggtgaagctccctcgctcccaactgtacttcttggtctccccccccaccccccgctcccccccccgctgcttcctcctcctcctcctcctctcgcagcagcagtcacagcagcagcagcagcagcccaaagggggggagagccgggcggcgggcgggcgcgtcccggagcggccgcggcgaggctggggagacagcgatcgaggcggctatagccacaattaattttccgagctacaggcgcacactagggctgtaaaaaaatttttggtttaacctttcctaccgctgggccaatcagagctggcggcgtgcgggagcgtctctctcgccgtcgaccaatcggaggggccgggcccacccacccccgccccgcccgccggctgccgccccctcgggcgctcgcctccccctctccgcgggcctcggaccccgaggccgcgcggagccgcgtagggatacgccggcgggagcacgcggtgccgcagcccttccctgcgcccccgccaccccctccaccccgccggctccgctccaccgaaattcccctcctagggccaatagctcacatttccggcttcctcctcattgtactgggtcattttcgtttaagcgtcaatatttgtccttagtgattaaagtttgtatccaattttatttttgaaagcgggggtggtggtggtagtgggaagagtgggagtgcagggagaggatgcgagagggcaggaaatggaggaggtagaggcgaggcactacttttccgagtcggccttgcaggccagtgtctggcatttctgagccagcaggaaaattgtgccctttgtaggtaatagtctagcgcagctttggataaggggggagataaggcccaccgctggcgtgtgtgcgggggtgtcctttacttcgggggaggagggtgtgggagagctttgggagcgatgagatgaggaggaggaagtgggcagaggagcgcggggccaaacaggggttacttactcgattcctgaggcctgggcggcggcgagctccgggctccggcaccgcggggaaaccgggactggggctccgggtgctgctgcggcggccggggccgctcagtagtgggcgcctctgatggccttccgaaatgagaggtgcccagagagaaaacagtaccaaacaatctcgaaataaatcaggtaacacatgctatatacgtgtgtctcttccttttccttctctcctgctccagagcgtcgctcacgcgcggggtgactcgatgtcgttttctcctcgccgctccagcctcagcttctaatccgaattggaagcgggtagctgcgagcgatcaccctggaaatattttcgttttgcttgttgacattggagggaaaataagggtgggcgtctatttctttccggttttttttttaagccttcctattttcactccttttttttcctttccgtttctcaaatgcttttattttgtgcgggcctcattcctgaaataacagtatactgaagccagctcttgccaaccctgagtgctgctggaaagcaccggggagccttgggaacgtggatctcacagtcccagcttcttgggctttggaactgggaaggttcggttggaggttgaagtgaatggggaaattgcgaatcgggggctttaaaaggatcccgtggccaggggcagggctaaggcccagcatttcccagggattggaatttagtggggagcacattgatcaaaggctgtttcatcccacatgggcacgaaacataccacattttacggaggatgtagccccagagaagagggaggtggagaagcataaatgaaaggcaccttttccttcctctagaaggtaaataattataaaatgtaagcatttcttcacttggataaactttcattaagatatctggcagatttggtatttaagaaaacgaaggaaggaaaattggtgcttatgcttgatcctaaagttaaattgtaacataaagaatgcggttgatgaatactcaggataatagggggttattccatcctccccacccccagtcttaagaaagagaaagagagaaaaggttgggggggggggaggaaactggagagaataatagataattacaaatagctagtgaattttaaatatgcatctattaggaatgccacaaaggagctttgaatattggactaacctcaaatgtccttccactgtatgtatgtatctttacctatgtctacatatcttctcactttaaaacaaactgtatttacatgtggaaaaattcaagggttccaacaatcagtgttgatagtgacatggtcccaaacatttatttccaagctctggatttctcgcagggccgggtgagctaatgggctttgcctaccggctggagagatttgtgggccttccccttcctactcacttttcctcctcccactcttctcccctcttcaggcccgttcttcctacttttcctcaactctccttcctccctcctccctccttctcccttagctccccccgccccttccccccaccctccattcctctgtctctccattcctccacctactcctcccaatttctcctcttacagccccccttctcaccaccacttataatggcacacagggctccctctgaggtggacggcccggtacgttttcgtagtcggtctagcgtgaaatgattcggtgtaaccggattcttgggaaaggacaggttacgtaaacatttactgatgtggagtaactatactatttgtactctgatatgcaggaaggaaagtgttgattaacacagaagaaaaaaaaaacgaaattagctgattgcatgctcataagtgcaagctgtcattgaactgcttttaaaatagacacagctgggtgtttgtgttgaaaaatttcttgtgtttttgctaatactgaaataagcatgattttgtataccaaacattttcagatcattaaaatttgtaatctgtgctggttttaaacacaaagaaaagaaacagaatcatttcgatgtaaaatttgctgatattatattaactcaatcaggtctgtacaaattgtgcattgtgcctcctaaaatctgtttgatcaatttaatacacaaagtgccaaacgtgggtattgtttgttaacattggacattttattcaaatccgagctcagcagcaaactggaggagctttccttcaatacaacaccagctctgactgatgaatttttaatgttttaaggtgtttcttaggtcaaatctgagcgtgcattgttttgtaatcctgtataaaattttagcaggtggaaataagtttttcttctgtttactttcaaatagcaaaaactggggtttgctaatgagcactgtaataatctgattttatatgctaggagctcatattgactgggaaaatgtttttcattcaaattaaggctatgttatatagtttaaggattgcatgaatgcaagtctctcaaaataggatattcttatgggcataaatagtatatagtttaatgtaatatttaaaaatctattcttcctaatgacaaaagttatttgtaatctggcaagaagtgagttttaattccttttaataatttcaattgcactttatgtcgagatggtgatatatatatatactcacacacatatatatgtgtgtgtgtgtatatatgtatatatatatagcttgttcaacaaaacatgcactgtttactcagcaccccgtgtttgtctcagcaatagcttttttaaagaactgctactatttgaaatggagggggaggggggtcctggacagagtattgtgcaagttgaaagtctctggatggggctttgtatatcctaccagccaatttgggtgcaaattggatttgaaggcctgcctctgtccacctcgggcctcttccctaagcactggaagcccaatcttcagccccactcagtcaccgtgattaccttcagaagacagagggaattcagttgtctcagacctgaaacaaattctctatcattctaatttttttctcatgatggatctaaccttgatctctcacacagacatctacaaatgtgtactattaatgaataaaagcagaagtcaaagtaaatgtaattttctggtatatatcacagtaaacttctctcttgctctgtctgcagtcccccagctctcccaaagaggtcaggccccgtaaagagtgagcagttccaatggaagaattggttaggaaatgaattagattattccttctggagttttaattaataaccaaggctacacagcatacttcatccgagtagggagcagaacatttcatttttccatttggcggtgttatttaagtattctttagggcctctttcctgttttcagtcagtgatccgcctcttttcttcttgctagctgctggttaaaggaaggtgggccccatttacctaaaatccactctgctctccattccctggacagggtgtccttggagtggcacccagcgccaagctctagctaaagttctcttcctgcagatgcacagagaatttctgaccttgtgacgtttgggagtgaggagatcccatacagaggcatccaggtatttccagagatcctgtggcaggtgaggtctgccctcgctggagccaactcgtctataatatcttcctaacagcaggtaatggtgctaacatttagtgattagaagttactctttagcgtgttgtatcctcgttctgtgggttttggttacaggttatacgattaggtttgcgagaacagtagtgaactaaacttctcagtaagaggtcttcacaccagggaagtgttacttttctgcagcagccctgcctcgtcactcattagggagctgcaggttgtcagtggctgctctcagcgcgcgcacgcgcaccctcaacgtccccgggggcgcgcacattcgcgcgtgtgtgtacacacacacacatacacacacacacacacacacagcactaaaactaaattaagaagccgatgattaagacaagcgtctactaggcctgtcatttcagcatccttccatcataacattgtggcaccggcaattttgcttgagaacctcgagtaaaaaaatctatgggagagcctcgtctatgagagtggaaatgtttagcttgtgtaagctagggagccactttaaaagggagaaagtccaaggcgaagaagaggagcaggaggaggaagaggacaaggatgaggagaaggccttgacagagacccctgggactctccaggcaagtctctgtgacgctgccaggtgttccaggctctctgaagccaagtacgtacgcagccaaaagaaaagtcccgtgatcttcagcaaaatgtgcatgggagaagctttgtcccgacattagccaagggagagccaggcgtggaaagatccatgacgaaataccaccagagggccaagggactttagtctctggaaaaacttctcagtcgaatactctgtacatctcttgtaaacattgtaaacataatgtccactacccttttacttgtttcgcgttctttgccagtgccctccagagggtgagtgatttgctgaaggacttgagtccctgatagtgtgagggcggacctgttccatttttagacctgatccagttgtgagaaatggtcccacacagcctggggaagtgcccctgagaggccgattcctatttttcctctagttacccaaggaaatggtgtagtcagtctttcaaaggagataggggaagctccacaaacaaccataacacagcacaacaaaacaaaacaaagagctgaaggggtgttggtgaactagaatgaaggaggctagagctcaggttcttagttttaacccccagctctatcccataaggaagaaagtgagacttggcaaaagccagcccacaaacaatattacctcggattttccatcagtggggcaccaaaacaggaaaaaagaaaataactatatatatctatatatatatatatatctatatatagatatatctatatatcaagctgaaagaaaaacttacagggctaaaaatgctgaactcttaagagctggaactcctaactatcccatcattgttcttacttagttggagcttcaatgtgatttctagtagaagcctcaatcttcttctggaagtgttacaggaccttttagagaaggggtgtgtgtgtgtgtatgtgggggtgggtggcgggtgaaccaagacttctcttttgaagcacacctccaaatggtcccccaaattccagttgtgtgagaaaagatggaaaggtctgactccttcgcaatttttagccggtcaccttcgaactagtcaccgctcaatatttaaatgtacacggaaaacattgaacagttcaaggtccgtatatgagattaataagtatcttataggaaatgcttgcacccactaaaggtctgaaggaatgagaggagagaaaacttcatgagtggggtaagcaagccaggaagcacgcagagaacccttcaccccaaagtctccctttgcgtggggaaagtagtatctactaatcagatctatctgcttctttcaagaaacctgcgtggacaaaccaatgggcaagtttttactttgtgctcatcttacacccacctggccctcactctccggagtcggttccagtaggtcttcctaccccaaccctcgctcctcccccgcgccccttctccaacacgaggccatggaaaatctgttggaggagcgtttctcaagaggtccagcttttctccctcccctgccttgcctcttgaggctgtaactgcgagggttcccttcgaggagcttcgcggagctggggctcccaggtctgcgcgccgctgctccctgcgcatacccgcgagcttgcccgacgctcctttgttgcagacgcttgccgaacgcaatcgagccagaggccttgattctaggcgtcatctctctagatgtccccccttaaagttgccttttcagcctaggtatctctttttttggcacataagaccggatacacaccaaatcaattttgggctaagaggtcatctgccacggtaaacgcggggatatagaaaacagttaaaggtttcaagtctgtattgctataaagcacatttcagagttaaaagactggggtgcatgcaggtgacgttaattcgaatattcaattacatgtaaggggtattgaacccagtgggggataacagtaatacccgtatttctctctcgctctttggaaggagtcgcctgcggggaggagaggagaagacagactaagctgcgcgtagagcggcatcaggagcaagttaccgttagcatgtgtaaacaaaacaactcgactcctctgtgtcagaatcaacaacatcaaaggtgtctgtggggggtcacttttatttttaaaacaatcctagattcttgttcggcaattttagtgtcttccttctgggtttgtcctaaggcacgtaggagagaaagtaatgttcgagaaaaaagctgggtcggcaagtttggctccggagcctatgaactatgaatcaccgcggtgaagaggttttcttttttctttcttttttttttttctcaccctcttcttttctcccggagcgtgaacttgttagataagatagatgaggctacaaggaaacagaaacgctgaaaaagcccagctcctttggagagcagcctgtggggctggcaggcggctgccgcgcgccctcccccagctgaggggcaccggggtagctccggagctgtcccaacttgggcctcctgggccccccgcggggccagacatcacgctcagatcgctgccttggccatcttaccttttggccatagaataaacaccccgaaagacgagagaaaggcctctatttgagctgagtgtctgctctcccgctcgaaacacaaaacagggcccgcaggttttcctagaggaagtcccaatcacaaaacagtcggacagtgggagacgctagagaattctggaattacacatgaaacattattttcctaattaattaaaaataattctcttttcctatttatatgcatacacacgctactgattacacgtccctatttacttgaaaaaatgcttggagtatctttagtacacaatgctcattctttgtccccttgaacatttacagctgataatgtggctggttgggatcaattagcactggattttgccccaggattgctccccagggcggacaagtggagccaactcatctccagcgacttttacttcgctcacgtgcatatccacgccagggctgcagaagcatttcaaaatccgcagccttggtttcacttgattttgttgttgttattgataaatattgggtcatacacaagccacttgtcagagcctttggctccacctcgcagctgcagctcctagcgggacgtttgagtcttgtcctaggaactggaaggatttactgggaaaagaattacacattctctgctccttcagttttggagatggtcccttaagatcatatcatatcgtctgtctgcttgctgtcacatttatttcttgattgttctgatatgtgaacaatgggaaggtgtgtgcatcttttccccctatctttttaaaacaattattgaaatggtttaaaagtgggggaggggagaaagtattgatcccagttctttccgtatatttggaaccaaaagaacataggcaaatagataattgttttaattgtcggggtggggggtgggttggtaggtaggacctttggggtttgtgtaattggagtaataaatacagtttatcttttagctccgccgtatatattctttcccttgatacatattgcacatgaaatgaacccatggtctacttgtcttagcatgtttgaagccccagacacccaacagtatcattacaggaaaaagacagagcccccaaagactgcatgcaggcaggaaggtgactgcctttggggccaatggggaatttatcttgacgttgaacttttctgggaagtgcacccactcgctcaggctcccttgctgcgcagagttcaggtcttccccatgctgaagagcagggaggaggtgtggaaaaaaacagctgatttttgacatttcaaagggactctttctatttatcagtgttaccgaagtgtacgttactggagaaacaacaaaacaagcatagagctgcaattttcaagaaaataggatccccaaatataaaaataaaaaagtagcagataagatgtataattcaatcaaattacacaaaattgcttgaaaatctagaacagtagcgatccattatgcctctgttttattagtgacaagtacaattactctaaaataaagaacactggtgaatatataattggctgtaatcatgttagtgtttgaataaaaatgacgggaacatttaacactttgacaacaaaatgttcagaaataacttatttattcaaacagtttttattttccaccaagtgaaggagaaacacagtttgcttaggaaaatcaatgacttttccctgcaataaataaatatccaaaagggggaaaaaacagccccacacctctatattattactgttattatttttaaccagcaagaagcttaatcctgtgtttgaagtcagcgctcatctagcggttgtttggtgccaaagtccgccacctagaggaagagctctgcagcccagaaccccttttcccgcggtgacctcgggaatcaaagttaatattcctttgcaaattgctctccaaactaactccccctccctttcctcctcacgtcccctttcgccctacaacatcgtcacaatattttcttagtaattgctctgcacaattgacagaagcgccgcgtctccggcagcgctgtgctctcccaggaaagcgtgagggcgcgttggggaagcaggtcagggaatatttggttctggagagacaatccactttcatccacaatggtctagagcagcgtgacttgggaacttataaagtgacggctgatggaggcatcgccacattccaatgcattttttaaccccgggctttcttagaaaaaacaactgggatccccaggtaccgcggggctattcccgacttttccccagcgcccccactccccaccccaaacgcgaagatctgagatctcggctttatttaatcatctactcagttattggagctcagtgggtccttttattctcctaggtgggttgcttgaagaactgagttacaaaggtattaaagaggaggccgtgtttgcaggggtccccctgtggtcagggaaaccgactcaggagaacccccatgtttttacactgagctgagcctccgagtttcaagaaacttgggcagtgtgcgagagaatttggggttgtactcgcaagcaccaggagtctttttgattttagtgttggtggatcctatcggctcctcacacagaaaagccaacctcactcttcaatctcgagctcatcctctcaagtaattattggggaagggagatgctaaacgagggagcatggggctctgggaagaaatttaacaacagcattcgagcgctcaaattctccttaacaccagtgctgcagattctgctgcgatccccgctctggccctccaggaatgtctccccgccccactcctccaaagttttgttttaattgctttagggcacttagaatattgttcaagttgaggatttcctagaaaggcgaggaagatagagttgatttagttttgaaaaaaaaaagttattaatcataagacttcagttgggtttcatttcttctaattttccttcctgtcccctccttccctccttccctctttccctccttccctctttccctccttccctccttccttccttccttccttctttccctgctttttcttcccttttctctttcctctctctccttctccctttccctctctctctcactatctctatgtgcgtctaactgtatctcatctacctccctaaatgtgagagaaatgaagcctcgtgaaggtgatggaggtgaccattttcattctgacttccagaacctccttacttgtcaattcttagtctcaatgctggagtttgaaagacatctcggaggtgattcttctccctcccagagacgacctcagaaccatggtctaagaagctatttttctgactgtgaaattattttttaaggcagctgaaataaatatatatttttaaaataagcctctgaaactgatgccccggtgcatgcttcctctatggattgaggcgctccctaaattcactccacccaaccccctaggagcttaagtcctcagaaatgttttgttttgttttatgcaaaactagcgcttccactctctactcccaggtctatcattcctctcccgcattatattaacggcgatgttagccttcccctacgtgtttaggctgctaatgccggccacttaacgcaacaaaaatgctcatctgtggagattttcccaaggctagctgcaatgttggctcctttttcttttcttttcagggcacgtccattcctccctgcccccttcttcttttcttgagatggggactggaaaaggggtccaacaactctaagcgagtgtgtgaagtttcccctctttctcttgcctgccaatccgtcaagcggggtggtgaatttttggttttctctagagagcaggactatggaagcttcagcgtagattggttgcctctcttagttaacttgccttcttcttcttcttttttttttcccccagtcatgactttcctttctcctcaccttagtcggtgaaagcagttggggattgccctcacagtccttctgcacccttcctagccccaggcttctgaagaaaactccactggctttcttttgtattgacagggaagctgggttggagatctcctctgtattggattgtgtgtgggtgttgtgtatgcttgtttagttttaataactttatgagggagagtctcctgagcttgttgacatttttcttctatggggagattttctggagcacggaatcaccttaatttagataatgaccgtctattaggcttcttaccctttcaagtaaatttagcctgggaaaatcatctgccctcatgattaatacatacctagtgtccaaggagaagaccatcaggggcaatgcaaactgaaactgacttaaagggtaataaatattacccagtgggttttgacttaaaaagatgaatatcgtcattgttcacagctggaacacgtttacttcttaaaagctggtgaagtgtagacacgagaactgttttcccgactctggctctagcactcttttcctatggatccttaggcaagttacttcatctttccagttttttcttaatctggaaaataagtgacctgcaatcactaatatataaaatatcattcattcatcaaattcccaacaacactaccactaaaatataactttaaaaaccataagaatctttggaaaaaatttttggggaggtggggctgggaagcaggaagaaaatctcattctaacagcagcagcgacatctagtgcccataatgctcaatagtagcagtgaaggtgtgggattgtttactgagtttttggaggggagagccatggaggcttttctaataaatcttgactgtctgtcgggcacacaatagtcttttgataataaaaaaaattctaagctttgattatggtaataagacatagagagacaacggtttttacctcaaatctgcacatagggcaattaaagtttgttatgagtattcttccaatgctttaaattccatgtcaaggaactagttctaaaaagtaattttattttgaggggggttgcaatttatttacctaattttccaaattatcactaaatatgtttctacacaaatacttaacaaaattgaaaaaaatttatttttgatattttcttttatctgaactaaccactttgattggagataattatctagtgaatagcagggaacatactaaaaataacaccatgttttaagtaatcacatatatgccctgtctatattaccttggctgaaaatattttataattcaaagaaatggaattcctatgtctatatggattctgtttcttcttcttacagcaaagattaaatgaactatctgggcacaggaaaaatcttagcaatggtagactgcagagccccaaacttttcagaataacaatttcggcatttgcttggcatttaagatgttagagagagagagagagtagacctcttctcattccctgtgactgtaagctggccatgtgcgtgggtgtagtttgtgcatgcacttgctgaagtgtgtgtttaatttcctaatagcaaagcagctgatgactactggtatatggattcatgttttgtaactttaagggaaaccaattttgtttattctatttttcaccaatatttcctagattttactataaaatttggtcactggcatgctatctagctagttctagaatagtaagataaatatacaagttgttaacacctaggggaatttgaggtagtcacaacacttctcttgaaccttagttttcttatgcacaaaatcagaaagttgaatctgctatcccttataattcaaaagatttttaagtcaataaatcatcaaccttcatttgagaagactctcttgtggtgtcttcattgttatttgcaactcttctctatcactcctgagttctctagttaggcatctcatcagaaatgagaattgatgagccagccctccatcttctctatcttcctgtcttgttctttccctttttctgcttttaaaaataacaaaaatctcagacactctagtcaccatttactaactcattggcatgcactcagctctcccttgctatccatccagaaaaatcataatgtgagataggaccaaactgaacacaaagctgaggcagctgtattcaagttttctaacatgttcccatatcctcatggtgatgattaattattattattattattattattattaggaaccagagccaaagtattctggctgcagatagcccacatttttctctctgcttctgtgtccctgtgaataaatttcttcaggaggatgcagtaaaatgtctccaaattcaatctgactaggaacatcattacacacttttagcaataagagtgttcacagaaggcaaatatcactccgtgatgcaaatatgctcagcatttaatatagacctcagggaagctgaagacacacacacatacccatgcacacacacacacatgcatgcgtgtgtgtgttgtatataaagtgtgtgtgtgtatatacatatacacacatatgtgtataaatatatgtatatggagtatcatatctacattgacctgggttgatagaaacaaacaagtttgttacagggaaaattctaaatatatcaaaggtttctttcttagatgcctttttgttgttccctagaaaacttcttagttcctactacttggacttttatttaaaattccttatgctgtacgaagttcataagacatgttttattctgcagatttttcacagacctcataaacacaggtattattctattttagaagtgaggaggctgacccagacagactaaattacttgcttaagatcactccactaggaagacacagaaaaggagtgtgaatcctggctttggacaggccgcaacttacacttggtcatttttagggttggagccttcaaactgggaaacaaggggattgactgaggctgttcattaaacttcaccaggattccaccgcatatatttgagaaatctcaacttttaaagaattttgggacccctagtctgtgtttgctactcctcattcatgtatgattttctcctcttaacactaacaattttatattctttctacaggaaggataatctttggaaaaaaggttgtactctaaacatttttgttaattatcttgggaactaagattttagagttggagggaatctaagacattttgtgattcagcagctctggagtaagatgattgtccgaatcaatggctcctgagaactgtgtgaaatgggagtccaaattattgtcagttctctggtgttgcttcctacccatctccctggatttttggctttatgcattaatacatttggttacttcattcttgctggccaccctgtggactagttttccaagcggctgcattttcagcagcacactgtggacttagccaagcactttaagctgagagctgttaagccttcctggatctattttctctcaagtagctctctgctgccctcaactggtgttcagccaccagagctagctagccttcagcatttacacgaatccaccgtaccctggcttcaagtcagatatgggagcatcattaaccacactgtttatggcttttatagcatggaaaaatagaacccaggcccattcacgtccacttatgcctcaggttctcactttctacaagtttgcagatgggccatgaaggagaatatctgattttcacttatgtgtgctggagtatttggaagggcttacatgaaaatgtaaactttctctttaattatcaaattgagatacacttcagctgatagaccaaatttttgaacatgttgcagttagtcttaagaaatacgaaacattttgtctttgatgtaaacaagatatagagaatcatatgaaatccatgaattacttgtaaactttctcatttgttcaataaatacatagcgtacacatactgtacgctagacattattattggtgctaagaatatagcattggataatggaggcaaaaacctcttctgcctttattaagctaacttcccagttaaggaaaatagacattaaacaagtaaataggtaaaatacattttaagggaatgtaacatttccatatctatttcctaccccaagtactgtagcgattgcatgtttttgttttgttttgttttgttttgtttgagacggagtctcactctgtcgcccaggctggagtgcagtagcgcgatctcggctcactgcaagctccgcctcccgggttcacgccattctcccgcttcagcctcccgagtagctgggactacaggcgcccgccaccacgcccggctgattttttgtatttttagtagagacgggatttcaccgtgttagccaggatggtctcgatttcctgacctcgtgatccaccccctcggcctcccaaagtgctgggattgcaggcgtgagccacagcgcccggccgcgattccatgtttttaagaacatttgtagtaaagttaaagaggcataactagagtttatacagggaccatgcatgctatataaatattggatgcgttatttattaatgaattcataaatttcttgtgaatatagagtagatatatgaaagtgtttgatgcagatgagtgtttgaacctttcagctgaagagacgttttgtcttttgtttctataccaatctctcctgttgaattgccttgtggttggaatatcactttgtcaccccctgtaatagctgccactaaagtccttgctattgctgctattgacgatggtctctggttattatctttacttttctctttgtttttattctgttctcttactatgtcttttgctttcttctcagataaactctgatagataccactgaccaaatagaataagttttccctttcttatgtggtggaactaccagtcataactagggaggtttaatcacctgattttcttttcactaccaggtctatttgaaatgggtatttgtcctaggtaaaatgcagctgatttggaagaaaccaaaatattaatcaaataataaccaagtggtagtggcaattacagatacacagcttctaagtaatatgtttgtatttaaataaataaaatagtccaaattctgaaaaaaaaaccctataatataaaactgagtttgagttgctgggtgagaagcttcatccttgccgtgaaaaatttttctgtgtggaactttccagttttgtttttatgatgcagacgtcttctgctaggcagacatgacaccatctcagtgaaccatgtaggtcagcacagggttaaaaatatgacgtacctatccttatatgaattagaaatgtttatatttattaacacatttcttgaaatatctttaatttttgtttacttctgattttatatatcacagacttgctgtaaaaaatttctagtatattataaagaagaaaataaacataaatcatctattaaagtaaaacctatattctaaataatttggatcatcttgtgggtatattttgtgttcagccttttgttcattattgttatactgtgagcatttccacataactatttttaaaacctatagttacaaatatctatattaacctatggataatgataaacaatttaaccactgtccattaaccattgcttcacatttaggtggtttctaatttttcattattaaaatgacttgagatatataactttgcacataagctttttctccaactttgatcatttataatagattttttaagggcaaggtttttttttttttttggtggggagggatcacatagtatgaatttgtaataaacatttaccatatactggtgaattgtatttctttaaaatttcattactgtggctgggcttggtggctcacgcctgtaatcccagcactttgggaggccgaggcaggtgtgtcatctggggtcaggagttcaagaccagcctggccaatgcagcaaaaccccaaccctactaaaaatacaaaaattagccaggtgtagggttgcatgcctgtaatcccagctactcaggaggctgaggcaggaggatcccttgaacccaggagacagagggtgcagtgacctgatatagtgccactgcactccagcctgggcaacagagcgagactctggcaacaacaacaacaacaacaacaacaacaacaacaacagcaacaaagattgtattagtgtacatttgcagtgatctataaaagtgactgttttggccgggcacggtggctcacacctgtaatcccagcactttgggaggccgaggcgggtggatcacgaggtcaggagatcgagaccatcctggctaacacagtgaaaacccgtctctactaaaaatagaaaaaattagcctggagtggcggaacgcgcctgtagtcccagctactcggtaggcttaggcaggagaatcacttgaacccgggaggtggaggttgcggtgagccaagattgcaccactgcactccagcctgggcaacagagtgagactctgtctcaaaaaaaaaaaaaaaaaagtatctgtcttaatgaaccctcaccaaattttaaattttcttttgccagtttaaaaggagaaatataatacctataactagctttaatgtacttttctttatgtaccagcaagattgaaagaagtttctacaaatatttattacattttaaatttcatcttttatgatctttctgttgtcttttgtgcattgcttatttgaatttttgtgctatttttattaattttaagggcttttctagataaaaatattaaccgtttgtcatctggctcaaatatcttttatagtttttattggtctttttagcctatttacaatatttagttatacacaaatgtaagttttatgcatattcagatattgaaaatgtaatgagcttgaaacttaaaacctgggtttgaatctatcactttgtgtctatgtgaacttgagtaaattattaaacctctgtaccttatttttatttgatataagatggggttaataatgctaattctctcataatgttttctgaggataaaatgcattaaatttataaggaatatagtttgaagtgtaccaggcaaatggtaagcccaaaattaagattgaacatttttattattgtttacagtattcaaatttacaaatgcaaaatgatttttattactttaatattagtattttacatatctaaaatgagttaaatactaagtaaagttttattaactaaatatcaaatatagctaagaattttgtatacctagttttttaaattatataaatgattaacttgagtattctgaggaaaaatactattatgtcttattcatgtacttaatactagttttattcattcaagagatatttgttgaacttttactacatgccagaaactgtagttgaacaagatagcgaacaaatggatgaaaacctttgcttttctgcaattgacggagaaaccataacaaaatacgttagtatcatgaaaaatataatagagaagaataaaggagataagaagtgcaaaaaaagtgcttgcaatttcaaatatgttagccaggggagatcactgagtgtattagttttctgttggtgcctaacaaattactacacatttaaggcttaaaacaacataaatttatttttttttccagtttctgtgggtcagatcaagagtctagacacaaactacctgggtcctttgctcaaagacttacaaaactgaaatgaaggtattgactgtgctctggttctcatctggaattcaggttgttgatggaatttagatccttgtggttgtaggactgtggcctctgttttcttgctgcctctcaactaaggtcactctcagctcctataggacagatccatccatcataatctgaataatcacccttttgattagctaaagaaaggagtgttggtgatcaattgcatcaaatgttgctgataggccaagtaaaatgagaagagaagattgactggatttagcaatacgaaggtcattggtaatcatgaggagaacagatttgttagaatggtggaggtaacagcctcttgggtgatgattaaaaatgaatgggaagaaaaatttgagatagtgtatagacagtttattgagcaggtttgtggaaagggaagcaagagaagaaacattagctagagaggaggtggagccaagagagggtttttttaatgggagaaaagtatgtttaattgctgatggaaatgatacagtagagaggtaaaagtaaaatctgatggcataggagagaaggggtagaattgctggaggaagtgagatgggatcaagtgaccaagtgagatggttggacttaggtgggagtactgataatcatctttcattccagggggacagcaaagtacatggattctgtaggttgtgggtattctcttctgattacttcagttttctcaatggactaggaagcgagctaaaactgagcatgataatggaagtgcttgaaatatgagtaggaaagattaggcatgaaaacgccatcaagaagagtgggagagtaaaagaccaggcaaatttaatatgattgccagacagtgttatggacatacatgaaatgcatgctcatgaatgtaaatgaaaccagtttacatgctgtgtgttttcctaagtcatgttagtttcagggccttcagaactagcaagacagaaagtaatatgtaaccagggttgtgattcagacaaatgactctgaacaagacaggtaagggaattggggatgcaaaaaggagtcactgcaatgattggtcatggaatttaaggtaaaaaggggggaaaaaggagagaagggggagagaaaagtgaaaagcataaattgaagacctaccaagcactatggtaaattattagaataaagtaatacaagcttttctttaaaactggctttctgatgactctaaggctgtgctgtatagagaggtcaagtgatcctgagctttagctcctcatgagctgcaggtaactcccatcacaatgacttattttgacaaactcaaagcactttcatagctcctatcccttggtacacttatgaagaaagcaagggcaggaactaatatctttgctttttatatagatgtaacatctttattttatatctagatatgcataaagagaggatccctggacacagtttttaatttaatttaatttaatttaattttattttattttattttattttattttatttttgagatggagtcttgctccgtcgccaggctggagtgcagtggcatgatctcagctcactgcaatctccacctcctgggttcaagtgattctcctgcctcagcctcctgagtagctgggactacaggcgcatgccaccacacccagctaatttttgtatttttagtagagatggggtttcaccatgttggccaggatggtcttgatctcttgtactgtgatccacccacctcagcctcccaaagtgctgggattacaggcgtgagccactgcgcccggctgacacagttctataatatacagaggtgaaattattgctaactcaaatcactgtttctctgtgtgactgctttcagcttccttagttctagtcctcagcaactcatgctgccctatggttgttccacattccccactgcaatcttcttctcaggattttagtcagcagccatgcactgtaattttctcttttcccaggcttccttcattgaaaacttcctgtgaagttctcagttctattgagtaacccattaaccactttggtaaacgaattccaacttgacctaagttcttaatcttgttatctgtcttagagtggtagttttcagtatgctcttccctcaacccacagtaagaaacacacatcatgacctaggccacaaatacatgtatctacatacacacatacacagctgaaacaaaaaattttccaaaatacccttattacttgtgatacactctgatagtttctattctattatattctatttaaaaatttactggttgagactcactaagctatttaccaactactaatggttgatggactgcagtttgacaataaaaggggcttttgatgcttataatgcaggtaggctataatcatggctcataacacatttaagtattttacaaaataactcctaattgagacctgataggatatgtgtattttagtagctgaaaagagctggaagttagcagttagtagaaagactatagaacttcaaaagacccaaatcctatacttcattctaatacttaataactttgacattggtttcttattctaccctctctcatgttctgtctatctaaaatggagttcaaaacacctgctacgtgtaaccctgaaatttattctcaagagcaagtgaactgtaagtaatggaggtttataaatagcaaatgtaagactctctaattttatttctggattgggtttatgttggaactctgaaagtcaggcaaaccctccaaagataagaatattccctgtcaccaactgctttgattgatcttaagttgccatatattattcaaatatgttgaattatttgaatattatattgtttcttatgtattcaaataaaatttatcatttatcagaaaaatattttaaagttaaaaaattatctacttgatatgatatggtgtataaagctttttatgcactttaacagaaaatgagtcatggttttttattaggttttgagataaatccacacaccaacctggaaggcatagttctaatacagctgctgtacattttcttcacagcttatttgtaatgtctttagtcattgttaggtctgttagtcagaagtttcacatctataatgagactcacttgtggtcatcatttttagtaattatttaaaaaaatgttgacatacaattttgtgtgttgaaattcagttctattcacagatttctccttctgttcttctgcaaatcaacatggataatctcaaaataacagtgcctctctacactctaaggatttaggttgtgtcatcatattaatattttgcccttttactatttccattaattaatttagccctttaacttttactgatcaatgtggaccaaattttaattgcaacattgctatgaaataataccaaattaaagctgatagtgtcatgctgtgagctagtgagagatattaacatttttgaacatgataagttgatttaaaaccagatggacttgtaaccaacttcagaatagtgttcaaaatctagttgtgaagaattttgaatattacattaatggttcaagtttggggtgtctgataaaatgctggtggcttggaaacatgtagctttacattatctctatcccattatactgttattagaaagttaaaacatgcaatgatgtaaagaaggaattcagtagtgtatctgatgaatatctattaattcccataacggatttaaatattaactttaatatagactgtgcaatatggttgattatttactcattaactaaacatttaatcattatctactctgtacaagatactacacgaggtgatgaagacagagtgttgaataaaatttagttcctactccaatgggactcacagtctggtggaggagatgaacacacagtcatatgtatagcataaaatcagtgccacactggaggtatgtactgggtgggttctatggaagtacagaagaaagccacttaagtcttcagattgtttcatggaagaaatgtcattaaatctctgtcttcaaaaattatcttaaaaatttatcgatattgtacatgaaaacataataataaataatttagaagagcttacgataaaaataacagaactgtttctcttcaacagctgtttggattcccggctgtgaactcttttaaatacctttttaaaattttctttcttttttttttttttttttgagacagggtctcactctatcatccagcttggagtgcagtggtgcgatcacagctcactgcagcctcgacctcatggctcaactgatcctcctacctcagcctcctgattagctggcaccacaggtgcagcccaccatacctggctgattaaaaaaaaattgttgagatagggttttgccatgttgcccaggctagagttttaaatacttcgtttgcatttctttttgtgattccttcatgatactaattatattatacttattatgggtggtttagagttatttatcatttcattacttccacaggttacttttgtgataatagtaataccctaacacattatgagatattatcatccccactctttcatccaccttttacctcccttagttcccaccatttattagctaacctagttttattttattaagactattataatttgtactttgaaattaaacagcaaccaaatctctcaccctttctccatgaattaagtctggaaatttacatttttatgactatgctgagtcatgcattgtgctatgacagaatacattctcttttctcactatcaattgcttaaaaccatgccagagatttaatttttttccatttgtatcgtagccttttacacaactttcttcctttattttctttaggttagccatcaacttttctttctttctgtctctgtcttttttggcttcactatattagcacattattgttttaaagaggtaacatgtctttattgcgtttttcacacattctcccagtgattctcaaatggggaggtcttttctcacatgccttcctcctgacaccatcaatccttcctgccatacaggaatatcctgcattgctttcctgggtgtgacccacacttttttcctgtctctcctcttctttcttctctttattttcttcttttttttcttgtttcgtttcttatttttctttattttctttaatttctttatttattttctttaaaaggtatgtggtaaatacataccatattaccaaatgcttttattctggtgtgtatgtgtgtgtgtatgtgtgtgtatctgtatgcgtgtgattgaactgtgtatatcatttttaagtcaaaaatattttctcaaaaactgtaaggttttggtctattttcttagcattcagtgttggtgataagtttaaactaatcctttctctctctctcttttttttaatagaacctgggtctcactctgtcccctaggctggaatgcagtggtgtgatcatggcccactgcagccttgaactcctggacccaagcaatcctcccatctcagcctcccaagtagctggaactacaggtgtaagccacacctggataactcatttatttttagataacctgtttttaatattggaagcttttatgattatctctgggtaggggtctttacattccacttggcatttttcactttaaattctcatgtctttcaacttggaagaatttgattctatcagttctctgttgatttcttctactccattttctgtattcacgttgtttttgggtttctattactcagatatggggcgtccataccacttctataggtcttttatctgttccctcatgctttctgactctgcatttctgctttacactgtaacagagttctctattttattttctagatctttactgttaattccgtttagtagtttgttctgcattttccagttgttagaaatctgttattattcctcattttttgaaaacttctttcctacattcttcttagttacgtattaattctatttttgtgcatttatgttcttattttagtgctgtctcaggagagataagatgtatcgctaagacctagtatgaatttgagtcagaactataagcagcaacaggttgttttagggttaaagtaacaaactagaaacagtagggccacaatttgcttttttttttttttttttttttgatggagtctcaaatctcgctctgtcactcaggctggagtacagtggtgctatctcagctcattgcaacctctgcctcctgggttcaagcgattctcctgcttagccccccagggtagctaggagtacaggcacacgctaccatgaccagctaatttttaatttttggatttttggatttttagtagagatggggtttcaccatgttgtccaggctggtcttgaactcatgtcctcaagtgatctaccctccttggtctcccaaactgttgggattacaggcgtgagccactgtgtctagcctgctgagataatttctaggtgtcatatcatggagggtcttgtagatacactctactaaggacactggacttttatcttagaggcactgaatgttctaattgtgaaaatagtatgatcttgtaaagaaggtatatttaaagactagaggccaggaggacagttagaagcctgttgaaagaatttatgttagtgataaagatctgatccaaagcagaaagatcagagagaagatgaggcaaaaataatagagatactacagaataaatttccaaaattgagtaaaaatatgaagacaggaaatgctaagaacgccaaataggataaaggcagagaaaaccacatgtagtcacatcacaataaaacttccaaaaaccaaagatgaaaaaaagcaaccttaaaaacagccaacagataatcaaacaatatatattaccttcaaaggagcaattaaaggcaaaataagagtgttacctttcttctcaaaattagtaatgaaatccagaaaataatagaatgataatcctaaaaaagctaaaataaaatacgggacagcttataattaaagcctagtctctctctacttttgcctaatgtactatgtccctgttcattaaggcaatatttcgtcttaatgaaatgtccctgttcattaagaaaatataagaaaatactttattctcttataattttgtggagctgatgttcataaaatgcatcatttaaatagttaattttgtaagatgatgggcttggcagagtaactgagatttaaatccttgctgtttaatctgttctaattggttaagggtaatgcccaatttagaagcaaaaagtaaaattgcagatttgtttttaactcccttcagttctgaagtttaatagtttcaaaaacatctcctttattatacttaccctaagaaagttgactcagattaattgtggcaatatttgataaacataataatgccaatattagatgaaatgaaaataattgtgaatagaaggctgtgtgtaataaggggattataaagacgtaggttcagagcagacctttatttatttacttaataggtgagtactggttctgttgcctagtagagtgcagtggctattcacaggcatgaccatggcgcactacagtcgcactcccaaatactgggactacagatgggcaccaccacacccagctgtagagtggatcttttttttctttttttttcttttttttttttttccccgagacggattctcactctgtcgcccacgctggagtgcagtggcgcgatctccactcactgccagctccgtcttccgggttcacgccattctcctgcctcagccttgtgagtagctgggactacaggcgcccgccaccacgcccagctaacattttgcatttttagtagagatggggtttcaccgtgttagccaggatggtctcgatcttctgacctcgtgatcctcctgccttggcctcccaaagtactgggattacaggtgtgagctaccgcgcccggccaagtggatctttgtaagaactgtgggatagcaaatatagaattagggctgtgcaattttatgtccagatagactattatgaaaataaaattgacctccatgagctctggagttagatttcctgtgtaaaaactccagctcttcaatttaccagctgtgtgaccttggacaagtttcttaatgaatctgcatttcactttctgcatctgtaaaaccttttcaagattgtggcataaatttgtaagcatacttcaaatgttaagcctttattataataatgaaaatttggcaaaacaaagctgatgcattcatagtaattgtgtaatgtatttattaatattttattggattctggtatttggcttaggcaaaggaagtatctgaaaatatttacgttttatgtaatgttaaaagaatgaaaagaaaaacaaaaaaaattatcaatatatgctaagaattttgttgacagtccacatatataattttcttaaccctaaaatgaatttataatgtaggtgttattttttgttattttacagacgggaaaactggcttagagatgttaaatatcctggattattgtcagtttaacaaaataatttcggctatcaaagatcaagtgggataaatttcagaggaagaaactgagctccttgcgtaagtcctagaaaaaacataaaaggtggttaactgcctccatataaagctgattttttaaaaagctctcattttaggtttaggggtacatatatggagttgttatacagataaattgtgtgtcatgggggtttggtgtacagattatttcatcaccctggtaataagcataggacccgataggtagttttctgaccttcttcctcctctcacctcaccctcaagtaggcactggtgtctgctgttctcttctttgtgtccatgtatactcaatgtttagctaattctagcctgtaaatttaatcacaaatctcctgttaacaatgctgtgttgcttccttctttggctatttcaactatctgctgatttcttttctttttactactatttcatttgcagtggagccaaccaatcctaacaaaattgaatagtagataaattataccatttcatctgaagaaatacctgtctcatttttactttttctctcactctatcctctactaaaaactgttttcagataaacttctgaaaccaatcatttataactggatataaattcaaattatttttacttctcagaatattatctcaagctaacttgaattgggtaataaaagacattttactctattaaagaaaagctaggcatttcctgaaatatcaattagtttggaagcaattcacctatatgtcatgtgaaccctaaaataaaataaaaaaatctagactggttatgtatattttcctgttgaaaaagatctagatgattttttaaaatgctacatgttactttctaataaatattttttaaaaagttggtcaccagactgcccctaagcaataagagatttccaaatttttatataaagtggaattatgaaacctaattatgtacaactgctgttctaccatgtttgggctacattggtaggaatacctgttagctgtgcattgtcaaagattgcttatacttggagatatgaatgtttggccactcctagttggagccaggcctatattgatcaaggataatgaaaggaagtactgatgatggtatttctttgacagtttcagttaacaggaagtattagtctgaattagggtgtaacaaagggaacaatgattcagtaagttaaagaagatagaactggttttgtcctttacataacaatccaaattgaatggttcaattctgctccatgaagttattcaggaattaaggttctttccattgattgcagtattatcctctaggactgtcatcttctgcatggcagagacagggtcagctgccattccagctggtgataaggggaaagataacatggaagcatacacacggtcctaagacttaggtccataagggacacctatcacattaaattccatggggaaaagaagtccactatgaaaaggaggctggaatattccatttattcctgcatgcaggaataaaaggagagtatcttttggtagacaactaatactctccactttatggtttgtgctgtttgttgaccctattgaagtttcctgatgatgtgcagtcttttctttgtgaaaaggataaagattgatacggtttctgaatggacccttaaattggacgaataataccacccttatttttctgtagactgtgttttacacttgcctatactgtgaaaaataataatcttttattggataagagtctctccattcttattttcttcaacttatttggtctcgggaaaataggaccaatattaaagctgtcttgtgatttacctatgccttgtgaatctgtttctagtcagtttcacttagaaggaaaagggccagattctacctctaattctgcaaggaaaaatacattcttagtagatcagaatacattttaatctacccttttcctgaattagattaaaaaagctttgcttactaaatagcttacaaagtgtcttcttttgaaataacctgtacagataactttgacagggaatttactagggaaattaaacaaagctgcctttattgttaatgaataattcttcaactacaatttaatattagatatactatatttaaaatgaagagagtaatgttcattcttgtaggatagtgattattttctaattttctattaggaccaaacttatgttttgatgtaatgttgggttttgtatgtcacaagtctccagcagatccagaaaggcccttttcacctgttactatgctcacttactagctgatgtctaagtttctctgctagaaattatttatcttggcttaaggctgtaccttgctaaaacccaattatctgtgaaaaaattaaccttttaaactttataatcattttatattttgttctggctgaatttgttgttgagatttttgaaaatgagctagaagaatccttgaggtggctgtgagagggtccactgactatggaaatgaacacaaaaagatttttgtttaaaagatgaatttaatttgtgaattttctctccattgtaggtcactttgagttctggctgtcttacagaggaaaaaatagagctgatcatgaccattataaaaacgtattatgtgcaaaagatgtacatcattccctgaatattttggtttttacttcaaattagaaaattactgttgttgatttaaataaaggacagcatcaacaaaatcatcatgaattggaaatagtgaaatttaatttttataaatctgtgctaaacattttaatggaaatatatttttaatatatattttaatgaaaaaattttaaaatatatattttaatatatattttaaaaatatattttaatgaaaaatgtagtggtgacaaattcaaacaagtaaaataggatttaagttatatgtgttaataaggatattgatctactgttgatattctgaagctattagcaatcaaatgttaccattaattttcagcccttgagctactacttagttttctactcaccagttctaccttttaaaaaattgcaaacaacatggctgtatgatgagggaaaagctttacccctacttggacaaaaaataggccctgcttgtaagattagttgaatgttcaggtccttgtaagttgtaaagaatgaagtttaaaaatttgtgttaggggccgggagtggtggctcactcctgtaatcccagcactttgggaggccgaggtgggaggatcacgaggtcaagagatcaagaccttcctggtcaacatgctgaaaccccgtctctactaaaaatacaaaaattatctgggcgtggtggcatgcacctgtagtcccagctactcggtaggctgaggcaggagaattgcttcaacccgggaggcagaagttgcagtgagctgagatcacgccactgcactccagcctgggtgacagagtgagactctatcccccccaaaaaaagaaaatgtattaggtacatttttttatactttaaatttgatttaagacattatcttgactaccaataactctactgctgtgaatccttgaaatttaatgtttagatttagatctaatagataactacattgtcaactctgtaaatgcttggccattttattaatgttgctattttctagtgcttgcatatagtagacagtaaataagtattgagtggatgtccaataactctttgtcattttaatttgtaagtaattattaattctttttctttaggatacaggctaaacaagagtcaaaggtaagccaaatgagctggagaacagaggagagattagacaagggcaagtgtgtataaggtggtaaaagagtaccctgagataaatgtaaatggtgtgcaatagaggtttaaaggatagagcgcttgtacacagtttggcaattcaggaatgcctccagtaggatatggcatttgaggttgaacctcgcagggtttgtgtggctgagtggtgaattgtgaggatagtcatggcagagagcacattctataaagattagagcaaattcagatgttttccatctctgaccttgtatggttattttacagcacttgtgtcacacactgttttaattattttgtactgtttatacctctcatgccatccactaaattataaacttcttgagggctatgtgttagtaagtcttgtattttctgctaagtctatgagtgctctgtatacattaatatttttaaaaaattactttatatctttcaagttatcaaagattttatagttttcctaattacgttgaatctagtgattaacaaatgaaatagctccttgaataggctgcacaatcaagcatttctaaaacagcagtttagagctaaggcgctggagttagaattgccatctttaaatactggtttagctgttgactaacagatcttgacaatttgttaaccacagtggactcagttttcttatctataaaaagggataatattcatacatacctaatagagctctgtggtagaatatagattgctataaatactttgctactcttccttttgaaagatggagtctaatattcatccctttgagtctgggctggcatttgtgacactcttaaccagtaaacaataatggaagtgatattgtgagacctttgaggctagatcatatgaagctctgaagcttttgctgatctctttttgaatgctgctcttaaaaatctttctgttggagcctttagcctccacataaggagtttgattactccgaacctttagccatcatataagaagtttgactactctgagactcatgagacatgggatctcatgctggagatccacgcgtggattctctggttaacattactagctgagctaaactttttggccattccaggcaaggcatgagacacgtgtgttaagcctcctggtatcctttataacagcccatccaccaggtgaatagcatctggtgatctcagctgacttcatatgtagaactagtatcacctattataacccaactcaatttcttgacccactttcattaaccataaaataatggttgttgttttaagacaccaagttttgaagtagtttattacatggtaaaaaataactggaacaggctattatgaagattaaatcatttgctgtatataaaacagcacagcgctgggcacatattatgtattctataaaagttattgctattatgatattattacaccataaccacaattataaagtctgcatttgtgtgtgtctgtgtatgtcatgtttgtgtatgttatttgcaagtattgattaaatggacttgaaatgaaagccaataatatgctgcactttcatataagagaagctttaagtcatttttttaaagatagggtcttgctatgttgcctaagctggactcaaactcctgggctcaaacaatcctcccgcctcggcctcctgagtagctgggactaaaggtgcgcagcactatgcctggctgctttaagtcattattctataaagaattcttttcttttttttctttcttttttttttttcaagatggattcttgctctgttgaccaggtcagagtgcagtggcatgatctcagctcactgcaacctccgtctcccgggttcaagcaattctcctgcctcagcctcccgagtattggggactacaggcatgcaccaccatctctgcctaatttttgtatttttagtagagatggggtttcaccatgttgtccaggctgatctcgatctcctgaccttgtgatccacccgccttggcctcccatcgtgctgggatcataggcgtgagccagaattgttatctttccctgactaagaattaaatgtttttcttcttcaggttttttctttttttcttctttttttttttggtcttagtgccttctcatgtcactctatactgcccatctgaacaggccatctgactgatcttcactacttccttcgtttcagttaccataaattcttaatgaattctaaatatttacctttagaccaacttatttttgaatttcagatgtgaacattcaactgcttactgaaaatccccatcttaacgtcccacaggaacctccagaccaacatagtcaaaactgaaccccttagctccttatcaaatttccttattttaattcactatccattatttgtgtaagtcaaaacctaggtttttaacaagaagtctggcttaacatcacaaatcacattggcacatgttcattaaaataggatctggtctaggtaattgcaaagtttaaggctttcataatgtagcttatatatcatgatactgaactgtgttttatgaagcaataatacattataaataatgcagaaaattttataaagtgcctgatttttgggatatgctaagcaaactacaaaagtttggcacatcttgggatttaagccttttctatctattatttttcctgctctagcttattgaaacttgtgactagaaccttcccataaattttaatctgaagatatcaaatagccttatagatttaatttgtttgcatgtttcttaatgctttgaaacccgtgaagtaatcaagactattgctttttctagttggcgcatagcaccttttgtctcctctccatagcattgtcaagaaagacattttattctacgccagccatttatgtccagtcccactagtactcaaaatcttgaaattttaaattaaatttaatcaggctgttgtacagaattatttccactatcctaatgtcattatgcttcatatcattttaatttatatttaagtacaatttttgtttgtaatttttggtgctctgatctttaatacctggaaataattttagcaaaaatttatgtggtttacatttgggaaatgctttatctttctgttgaagattcacgatacatgctaacatacgtaaactctgaaaagtaaaagcttgtggaaaattatacttgttcaggttgtttaatccagcagttcccatggaatgtaaaaaatgaataacatttgtcatactatttactatttagtttgagagacctgcaactttaaaattgatactcagcttcgtgaataccttttctacttcagggactacataagagaggaaatctttcatttgtaaacattctactttgtagataatatgggagtctttttccttttcttaaatggcttccctcattgctcaccattgcgtcagatgacttccaaaattccttgctaatcagttttaggtgggcttgctttctgaaggtgagtttctctctttcccctgtccctatccttacattgccaagtctgtgtgtcctctttcttctgcctttttgctttcttcctttcctttctgacattctagattttgtcttgctgatattctttcagctttatagaatattattatttttgtgctaccacaatgattttacaagaaaacacgcgttaagggatagctgtttacctcgggatgcttcagttaacaaagtattttacgtagatttcctttagtctgaaatctctgtacttcagattagttccatcccctgacttttcagaaacacatcagttgtattaaaacgaagcatatctgtaacatgcttacaagtatgtgctaatgtaacagcactaagagaagaatagtattttggcagataaatttttaattaattgctgtaataaaatcagctaaaactctatcctcctcacagcatagaacatgtacttcagtttttaggtcagctcctacaagacaattaactggcaaggaagtcagtgctcttcattacatggatacaacaatatcagagaactgttagaataacttaaacttctctaatgagcttcagagctttaatgagaagattttctattcttatgtgattaatattgctctgtgatagaaagagaactttttttttttctgaaaggaaatgtagagcaaaagagttcaagataaatgattcagaggaagttaagaagtggttaatcccttatagcccaaaggggcttaattattgataactagtaactgccaccagcataggtaggctctccaaataatcattatagcctagaataaagtaacaaaaactatttttaaactaccagcattttaacaatttattttctgaattcagtctatcaagcaattagcttagatggctataaaactgtgtgagcttgtgaacctataagtttagagacacttatgattcacgattagtcttgaaaattttttatataaaaatagtcccagtaaatcactaagtcaaaatggacgaactggttcgatgtgtgtgtatgtgtatgtctaggtacacagctgtgtgtgtgtctaggtgtgtagctgtgtgtgtgtccacacacattttgaaaatcaacaggttagcagaaacaggatgacgaaggaagaatgagaaaaagagatagttataaagagtcactgataataagcaaaacattcttaggatatgtgggtgtatgtaatcagtggcctttttgtacaaatacatcttttttgacatattagcttccatcgacatcaaccgaagctgatttcagaatgtcaaggagaactagtgagaagtaaaagtgactgtaaaagtagtggaagtagaagcagtgaagatgtagagaagtttgtgaaagaagtaaaatagacgtagatggtcatcacatttaaataaaggttagtcatgcactttgaacagacactggttaaagatatttaggaaaatagaatggattttaaattttaattggggaagctgattgtaaagaagaaggagtaaaagcaaataccagaataaaaacactagagagggaaactatttcgttattagtttcagctatctccgaatgtgtgtttaggtttcaacttcagcaggagacatcaaatttattcagaagaagagctaaattgatgatggcatatcaattcaaaagctatgctttttgaagctttttgggaaagacattctcctgtgtgctgtcttttcactcccactgggccacataagaatgggcatggaacccttccttaccagtaggtaagggggcccacacaggaagtactgggcttatggccttatgtgggaatatcttttgctgtttcagaatcaacgtgtagttctttgttctgcttaagtgggtgtttcaatagtcctcagacatgtcctctccccacaattttttttcagataccatagaggagaggtcgaggtccttccactgaagcaccaagtggggtatataaagccacactgcccatgagggctgtggtgtgagtctgctagccgtgagagacagatgcattacaaagggatggatttagtgcactctttcctctcttactgtgagtagatgctgcacttcttgaacctggtgggagtgtgtgttgtctgttctcagagaccttgaatcatacacagtttttttttcacccccgggtgacatttacctccttttaacattggctacactcgtgtaatattctgtcctgcagcacagcctgaacagccagtgaacaatgaaacaaaacttcttcccaagcctcaatgaggattgtgccatagtttgaaggtaggagaatttcaagccattaatcgtctatgaatactgaataccagtgagaatggctccttcaggtctatcagcttattgctgcttctgtgttattcttaggccaaagaacaatggtcaatccagcaatcaactcattttggctgatttgtgcctttagtccaggcttctcagttgtaccacacagccgttcttctcttcaatttcagggtcattccttcaggaattctgtcaggaataagcagcaagtttcctactgctctgggtcctccttagacccttcttatttcatgttcagaggaatgaactccacatcctgtcctaaactccaccttccttctattattttttcatatttgacattgatactcattgaaactagaatctgaattattaccaccaaagatattacctttttttttgcaccggagagcttaatctttaaaatgttctgtgagagatgggaatggtgtactatctttctagtgtggcaaaagaagagagagtaacaaaataattgtgttcatattaagattatttgaatataaatattttgccaaatttgtatttaggtgtccatgttatttttaataattggattgatataagtagatcttaagaaatatcaaaaaaaaccgttttgtcaaattggagtcaaggaggcatggaaattctcttcatttgcagtactcctaaattcctgcttattctgtggttgaatttatatcaacaaagagaattggagattaaggcaattaatcagaaattttaattaaaaaaggaatggaaaaaataagtaaaatttcctatagaattctagggtttgacatctacacagatatattcattaggtagaataataagtcttttctttgctcttatgaaaatattgttctctaattgtcaatgagcataaagtaaaatgaagatagattatacaaatatccttattgattaaaagtttaaaattgccagtgcccaagaaatatgcctgaattcaaggatatcttattgcaaagaatatcttactgcagtttgttttttttccttactgtaaataaagggtcaaccaacagctaataactcacaactgcatcttttcttgttagaacatgtcgcccagcactttttgattagatgttctcagataatagctggggcagattgaccaaaggagagcaattttttttttaatcaggataacataatgattccatcacaaactttagaaagggctatggatatcaccaattttctttttttgtctattattagtgctgtatctttagtggaattgaagacatgaccttctgcaggtttcaatacaactttataaaaggctgtaacaatgcctttaaaatcaaatctattaactgttctacagagaagccagttttctagtaacttggccataacttaaagacagggaccctgaaaacaccaagcatctgcctgcaatctgaattatctccctgactagattcagtaatcttggctcttccctgcatttaagtcattttgtgtacatcaattttctcacctatgaaagaagagagttgtccaacatgttctgatgttctttcagttctgagttctttggataatttgattggagcccaagagatgtcctaaatctgactggcatgcagtaaatgtttgttgattgtgaaaaaatatttgacttccaaatgggttgtaaaatatctcgtgtggacatactgcagcaagttaacacttataattttgggagcagaataaatgtaattaagtcacaaatgcaaatgaattctctgcttatttaattcaaagagaaatagttgtctaaaaacaatctgacccatatactggttgaaaatagtaagcattatttttttatgaagggccaagtaagtggcttacatatattaaatgtaataaatgtattcaaaaagattttcacctagttgaactgcatttatacttcatgaatatcactagtgtaatcattactttaaaaacatatagctggagaaataagcatttagacaggtaagaaaacagactgaagttttaaattttatttttaacagtcattctaaaagaattatttatc

SEQ ID NO: 19 - DNA, 8117 bp, synthetic construct
  source 1..8117: mol_type = other DNA
taaattcttaatgaattctaaatatttacctttagaccaacttatttttgaatttcagatgtgaacattcaactgcttactgaaaatccccatcttaacgtcccacaggaacctccagaccaacatagtcaaaactgaaccccttagctccttatcaaatttccttattttaattcactatccattatttgtgtaagtcaaaacctaggtttttaacaagaagtctggcttaacatcacaaatcacattggcacatgttcattaaaataggatctggtctaggtaattgcaaagtttaaggctttcataatgtagcttatatatcatgatactgaactgtgttttatgaagcaataatacattataaataatgcagaaaattttataaagtgcctgatttttgggatatgctaagcaaactacaaaagtttggcacatcttgggatttaagccttttctatctattatttttcctgctctagcttattgaaacttgtgactagaaccttcccataaattttaatctgaagatatcaaatagccttatagatttaatttgtttgcatgtttcttaatgctttgaaacccgtgaagtaatcaagactattgctttttctagttggcgcatagcaccttttgtctcctctccatagcattgtcaagaaagacattttattctacgccagccatttatgtccagtcccactagtactcaaaatcttgaaattttaaattaaatttaatcaggctgttgtacagaattatttccactatcctaatgtcattatgcttcatatcattttaatttatatttaagtacaatttttgtttgtaatttttggtgctctgatctttaatacctggaaataattttagcaaaaatttatgtggtttacatttgggaaatgctttatctttctgttgaagattcacgatacatgctaacatacgtaaactctgaaaagtaaaagcttgtggaaaattatacttgttcaggttgtttaatccagcagttcccatggaatgtaaaaaatgaataacatttgtcatactatttactatttagtttgagagacctgcaactttaaaattgatactcagcttcgtgaataccttttctacttcagggactacataagagaggaaatctttcatttgtaaacattctactttgtagataatatgggagtctttttccttttcttaaatggcttccctcattgctcaccattgcgtcagatgacttccaaaattccttgctaatcagttttaggtgggcttgctttctgaaggtgagtttctctctttcccctgtccctatccttacattgccaagtctgtgtgtcctctttcttctgcctttttgctttcttcctttcctttctgacattctagattttgtcttgctgatattctttcagctttatagaatattattatttttgtgctaccacaatgattttacaagaaaacacgcgttaagggatagctgtttacctcgggatgcttcagttaacaaagtattttacgtagatttcctttagtctgaaatctctgtacttcagattagttccatcccctgacttttcagaaacacatcagttgtattaaaacgaagcatatctgtaacatgcttacaagtatgtgctaatgtaacagcactaagagaagaatagtattttggcagataaatttttaattaattgctgtaataaaatcagctaaaactctatcctcctcacagcatagaacatgtacttcagtttttaggtcagctcctacaagacaattaactggcaaggaagtcagtgctcttcattacatggatacaacaatatcagagaactgttagaataacttaaacttctctaatgagcttcagagctttaatgagaagattttctattcttatgtgattaatattgctctgtgatagaaagagaactttttttttttctgaaaggaaatgtagagcaaaagagttcaagataaatgattcagaggaagttaagaagtggttaatcccttatagcccaaaggggcttaattattgataactagtaactgccaccagcataggtaggctctccaaataatcattatagcctagaataaagtaacaaaaactatttttaaactaccagcattttaacaatttattttctgaattcagtctatcaagcaattagcttagatggctataaaactgtgtgagcttgtgaacctataagtttagagacacttatgattcacgattagtcttgaaaattttttatataaaaatagtcccagtaaatcactaagtcaaaatggacgaactggttcgatgtgtgtgtatgtgtatgtctaggtacacagctgtgtgtgtgtctaggtgtgtagctgtgtgtgtgtccacacacattttgaaaatcaacaggttagcagaaacaggatgacgaaggaagaatgagaaaaagagatagttataaagagtcactgataataagcaaaacattcttaggatatgtgggtgtatgtaatcagtggcctttttgtacaaatacatcttttttgacatattagcttccatcgacatcaaccgaagctgatttcagaatgtcaaggagaactagtgagaagtaaaagtgactgtaaaagtagtggaagtagaagcagtgaagatgtagagaagtttgtgaaagaagtaaaatagacgtagatggtcatcacatttaaataaaggttagtcatgcactttgaacagacactggttaaagatatttaggaaaatagaatggattttaaattttaattggggaagctgattgtaaagaagaaggagtaaaagcaaataccagaataaaaacactagagagggaaactatttcgttattagtttcagctatctccgaatgtgtgtttaggtttcaacttcagcaggagacatcaaatttattcagaagaagagctaaattgatgatggcatatcaattcaaaagctatgctttttgaagctttttgggaaagacattctcctgtgtgctgtcttttcactcccactgggccacataagaatgggcatggaacccttccttaccagtaggtaagggggcccacacaggaagtactgggcttatggccttatgtgggaatatcttttgctgtttcagaatcaacgtgtagttctttgttctgcttaagtgggtgtttcaatagtcctcagacatgtcctctccccacaattttttttcagataccatagaggagaggtcgaggtccttccactgaagcaccaagtggggtatataaagccacactgcccatgagggctgtggtgtgagtctgctagccgtgagagacagatgcattacaaagggatggatttagtgcactctttcctctcttactgtgagtagatgctgcacttcttgaacctggtgggagtgtgtgttgtctgttctcagagaccttgaatcatacacagtttttttttcacccccgggtgacatttacctccttttaacattggctacactcgtgtaatattctgtcctgcagcacagcctgaacagccagtgaacaatgaaacaaaacttcttcccaagcctcaatgaggattgtgccatagtttgaaggtaggagaatttcaagccattaatcgtctatgaatactgaataccagtgagaatggctccttcaggtctatcagcttattgctgcttctgtgttattcttaggccaaagaacaatggtcaatccagcaatcaactcattttggctgatttgtgcctttagtccaggcttctcagttgtaccacacagccgttcttctcttcaatttcagggtcattccttcaggaattctgtcaggaataagcagcaagtttcctactgctctgggtcctccttagacccttcttatttcatgttcagaggaatgaactccacatcctgtcctaaactccaccttccttctattattttttcatatttgacattgatactcattgaaactagaatctgaattattaccaccaaagatattacctttttttttgcaccggagagcttaatctttaaaatgttctgtgagagatgggaatggtgtactatctttctagtgtggcaaaagaagagagagtaacaaaataattgtgttcatattaagattatttgaatataaatattttgccaaatttgtatttaggtgtccatgttatttttaataattggattgatataagtagatcttaagaaatatcaaaaaaaaccgttttgtcaaattggagtcaaggaggcatggaaattctcttcatttgcagtactcctaaattcctgcttattctgtggttgaatttatatcaacaaagagaattggagattaaggcaattaatcagaaattttaattaaaaaaggaatggaaaaaataagtaaaatttcctatagaattctagggtttgacatctacacagatatattcattaggtagaataataagtcttttctttgctcttatgaaaatattgttctctaattgtcaatgagcataaagtaaaatgaagatagattatacaaatatccttattgattaaaagtttaaaattgccagtgcccaagaaatatgcctgaattcaaggatatcttattgcaaagaatatcttactgcagtttgttttttttccttactgtaaataaagggtcaaccaacagctaataactcacaactgcatcttttcttgttagaacatgtcgcccagcactttttgattagatgttctcagataatagctggggcagattgaccaaaggagagcaattttttttttaatcaggataacataatgattccatcacaaactttagaaagggctatggatatcaccaattttctttttttgtctattattagtgctgtatctttagtggaattgaagacatgaccttctgcaggtttcaatacaactttataaaaggctgtaacaatgcctttaaaatcaaatctattaactgttctacagagaagccagttttctagtaacttggccataacttaaagacagggaccctgaaaacaccaagcatctgcctgcaatctgaattatctccctgactagattcagtaatcttggctcttccctgcatttaagtcattttgtgtacatcaattttctcacctatgaaagaagagagttgtccaacatgttctgatgttctttcagttctgagttctttggataatttgattggagcccaagagatgtcctaaatctgactggcatgcagtaaatgtttgttgattgtgaaaaaatatttgacttccaaatgggttgtaaaatatctcgtgtggacatactgcagcaagttaacacttataattttgggagcagaataaatgtaattaagtcacaaatgcaaatgaattctctgcttatttaattcaaagagaaatagttgtctaaaaacaatctgacccatatactggttgaaaatagtaagcattatttttttatgaagggccaagtaagtggcttacatatattaaatgtaataaatgtattcaaaaagattttcacctagttgaactgcatttatacttcatgaatatcactagtgtaatcattactttaaaaacatatagctggagaaataagcatttagacaggtaagaaaacagactgaagttttaaattttatttttaacagtcattctaaaagaattatttatcattatatatttgtatgactgttaaaagttgaaacagaaaacatggaagtatgcttttaattgatcattgttatatatagagagtgaattaaaaaatttatgcatataaagttactaataattttttgagcacatatagtttcctacatacttacctatatctattctttaatccttgtaacagccccatgaactatctactattattatcatcattccatcttgcaggtgaggaagctgaggtctagagaagttcattaatttgactaaaggcacacagtcaataaatggtagagccaaggtttgaaccctggcattctggttttatgcttttaaccagctgcttattattggtatcttattgtgatgattttggaaaacactactaatttatttgaaatgtattaatagtctttatttatttacaactttaataaagaaaaagttaacctttccatatgttcaaacaagatgcataattttctcatttatcatccgcagtttctatttctctatgctttgtgaaatgaaaagctgtaatatcaaatttttattttgttttaatcatttgtatgatttgctggcctttgtgctattaaatagaatgtatgaatgcttttttagcatttagtatttaatatgtagggaacccatttctaatgaattgccatgagttgagaaatactctgtatgtactccctgaaattataagtagccaatacatgtctctatacattcctttaggaggccttattttgttctgttagaattattttaactcaaaatatattttgtttataaatgattggatcatctgttttactcaagcaattgacattgaatcacattaatattacttcattttttattcagaggttttaaacatctttcccttttactgtttttaaacacttaacactgacaatttttatatcttgtgatataaaatataataaattataatcaagtcaagaataagaatgaaatgaaaggaagaaagttgaaaaaatcagtcaaatggtgtacaatggtttcttctttaagtaagggaaataacagtttgtttacaatggaatctatctatttcaagaccctttaagaataatttgtgttgcaaagtgagaacatttgaagttccatttgaaaaatgcagtacttctgccctggcgaaaatagataagcttccgagattcttcttatgaaaaagataagcctgctgctactactgttgctgctgcaggtgccgcaaaaaggaaaaaaaaagtcttaaaaatatgttagtagacacatattagcaggagaacacatattagcagttccctagggctgtaaaaacaagcatctgccattgattttgtactcaattgaacttcacagagtcacagtcttacattattagatgaggaattatgtgggttttagtgaattcttaatgaaacctatattttaaaaattaaaatattttgatttgatcaatcttaagttgtatcacgttagtcttatccaaactcattcaaaatgtatatgggattgtttcatgaaattttgatttttttttgaaaattttgcatttcagagttgtaaatatagcttatctgaactctagaagtcatattgttcagtgttcctgaaagtaatgaaaaaagttatctagaatatgtacatatcagttagaatgtttgtgaagcaagtgattgaaaatttattatcttccagagatggcatcaaggtgagttgattcagcagcttaaccttgtcatcgagcattccagttctttcagcctctctactctgctgtctgcaacaacagtttatacaaaggctggtttctttctcatgtgagatggctttgtgtttgcccagtgtgtgtgtgtgtgtgtgtgtgtgtgtgtgtgtgtgtgtgtgtggcagaggggcttctcatgtgtataggtgaagtaagttggagttatttaggaaagggaagagcaaaaagaaagaaagaatactgggtaatcatcaatgcccaacacaggaaagagtgtaagtaaatgaaacctctgttttttaatgataaattttgcatgtagcatgttctagtaaagtttggctccactgaacttgtattactttgtaaaatattcaaagtcatattgaaaaaaattcaaatgttctctatttattacttcccttttggaattcagttatgactccttaacatagaggctctaaattcttaattatatagctgtgaaatagacttttaagtggattgtaattttatacatttgtgagcttttattgcattcatttcttatggcagagattgctccttattctcacaatctttatttgggccatggctgttctgcatatagactacttggcagcctcccttagaactaggtgtagccatatgatcaaggtctg

SEQ ID NO: 20 - DNA, 19 bp, synthetic construct
  source 1..19: mol_type = other DNA
ccctctactggcccatgtc

SEQ ID NO: 21 - DNA, 21 bp, synthetic construct
  source 1..21: mol_type = other DNA
gtggagaaggagtggttgttg

SEQ ID NO: 22 - DNA, 20 bp, synthetic construct
  source 1..20: mol_type = other DNA
gaaccacggcactgattttc

SEQ ID NO: 23 - DNA, 20 bp, synthetic construct
  source 1..20: mol_type = other DNA
ccccaccatgttctgaatct

SEQ ID NO: 24 - DNA, 22 bp, synthetic construct
  source 1..22: mol_type = other DNA
gtctcctctgacttcaacagcg

SEQ ID NO: 25 - DNA, 22 bp, synthetic construct
  source 1..22: mol_type = other DNA
accaccctgttgctgtagccaa

SEQ ID NO: 26 - DNA, 21 bp, synthetic construct
  source 1..21: mol_type = other DNA
gaaaatgatccccaagtcctc

SEQ ID NO: 27 - DNA, 21 bp, synthetic construct
  source 1..21: mol_type = other DNA
gagtcaacacggagctgtagg

SEQ ID NO: 28 - DNA, 21 bp, synthetic construct
  source 1..21: mol_type = other DNA
cgagaagaagaacggcaagta

SEQ ID NO: 29 - DNA, 21 bp, synthetic construct
  source 1..21: mol_type = other DNA
gtggagaaggagtggttgttg